(19) United States
(12) Patent Application Publication
(10) Pub. No.: US 2024/0352097 A1
(43) Pub. Date: Oct. 24, 2024

(54) NEUTRALIZING ANTIBODY FOR FLAVIVIRUSES AND PRODUCTION METHOD THEREOF

(71) Applicant: National Sun Yat-sen University, Kaohsiung city (TW)

(72) Inventors: Day-Yu CHAO, Taipei City (TW); Yen-Hsu Chen, Kaohsiung city (TW); Wen-Hung Wang, Kaohsiung city (TW)

(73) Assignee: National Sun Yat-sen University, Kaohsiung city (TW)

(21) Appl. No.: 18/637,905

(22) Filed: Apr. 17, 2024

Related U.S. Application Data
(60) Provisional application No. 63/496,468, filed on Apr. 17, 2023.

(51) Int. Cl. C07K 16/10; A61K 39/00; A61P 31/14

(52) U.S. Cl. CPC C07K 16/1081; A61P 31/14; A61K 2039/505; C07K 2317/565; C07K 2317/76

(57) ABSTRACT

The present disclosure provides a neutralizing antibody for flaviviruses, a production method, a method of treating or preventing a flaviviruses infection in a subject, and the use thereof.

### CROSS-REFERENCE TO RELATED APPLICATION

This application claims the benefit under 35 U.S.C. § 119(e) to U.S. Provisional Application No. 63/496,468, filed on Apr. 17, 2023, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 16, 2024, is named “24P0224-Seqlist-F” and is 106,314 bytes in size. The sequence listing contained in this .XML file is part of the specification and is hereby incorporated by reference herein in its entirety.

### FIELD OF INVENTION

The present disclosure relates a neutralizing antibody for flaviviruses, a production method, a method of treating or preventing a flaviviruses infection in a subject, and the use thereof.

### BACKGROUND OF THE INVENTION

Flaviviruses, the largest member of the family Flaviviridae, present a global threat to public health. Many of the mosquito-borne flaviviruses are established human pathogens, including the Japanese encephalitis virus (JEV), Yellow fever virus (YFV), West Nile virus (WNV), the four serotypes of dengue virus (DENV), and the re-emerged Zika virus (ZIKV). Co-circulation of multiple flaviviruses in the same geographic locations, combined with human global mobility and travel vaccine coverage for YFV and JEV, have increased the likelihood of exposure to multiple flaviviruses within a lifetime. However, how pre-existing immunity will influence the antibody response upon subsequent infections or vaccination with heterologous flaviviruses remains poorly understood.

Flaviviruses are enveloped viruses containing a single-strand, positive-sense RNA with an 11-Kb genome and encapsidated by three structural proteins, namely the capsid (C), pre-membrane/membrane (prM/M), and envelope (E) proteins. The immune response to flaviviral infection mainly targets the E proteins and is known to be dominated by transient and highly cross-reactive (CR) antibodies during the acute and early convalescent phase. In the late convalescent phase, the immune response generates type-specific neutralizing antibodies but lacks durable and high cross-neutralizing antibodies against viruses from different serotypes or serocomplexes. The envelope dimer epitope (EDE) human monoclonal antibodies (huMAbs), which broadly neutralize the four dengue virus serotypes by recognizing the quaternary epitopes on the virion surface, have been mostly isolated from individuals exposed to secondary dengue infections. Recurring huMAbs CR to DENV and ZIKV have also been reported from the regions where both viruses co-circulated. However, the antibody profiles of individuals residing in areas where JEV and DENV co-circulated have not been investigated in detail.

### SUMMARY OF THE INVENTION

In the present disclosure, the inventors used a unique Taiwan cohort to gain insight into how DENV infection in individuals with pre-existing JEV immunity shapes neutralizing antibody responses against multiple flaviviruses through interdisciplinary and complementary approaches, including epidemiology, immunology, structural biology, and animal studies. The results will guide future vaccination strategies, leading to the generation of broadly neutralizing antibodies against different flavivirus serocomplexes.

To achieve the above propose, one aspect of the present disclosure provided herein is a neutralizing antibody for flaviviruses, including:
(1) a heavy chain complementary determining region 1 (HC CDR1) set forth as SEQ ID NO: 1, a heavy chain complementary determining region 2 (HC CDR2) set forth as SEQ ID NO: 2, a heavy chain complementary determining region 3 (HC CDR3) set forth as SEQ ID NO: 3, a light chain complementary determining region 1 (LC CDR1) set forth as SEQ ID NO: 4, a light chain complementary determining region 2 (LC CDR2) set forth as SEQ ID NO: 5, and a light chain complementary determining region 3 (LC CDR3) set forth as SEQ ID NO: 6;
(2) a HC CDR1 set forth as SEQ ID NO: 7, a HC CDR2 set forth as SEQ ID NO: 8, a HC CDR3 set forth as SEQ ID NO: 9, a LC CDR1 set forth as SEQ ID NO: 10, a LC CDR2 set forth as SEQ ID NO: 11, and a LC CDR3 set forth as SEQ ID NO: 12;
(3) a HC CDR1 set forth as SEQ ID NO: 7, a HC CDR2 set forth as SEQ ID NO: 8, a HC CDR3 set forth as SEQ ID NO: 9, a LC CDR1 set forth as SEQ ID NO: 13, a LC CDR2 set forth as SEQ ID NO: 14, and a LC CDR3 set forth as SEQ ID NO: 15; or
(4) a HC CDR1 set forth as SEQ ID NO: 7, a HC CDR2 set forth as SEQ ID NO: 8, a HC CDR3 set forth as SEQ ID NO: 9, a LC CDR1 set forth as SEQ ID NO: 16, a LC CDR2 set forth as SEQ ID NO: 17, and a LC CDR3 set forth as SEQ ID NO: 18.

Preferably, the neutralizing antibody includes a heavy chain variable domain (V_{H}) that is at least 85% identical to the amino acid sequence of SEQ ID NO: 19, and/or a light chain variable domain (V_{L}) that is at least 85% identical to the amino acid sequence of SEQ ID NO: 20.

Preferably, the neutralizing antibody includes a V_{H} that is at least 85% identical to the amino acid sequence of SEQ ID NO: 21, and/or a V_{L} that is at least 85% identical to the amino acid sequence of SEQ ID NO: 22.

Preferably, the flaviviruses includes Dengue fever virus serotype 1 (DENV-1), Dengue fever virus serotype 2 (DENV-2), Dengue fever virus serotype 3 (DENV-3), Dengue fever virus serotype 4 (DENV4), Japanese encephalitis virus (JEV), and/or Zika virus (ZIKV).

Preferably, the neutralizing antibody is capable of neutralizing more than one subtypes of the flaviviruses.

Preferably,
(a) the neutralizing antibody binds within residues 72-89, 98-111, 186-190, 224-237, 243-249, 286-299, and/or 357-364 of the DENV-1 including the amino acid sequence of SEQ ID NO: 23,
(b) the neutralizing antibody binds within residues 72-89, 98-111, 186-190, 224-237, 243-249, 286-299, and/or 357-364 of the DENV-2 including the amino acid sequence of SEQ ID NO: 24,
(c) the neutralizing antibody binds within residues 72-89, 98-111, 184-188, 222-235, 241-247, 284-297, and/or 355-362 of the DENV-3 including the amino acid sequence of SEQ ID NO: 25, or
(d) the neutralizing antibody binds within residues 72-89, 98-111, 186-190, 225-238, 252-258, 287-300, and/or 358-365 of the DENV-4 including the amino acid sequence of SEQ ID NO: 26.

Preferably, the neutralizing antibody binds within residues 72-89, 98-111, 191-195, 229-239, 245-251, 288-301, and/or 360-369 of the JEV including the amino acid sequence of SEQ ID NO: 27.

Preferably, the neutralizing antibody binds within residues 72-89, 98-111, 191-195, 229-242, 248-254, 292-305, and/or 364-373 of the ZIKV including the amino acid sequence of SEQ ID NO: 28.

Preferably, the neutralizing antibody is a monoclonal antibody.

Preferably, the neutralizing antibody is a full-length antibody or an antigen binding fragment thereof.

Preferably, the full-length antibody includes an IgG molecule.

Preferably, the antigen binding fragment of the neutralizing antibody is selected from the group consisting of F(ab′)2, Fab′, Fab, Fv, single domain antibody (VHH), and scFv.

In another aspect of the present disclosure, provided herein is a method for producing a neutralizing antibody, including:
(a) providing a mammal animal;
(b) delivering an antigen to the mammal animal, wherein the antigen comprises Japanese encephalitis virus (JEV) antigen, Dengue fever virus (DENV) antigen, and/or a combination thereof;
(c) selecting a B cell that expresses the neutralizing antibody from the mammal animal, wherein the neutralizing antibody is capable of neutralizing Zika virus (ZIKV); and
(d) obtaining the neutralizing antibody from the B cell.

Preferably, the mammal animal includes mouse, rat, rabbit, monkey, chimpanzee, and/or human.

Preferably, in the step (b), the JEV antigen is delivered prior to the DENV antigen to the mammal animal.

Preferably, the antigen is delivered at levels sufficient to induce an immune response of the mammal animal.

Preferably, the JEV antigen includes JEV and/or JEV vaccine.

Preferably, the JEV vaccine includes inactivated Vero cell culture derived JE vaccine (JE-VC), inactivated mouse brain derived JE vaccine (JE-MB), primary hamster kidney cell derived, attenuated JE vaccine, and/or live attenuated chimeric JE vaccine.

Preferably, the DENV antigen includes DENV vaccine, DENV serotype 1 (DENV-1), DENV serotype 2 (DENV-2), DENV serotype 3 (DENV-3), and/or DENV serotype 4 (DENV-4).

Preferably, the DENV vaccine includes live attenuated dengue vaccine.

Preferably, in the step (d), the neutralizing antibody is obtained by expressing an isolated DNA encoding the neutralizing antibody derived from the B cell.

Preferably, the isolated DNA encodes a full-length antibody of the neutralizing antibody or an antigen binding fragment thereof.

Preferably, the full-length antibody includes an IgG molecule.

Preferably, the antigen binding fragment of the neutralizing antibody is selected from the group consisting of F(ab′)2, Fab′, Fab, Fv, single domain antibody (VHH), and scFv.

In another aspect of the present disclosure, provided herein is a pharmaceutical composition including the any one of aforementioned neutralizing antibody, optionally together with a pharmaceutically acceptable carrier, diluent or excipient.

In another aspect of the present disclosure, provided herein is a method of treating or preventing a flaviviruses infection in a subject, including administering to the subject an effective amount of the aforementioned pharmaceutical composition under conditions effective to neutralize the flaviviruses.

Preferably, the flaviviruses includes Dengue fever virus (DENV-1), Dengue fever virus serotype 2 (DENV-2), Dengue fever virus serotype 3 (DENV-3), Dengue fever virus serotype 4 (DENV-4), Japanese encephalitis virus (JEV), and/or Zika virus (ZIKV).

Preferably, the pharmaceutical composition is capable of treating or preventing more than one subtypes of the flaviviruses infection in the subject.

In another aspect of the present disclosure, provided herein is a use of the any one of aforementioned neutralizing antibody for the manufacture of a medicament for treating or preventing a flaviviruses infection in a subject.

### BRIEF DESCRIPTION OF THE DRAWINGS

FIG. 1. Dengue-immune donors with prior JEV exposure have elevated ZIKV-neutralizing titers. a Neutralization antibody profile against the four serotypes of DENV, JEV, and ZIKV of healthy (n=80) and dengue-infected febrile (DF, n=60) individuals. Two-fold serially diluted plasma samples from healthy and DF individuals were evaluated for neutralization against the prototype strains of DENV-1 to -4 serotypes, JEV, and ZIKV using FRμNT90. Empty and shaded circles represent the FRμNT90 values of the healthy and DF individuals, respectively. b Comparison of the neutralization antibody profile of DF individuals against DENV-1 to -4 serotypes, JEV, and ZIKV prototype virions based on age classifications: 20-30 years old (n=30) and 50-70 years old (n=30). Empty and shaded circles represent the FRμNT90 values of the 20-30 and 50-70 age groups, respectively. c Assessment of the breadth of ZIKV neutralization among the four groups of donors dichotomized according to the presence (pos) or absence (neg) of protective or high neutralizing FRμNT90 titers against JEV and DENV, respectively. JEV-immune individuals with high (JEV^{pos}DENV^{high}; n=39) or low DENV immunity (JEV^{pos}DENV^{low}; n=24) are represented by filled or empty squares, respectively. JEV-negative donors with high (JEV^{neg}DENV^{high}; n=12) or low DENV (JEV^{neg}DENV^{low}; n=65) immunity are shown in filled or empty squares, respectively. d Effect of JEV and DENV immunity in DF individuals to ZIKV-neutralizing antibody titers. Fisher's exact test was performed in n=60 with a p-value of 0.000013. All data are representative of three independent experiments. In (a-c), the black solid horizontal bars represent the geometric mean titers (GMT) of samples with SD analyzed within each group. FRμNT90 titer <10 was represented with 5 for graphic display and statistical analysis. Significant differences were tested using one-way ANOVA followed by Tukey's multiple comparisons post-test, with levels of significance defined by ***P<0.0001; **P<0.001. Each point shows the mean of data from two independent experiments. DENV dengue virus, JEV Japanese encephalitis virus, ZIKV Zika virus, FRμNT focus-reduction microneutralization test, VLP virus-like particle, ANOVA analysis of variance, SD standard deviation.

FIG. 2. Functional characterization of anti-ZIKV human monoclonal antibodies (huMAbs) from donor KH1891. a Neutralizing antibody titers of donor KH1891 against the prototype strains of the DENV serotypes 1-4, JEV, and ZIKV at less than 1 month and 18 months post-infection plasma collection, representing the early and late convalescent stages, respectively. Significant differences were tested using two-way ANOVA followed by Bonferroni post-tests from three independent experiments. ****P<0.001. b-e Characterization of purified huMAbs, expressed as IgG1, isolated from donor KH1891. b The binding reactivity against virus-like particles (VLPs) and d in vitro neutralization activities of 10 purified huMAbs against DENV-1 to -4, JEV, and ZIKV were measured by ELISA and FRμNT, respectively. Each VLP was properly titrated to obtain equimolar concentrations per well before use in antigen-capture ELISAs. Each point shows the mean±SD of data from two and three independent experiments for binding ELISA and microneutralization assays, respectively. A nonlinear curve was generated for all antibody dilution series for all of the assays. The corresponding antibody identities are shown in the box. c, e Heatmap of VLP-binding profile (c) and of neutralization profile (e) of the 11 huMAbs against recombinantly produced DENV-1 to -4, JEV, and ZIKV VLPs and prototype strains of DENVs, JEV, and ZIKV virus particles, respectively. The heatmaps were generated based on the minimal binding concentration (or endpoint titer values) against each VLP and the IC50 values for each virion; exact numerical values expressed in g/mL are shown in each box. Each huMAb was tested in duplicates for each of the binding ELISA assays, while FRμNT data were representative of three independent experiments. A purified human IgG1 was used as an isotype control in all assays. Data are representative of three independent experiments. f, g FRμNT50 (g/mL) values of K8b (f) and K5 (g) for neutralization of the prototype strains of the four DENV serotypes, JEV, and ZIKV. Each point shows the mean±SD of data from three independent experiments. The geometric mean inhibitory concentration (g/mL) at 50% (IC50) is indicated in parenthesis. DENV dengue virus, JEV Japanese encephalitis virus, ZIKV Zika virus, ELISA enzyme-linked immunosorbent assay, FRμNT focus-reduction microneutralization test, VLP virus-like particle, ANOVA analysis of variance, SD standard deviation.

FIG. 3. Defining the human Ig genes and properties of broadly neutralizing ZIKV-CR huMAbs. a, c Pie plots showing immunoglobulin heavy (a) and kappa light chain (c) gene family utilization and distribution of ZIKV-CR neutralizing huMAbs isolated from KH1891. The proportion of IGHV and IGκV gene usage is reported beside the different gray scale. b, d Gene family assignments for the 10 purified IgG1 B cell clones from KH1891. IMGT was used to assign the germline reference sequence for IGHV (b) and IGκV (d) and the relative similarity with the germline clonotype expressed in %. e Relative percentage of somatic hypermutation (% SHM) in the paired IGHV and IGκV genes of ZIKV-CR huMAbs. f Comparison of the V-J genes and alleles of heavy and light chains of ZIKV-CR huMAbs. Values highlighted in red show increased SHM (>20%) relative to the germline gene family. ZIKV Zika virus.

FIG. 4. Mapping of target flaviviruses epitopes of huMAb K8b. a Structure-based strategy and workflow for the epitope mapping of K8b using mature DENV-2 virus-like particles (mD2VLP). AlphaFold was used to predict the three-dimensional (3D) structure of huMAb K8b expressed as IgG1. K8b-IgG1 was utilized to form an immune complex with mD2VLPs and 3D reconstructed by cryo-EM. The derived structure was further improved by MODELLER to predict the key epitopes and build epitope models. The antibody and apparatus icons were sourced icons and created with biorender.com. b AlphaFold model structure for K8b-IgG1 with coloration based on model confidence. The complementarity determining regions (CDRs) were indicated. c The cryo-EM structure of K8b-IgG1 and mD2VLP complex revealed the arrangement of E glycoprotein at T=1 icosahedral symmetry. In the configuration, 60 F(ab) molecules were bound to 60 envelope proteins on the VLP surface (shown in cyan), while the Fc region was not resolved in the structure. d Fitting the predicted structure of K8b-IgG1 into cryo-EM density maps is presented, with contour levels of 3.7 σ (left) and 1.2 σ (middle). Two perspectives are shown: side view (d, left and middle) and top view (d, right). The separation of variable domains from the constant domain was clearly defined in the density map at higher contour levels, as indicated by the arrows. Heavy and light chain pairs of K8b-IgG1 bound to E proteins at the inter-dimer surfaces. The triangle represents an asymmetric unit. e A flexible refinement of immune complex structure into cryo-EM map showing ten conformers of K8b-IgG1 predicted by MODELLER. The cross-correlation function (CCF) is 0.72. For (d) and (e), a scale bar of 5 nm is shown. f Interacting or contact residues between the E proteins on an mD2VLP and the heavy or light chain of K8b-IgG1, including the equivalent residues (and their AA positions) in other flaviviruses, predicted by MODELLER. g A representative amino acid sequence alignment of the E proteins among prototype flaviviruses including selected DENV serotypes, JEV, and ZIKV using Clustal Omega and visualized by JalView 2.11.1.4. Potential binding sites of K8b are highlighted in boxes, arranged sequentially from the N-terminus to the C-terminus: be loop, ED I/II linker, hi loop, ED I/III linker, and DE loop. Residues in gray are surface-exposed (solvent-accessible surface area, QSASA>0.30). Sequence identities were normalized by aligned length, while the positions of the amino acids in E were labeled according to ZIKV PRVABC59. The shaded residues show 100% conservancy among the selected flaviviruses genera. Above the sequences, the arrows indicate β-strands, rectangles represent the helices, and lines show the spanning loops and strands in the E structure. The E domains I, II, and III are represented by arrows and lines in red, yellow, and blue, respectively. h, i Binding ELISA reactivity profile of purified K8b-IgG1 (h) and K5-IgG1 (i) at 10 and 1.0 μg/mL antibody concentrations against seven single-site mutants relative to the wild-type ZIKV VLP in ELISA. The consensus amino acid residues in the be loop (ZIKV R73, T76, and D87), ED I/II linker (ZIKV R193), hi loop (ZIKV T231), ED/III linker (ZIKV G302), and ED III DE loop (ZIKV T366) were mutated to alanine. For (h, i), the statistical differences were determined by two-way ANOVA with Tukey's multiple comparisons test relative to the binding reactivity to the ZIKV WT VLP, and are defined as significant at: *P<0.05, **P<0.001, and ***P<0.0001. The binding reactivity ELISA data shown in (h, i) are representative of four independent experiments, expressed as geometric means±SD. DENV dengue virus, JEV Japanese encephalitis virus, ZIKV Zika virus, ELISA enzyme-linked immunosorbent assay, FRμNT focus-reduction microneutralization test, ANOVA analysis of variance, SD standard deviation.

FIG. 5. Generation and functional characterization of purified K8b-IgG1, K8b-F(ab′)2, and K8b-F(ab). a Schematic representation of the designed gene constructs coding for full-length human IgG1, F(ab′)₂, and F(ab) encoding the Ig variable heavy chain genes of K8b (K8b-V_{H}), and hence termed as pVCHIg-hG1-K8b, pVCHIg-K8b-F(ab′)₂-His and pVCHIg-K8b-F(ab)-His, respectively. The schematic diagram of the expression vector, pVCLIg-hx, encoding the variable Ig kappa light chain domain of K8b (K8b-Vx) is also shown in the upper right panel. Each of the molecules was expressed via co-transfection using the kappa light chain expressing plasmid, pVCLIg-hx-K8b. Inset shows the detailed schematic map of the human Igγ constant domains 1 (C_{H}1) and 2 (CH2), including the IgG1 hinge regions and the actual location where cysteine (Cys) was introduced for increased thermal stability. C_{H}1-3, human Igγ constant domains 1-3; Cκ, human Igκ constant domain; 8×-His, poly-Histidine tag; SP, signal peptide; H, hinge; TGA, stop codon are highlighted. b SDS-PAGE of purified human monoclonal antibody, K8b, expressed as full-length IgG1, F(ab), and F(ab′)₂ molecules visualized under nonreducing conditions. Arrow marks show the dimeric species of K8b-IgG1 at 150 kDa, monovalent K8b-F(ab) at 48 kDa, and the bivalent K8b-F(ab′)₂ at 110 kDa. The molecular mass of the protein marker is indicated on the left side, lane 1, for each gel panel. c Electrophoretic mobility of purified K8b-F(ab′)₂ after size-exclusion chromatography (SEC) showing the separation of K8b-F(ab′)₂ and K8b-F(ab) molecules from the pooled pre-SEC K8b-F(ab′)₂ fractions in lane 1. Lanes 2 and 3 show the non-reduced form of K8b-F(ab′)₂ post-SEC and lane 4 shows the K8b-F(ab) molecules post-SEC purity separation. The molecular mass of the protein marker is indicated on the right side, lane 5. d Cartoon representation of K8b IgG1, F(ab), and F(ab′)₂ molecules. e Neutralization profile of K8b expressed as full-length IgG1, F(ab), and F(ab′)₂. Prototype virus strains of DENV-1 to −4, JEV, and ZIKV were used for the FRμNT. f Binding profile of K8b expressed as full-length IgG1, F(ab), and F(ab′)₂. Each VLP was properly titrated to obtain equimolar concentrations per well before use in antigen-capture ELISAs. In (e) and (f), each point shows the mean±SD of data from two independent experiments. A nonlinear curve was generated for all antibody dilution series for all of the assays. DENV dengue virus, JEV Japanese encephalitis virus, ZIKV Zika virus, ELISA enzyme-linked immunosorbent assay, FRμNT focus-reduction microneutralization test, SD standard deviation, SASA solvent-accessible surface area.

FIG. 6. Neutralization antibody profile of murine sera and murine monoclonal antibodies (mAbs) generated from various DENV-2 and JEV VLP prime-boost immunizations. a BALB/c mice (n=5) were primed and boosted at 0 and 28 days, respectively, with various combinations of mature DENV-2 VLP (mD2VLP) and JEV VLP antigens. Mice were bled at 84 days post-vaccination, and individual serum specimens were evaluated for 50% focus-reduction microneutralization (FRμNT50) activity against DENV, JEV, and ZIKV. The bar plots show GMT±SD for each group. The p values (*P<0.05, **P<0.01, ***P<0.001, ****P<0.0001) were determined on log-transformed data using one-way ANOVA with Tukey's multiple comparisons post-test. b Schedule of boost and myeloma fusion experiments performed in representative mice from the homologous JEV VLP prime-boost immunization. c, d Representative mice (JEV FRμNT50 >120) from JEV prime-boost immunization were further boosted for three consecutive times (112-114 days post-vaccination) with JEV VLP (c) or mD2VLP (d) and the splenocytes were harvested two days after the last boost and then individually fused with myeloma cells for hybridoma generation. Culture supernatants from selected polyclonal hybridomas were tested for positive VLP ELISA reactivity (P/N ratio≥2) and further subcloned by limiting dilution to isolate monoclonal cells. c 71% of the 52 JEV-specific monoclones isolated from 3D2 polyclone secreted JEV-neutralizing mAbs with varying potencies at 50% inhibitory concentration (IC50, μg/mL) of unpurified supernatant. c Right: Pie plot showing murine monoclones selected from polyclonal hybridomas and the distribution of 52 type-specific 3D2 mAbs with varying breadth of neutralization. d A total of 36 monoclones were isolated from 5D5 polyclone. Percentages represent the proportions of secreted mAbs according to their breadth of cross-neutralization within the different serocomplexes. e Pie plot showing murine monoclones selected from polyclonal hybridomas and the distribution of 36 cross-neutralizing 5D5 monoclonal antibodies. The relative proportion of monoclones with varying breadth of neutralization is reported beside the different color schemes. f Heatmap of DENV, JEV, and ZIKV IC50 (μg/mL, unpurified supernatant) neutralization titers of the 36 cross-reactive mAbs. Data are representative of two independent experiments. g Distribution of immunoglobulin (IgG) isotypes identified from hybridoma screening. DENV dengue virus, JEV Japanese encephalitis virus, ZIKV Zika virus, VLP virus-like particle, ELISA enzyme-linked immunosorbent assay, GMT geometric mean titer, ANOVA analysis of variance, SD standard deviation.

FIG. 7. The pipeline for the discovery andgeneration of human monoclonal antibodies using the single B-cell technology. From immune donors, PBMCs were prepared for the isolation of monocytes by flow cytometry using a negative selection strategy. The bivariate plot showing the fluorescently labeled, single-sorted human B cells was generated by a licensed software (BD FACSChorus™, v.1.1.18.0). Human B cells were co-cultivated with CD40Lexpressing thymoma cells and growth factors for two weeks and screened for antigen specificity using ELISA. Following RT-PCRs, productive human immunoglobulin (Ig) V_{H} and V_{K} segments were cloned into in-house generated expression vectors and co-transfected into HEK293T cells for recombinant antibody expression. The plasmid maps were generated using an open-source plasmid visualization software, Plasm (v.2.0). Purified antibodies were tested for VLP binding and live-virus neutralization, followed by epitope mapping and structural data analyses. The representative heatmap of ELISA binding reactivity and % relative infection curves were generated using GraphPad Prism (v.9.5.1, GraphPad Software, LLC), while the binding footprint of K8b with its heavy and light chains on a three-fold ZIKV E-protein axis was generated using the PyMOL (v.2.5.4)-Easy MODELLER (v.4.0) software package. In all subpanels of b, the remaining icons were original creations or source icons created with Biorender.com.

### DETAILED DESCRIPTION

The foregoing and other aspects of the present disclosure will now be described in more detail with respect to other embodiments described herein. It should be appreciated that the invention can be embodied in different forms and should not be construed as limited to the embodiments set forth herein. Rather, these embodiments are provided so that this disclosure will be thorough and complete and fully convey the invention's scope to those skilled in the art.

The terminology used in the description of the invention herein is for the purpose of describing particular embodiments only and is not intended to be limiting to the invention. As used in the description of the invention and the appended claims, the singular forms “a”, “an” and “the” are intended to include the plural forms as well, unless the context clearly indicates otherwise.

As used herein, the terms “comprises,” “comprising,” “includes,” “including,” “has,” “having,” “contains”, “containing,” or any other variation thereof, are intended to cover a non-exclusive inclusion, subject to any limitation explicitly indicated. For example, a composition, mixture, process or method that includes a list of elements is not necessarily limited to only those elements but may include other elements not expressly listed or inherent to such composition, mixture, process, or method.

As used herein, the term “about” indicates that a value includes, for example, the inherent variation of error for a measuring device, the method employed to determine the value, or the variation among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive. However, the disclosure supports a definition that refers to only alternatives and “and/or.”

Unless otherwise defined, all technical and scientific terms used herein have the same meaning as commonly understood by one of ordinary skill in the art to which this invention belongs. All publications, patent disclosure, patents, and other references cited herein are incorporated by reference in their entirety for the teachings relevant to the sentence and/or paragraph in which the reference is presented.

The propose of the present disclosure is to provide a neutralizing antibody for flaviviruses, including one of the following combinations of complementary determining region sequences are shown in table 1:

TABLE 1
neutralizing antibody complementary
determining region sequences
Heavy chain complementary determining region

| CDRs | | | |
|---|---|---|---|
| combination set | HC CDR1 | HC CDR2 | HC CDR3 |
| 1 (K8b) | GYTFTSNW | IDPSDSYT | CARGSNPYYYAMD |
| | (SEQ ID | (SEQ ID | YW |
| | NO: 1) | NO: 2) | (SEQ ID NO: 3) |
| 2 (K5) | GDTFSNSD | IIPAYDTT | CATDQRDYFGLGN |
| 3 (K12) | (SEQ ID | (SEQ ID | HFNAQHFW |
| 4 (K22 or K23) | NO: 7) | NO: 8) | (SEQ ID NO: 9) |

Light chain complementary determining region

| CDRs | | | |
|---|---|---|---|
| combination set | LC CDR1 | LC CDR2 | LC CDR3 |
| 1 (K8b) | QSVSSNY | GVS | CQHYGGSLSWTF |
| | (SEQ ID | (SEQ ID | (SEQ ID NO: 6) |
| | NO: 4) | NO: 5) | |
| 2 (K5) | QSISNY | TAS | CQQSYSGGYTF |
| | (SEQ ID | (SEQ ID | (SEQ ID NO: 12) |
| | NO: 10) | NO: 11) | |
| 3 (K12) | QNINSY | DAS | CQQRRNWPWTF |
| | (SEQ ID | (SEQ ID | (SEQ ID NO: 15) |
| | NO: 13) | NO: 14) | |
| 4 (K22 or K23) | QDVSNF | YTS | COQHSTFPLTF |
| | (SEQ ID | (SEQ ID | (SEQ ID NO: 18) |
| | NO: 16) | NO: 17) | |

The term “neutralizing antibody” refers to an antibody that attaches directly to a pathogen, including virus, thereby blocking the virus's capability to invade and infect target cells. Whether an antibody is a neutralizing antibody can be determined by in vitro assays described in the Examples section herein below.

The term “potent neutralizing antibody” refers to an antibody which, when used at a low concentration, reduces flaviviruses infection by at least 50%, 60%, 70%, 80%, 90%, 95%, 99% or greater. Concentrations below 50 μg/ml, between 1 and 50 μg/ml, or even below 1 μg/mi, are considered “low concentrations”. In some embodiments, low concentrations are concentrations in the picomolar range, such as 10-900 ng/ml, and include any concentration in that range, such as 800, 700, 600, 500, 400, 300, 200, 100, 75, 50, 25, 10 ng/ml, or even less than 10 ng/ml.

The term “broad neutralizing antibody” refers to an antibody which inhibits flaviviruses infection, as defined by a 50% inhibition of infection in vitro, in more than 50%, 60%, 70%, 80%, 90%, 95%, 99% or greater, of a large panel of (greater than 100) flaviviruses envelope pseudotyped viruses and viral isolates.

The neutralizing antibody molecule includes a heavy chain variable region (V_{H}) and a light chain variable region (V_{L}), which are usually involved in antigen binding. The V_{H} and V_{L}regions further includes hypervariable region, also known as “complementarity determining regions” (“CDR”), and the regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology, including the Kabat definition, the IMGT definition, and/or others methodology which are well known in the art. Preferably, the IMGT definition methodology is used in the present disclosure.

The term “flaviviruses” refers to all viruses within the Flaviviridae family, including the hepatitis C virus, the dengue viruses (all four serotypes), the West Nile virus, the yellow fever virus, the Japanese encephalitis virus, the tick-borne encephalitis virus, the Murray Valley encephalitis virus, the Saint Louis encephalitis virus, the Powassan virus, the classical swine fever virus, the bovine viral diarrhea virus, the border disease virus, and the hepatitis G virus.

In an embodiment, the neutralizing antibody includes a V_{H} that is at least 85%, preferably at least 90%, 95%, 99%, identical to the amino acid sequence of SEQ ID NO: 19, and/or V_{L} that is at least 85%, preferably at least 90%, 95%, 99%, identical to the amino acid sequence of SEQ ID NO: 20. The V_{H} and V_{L} sequences are

V_{H}:
(SEQ ID NO: 19)
EVQLLESGAEVKKPGSSVKVSCQASGDTFSNSDINWVRQAPGQGLEWMGG
IIPAYDTTNYARTFQGRVAITADKSTSTVYMELSRLRSEDTAVYYCATDQ
RDYFGLGNHFNAQHFWGQGTLVTVSS
V_{L}:
(SEQ ID NO: 20)
AIRMTQTPLSLSASVGDRVTITCRASQSISNYLNWYQQRPGKAPNLLIYT
ASNLHSGVPSRFSGSGSGTDFTLTISSLQPEDFATYYCQQSYSGGYTFGQ
GPSWEIK

In an embodiment, the neutralizing antibody includes V_{H} that is at least 85%, preferably at least 90%, 95%, 99%, identical to the amino acid sequence of SEQ ID NO: 21, and/or a V_{L} that is at least 85%, preferably at least 90%, 95%, 99%, identical to the amino acid sequence of SEQ ID NO: 22. The V_{H} and V_{L} sequences are:

V_{H}:
(SEQ ID NO: 21)
QVQLVQPGAEVVKPGASVKLSCKTSGYTFTSNWMHWVKQAPGQGLEWIGE
IDPSDSYTNYNQNFQGKAKLTVDKSTSTAYMEVSSLRSDDTAVYYCARGS
NPYYYAMDYWGQGTSVTVSS
V_{L}:
(SEQ ID NO: 22)
EIVMMQSPLTLSLSPGERATLSCRASQSVSSNYLAWYQQRPGQAPRLLIY
GVSSRATGVPVRFSGSGSGTDFTLTISRLEPEDFAVYYCQHYGGSLSWTF
GQGTKVEIK

In an embodiment, the flaviviruses include Dengue fever virus serotype 1 (DENV-1), Dengue fever virus serotype 2 (DENV-2), Dengue fever virus serotype 3 (DENV-3), Dengue fever virus serotype 4 (DENV4), Japanese encephalitis virus (JEV), and/or Zika virus (ZIKV).

In an embodiment, the neutralizing antibody is capable of neutralizing more than one subtypes (e.g. more than two, three, four, five, six subtypes) of the flaviviruses. Preferably, the neutralizing antibody is capable of neutralizing DENV-1 (and/or DENV-2, 3, 4) and JEV. Preferably, the neutralizing antibody is capable of neutralizing DENV-1 (and/or DENV-2, 3, 4) and ZIKV. Preferably, the neutralizing antibody is capable of neutralizing JEV and ZIKV. Preferably, the neutralizing antibody is capable of neutralizing DENV-1 (and/or DENV-2, 3, 4), JEV and ZIKV.

In an embodiment,
(a) the neutralizing antibody binds within residues 72-89, 98-111, 186-190, 224-237, 243-249, 286-299, and/or 357-364 of the DENV-1 including at least 80%, preferably at least 90%, 95%, 99%, identical to the amino acid sequence of SEQ ID NO: 23,
(b) the neutralizing antibody binds within residues 72-89, 98-111, 186-190, 224-237, 243-249, 286-299, and/or 357-364 of the DENV-2 including at least 80%, preferably at least 90%, 95%, 99%, identical to the amino acid sequence of SEQ ID NO: 24,
(c) the neutralizing antibody binds within residues 72-89, 98-111, 184-188, 222-235, 241-247, 284-297, and/or 355-362 of the DENV-3 including at least 80%, preferably at least 90%, 95%, 99%, identical to the amino acid sequence of SEQ ID NO: 25, or
(d) the neutralizing antibody binds within residues 72-89, 98-111, 186-190, 225-238, 252-258, 287-300, and/or 358-365 of the DENV-4 including at least 80%, preferably at least 90%, 95%, 99%, identical to the amino acid sequence of SEQ ID NO: 26.

In an embodiment, the neutralizing antibody binds within residues 72-89, 98-111, 191-195, 229-239, 245-251, 288-301, and/or 360-369 of the JEV including at least 80%, preferably at least 90%, 95%, 99%, identical to the amino acid sequence of SEQ ID NO: 27.

In an embodiment, the neutralizing antibody binds within residues 72-89, 98-111, 191-195, 229-242, 248-254, 292-305, and/or 364-373 of the ZIKV including at least 80%, preferably at least 90%, 95%, 99%, identical to the amino acid sequence of SEQ ID NO: 28.

Exemplary amino acid sequences of the aforementioned flaviviruses are:

DENV-1:
(SEQ ID NO: 23)
MRCVGIGSRDFVEGLSGATWVDVVLEHGSCVTTMAKDKPTLDIELLKTEVINPAVLRKLCIE
AKISNTTTDSRCPTQGEATLVEEQDANFVCRRTFVDRGWGNGCGLFGKGSLITCAKFKCVTK
LEGKIVQYENLKYSVIVTVHTGDQHQVGNETTEHGTIATITPQAPTSEIQLTDYGALTLDCS
PRTGLDFNEMVLLTMKEKSWLVHKQWFLDLPLPWTSGASTPQETWNREDLLVTFKTAHAKKQ
EVVVLGSQEGAMHTALTGATEIQTSGTTKIFAGHLKCRLKMNKLTLKGMSYVMCTGSFKLEK
EVAETQHGTVLVQVKYEGTDAPCKIPFSTQDEKGVTQNGRLITANPIVTDKEKPVNIEAEPP
FGESYIVVGAGEKALKLSWFKKGSSIGK
DENV-2:
(SEQ ID NO: 24)
MRCIGMSNRDFVEGVSGGSWVDIVLEHGSCVTTMAKNKPTLDFELIKTEAKQPATLRKYCIE
AKLINTTTESRCPTQGEPSLNEEQDKRFVCKHSMVDRGWGNGCGLFGKGGIVTCAMERCKKN
MEGKVVQPENLEYTIVITPHSGEEHAVGNDTGKHGKEIKITPQSSITEAELTGYGTVTMECS
PRTGLDFNEMVLLQMENKAWLVHRQWFLDLPLPWLPGADTQGSNWIQKETLVTFKNPHAKKQ
DVVVLGSQEGAMHTALTGATEIQMSSGNLLFTGHLKCRLRMDKLQLKGMSYSMCTGKFKVVK
EIAETQHGTIVIRVQYEGDGSPCKIPFEIMDLEKRHVLGRLITVNPIVTEKDSPVNIEAEPP
FGDSYIIIGVEPGQLKLNWFKKGSSIGW
DENV-3:
(SEQ ID NO: 25)
MRCVGVGNRDFVEGLSGATWVDVVLEHGGCVTTMAKNKPTLDIELQKTEATQLATLRKLCIE
GKITNITTDSRCPTQGEAILPEEQDQNYVCKHTYVDRGWGNGCGLFGKGSLVTCAKFQCLES
IEGKVVQHENLKYTVIITVHTGDQHQVGNDTQGVTVEITPQASTVEAILPEYGTLGLECSPR
TGLDFNEMILLTMKNKAWMVHRQWFFDLPLPWTSGATTETPTWNRKELLVTFKNAHAKKQEV
VVLGSQEGAMHTALTGATEIQNSGGTSIFAGHLKCRLKMDKLELKGMSYAMCLNTFVLKKEV
SETQHGTILIKVEYKGEDAPCKIPFSTEDGQGKAHNGRLITANPVVTKKEEPVNIEAEPPFG
ESNIVIGIGDKALKINWYRKGSSIGK
DENV-4:
(SEQ ID NO: 26)
MRCVGVGNRDFVEGVSGGAWVDLVLEHGGCVTTMAQGKPTLDFELIKTTAKEVALLRTYCIE
ASISNITTATRCPTQGEPYLKEEQDQQYICRRDVVDRGWGNGCGLFGKGGVVTCAKFSCSGK
ITGNLVQIENLEYTVVVTVHNGDTHAVGNDISNHGVTATITPRSPSVEVKLPDYGELTLDCE
PRSGIDENEMILMKMKKKTWLVHKQWFLDLPLPWAAGADTSEVHWNYKERMVTFKVPHAKRQ
DVTVLGSQEGAMHSALTGATEVDSGDGNHMFAGHLKCKVRMEKLRIKGMSYTMCSGKFSIDK
EMAETQHGTTVVKVKYEGAGAPCKVPIEIRDVNKEKVVGRIISSTPFAEYTNSVTNIELEPP
FGDSYIVIGVGDSALTLHWFRKGSSIGK
JEV:
(SEQ ID NO: 27)
FNCLGMGNRDFIEGASGATWVDLVLEGDSCLTIMANDKPTLDVRMINIEASQLAEVRSYCYH
ASVTDISTVARCPTTGEAHNEKRADSSYVCKQGFTDRGWGNGCGLFGKGSIDTCAKFSCTSK
AIGRTIQPENIKYEVGIFVHGTTTSENHGNYSAQVGASQAAKFTVTPNAPSITLKLGDYGEV
TLDCEPRSGLNTEAFYVMTVGSKSFLVHREWFHDLALPWTSPSSTAWRNRELLMEFEEAHAT
KQSVVALGSQEGGLHQALAGAIVVEYSSSVKLTSGHLKCRLKMDKLALKGTTYGMCTEKFSF
AKNPADTGHGTVVIELSYSGSDGPCKIPIASVASLNDMTPVGRLVTVNPFVATSSANSKVLV
EMEPPFGDSYIVVGRGDKQINHHWHKAGSTLGK
ZIKV:
(SEQ ID NO: 28)
IRCIGVSNRDFVEGMSGGTWVDIVLEHGGCVTVMAQDKPTVDIELVTTTVSNMAEVRSYCYE
ASISDMASDSRCPTQGEAYLDKQSDTQYVCKRTLVDRGWGNGCGLFGKGSLVTCAKFACSKK
MTGKSIQPENLEYRIMLSVHGSQHSGMIVNDTGHETDENRAKVEITPNSPRAEATLGGFGSL
GLDCEPRTGLDESDLYYLTMNNKHWLVHKEWFHDIPLPWHAGADTGTPHWNNKEALVEFKDA
HAKRQTVVVLGSQEGAVHTALAGALEAEMDGAKGRLSSGHLKCRLKMDKLRLKGVSYSLCTA
AFTFTKIPAETLHGTVTVEVQYAGTDGPCKVPAQMAVDMQTLTPVGRLITANPVITESTENS
KMMLELDPPFGDSYIVIGVGEKKITHHWHRSGSTIGK

In an embodiment, the neutralizing antibody is a monoclonal antibody.

The term “monoclonal antibody” refers to an antibody that originates from a highly uniform group of antibodies, meaning each antibody in this group is identical except for minor natural variations that may occur in minimal quantities. Therefore, “monoclonal” denotes that these antibodies are not a mixture of different antibodies. Unlike “polyclonal antibody” mixtures, which contain various antibodies targeting different parts of antigens (i.e. epitopes), every monoclonal antibody in a given preparation targets a unique part of the antigen. The monoclonal antibody does not include impurities from other antibodies. The term “monoclonal” does not imply a specific method fro produing said antibody.

In an embodiment, the neutralizing antibody is a full-length antibody or an antigen binding fragment thereof.

In an embodiment, the full-length antibody includes an IgG molecule.

The term “full-length antibody” refers to an antibody having a structure substantially similar to a native antibody structure or having heavy chains that contain an Fc region as defined herein.

The term “antibody fragment” refers to a portion of a full-length antibody which is capable of binding the same antigen as the full-length antibody. Examples of antibody fragments include, but are not limited to, Fv, Fab, Fab′, Fab′-SH, F(ab′)₂; diabodies; linear antibodies; monovalent, or single-armed antibodies; single-chain antibody molecules (e.g., scFv); and multispecific antibodies formed from antibody fragments.

In an embodiment, the antigen binding fragment of the neutralizing antibody is selected from the group consisting of F(ab′)2, Fab′, Fab, Fv, single domain antibody (VHH), and scFv.

The term “F(ab′)2” refers to a pair of Fab fragments which are generally covalently linked near their carboxy termini by hinge cysteines between them.

The term “Fab′” refers to an antibody fragment that contains the Fab fragment with a free sulfhydryl group on CH1. It may be alkylated or conjugated with an enzyme, toxin, or other protein of interest.

The term “Fab” refers to an antibody fragment that contains the V_{H}, CH1 and V_{L}, C_{L}regions, linked by an intramolecular disulfide bond.

The term “Fv” refers to an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the VH-VL dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

The term “single domain antibody (VHH)” refers to a fragment of an antibody consisting of the variable domain of the heavy chain (V_{H}). Canonical antibodies include two heavy chains and two light chains, while single-domain antibodies only have one heavy chain.

The terms “Single-chain Fv” or “scFv” refer to antibody fragments including the V_{H} and V_{L} domains of an antibody, wherein these domains are present in a single polypeptide chain. Generally, an Fv polypeptide further includes a polypeptide linker between the V_{H} and V_{L} domains which enables the scFv to form the desired antigen binding structure.

The present disclosure also provides a method for producing a neutralizing antibody, including: (a) providing a mammal animal; (b) delivering an antigen to the mammal animal, wherein the antigen includes Japanese encephalitis virus (JEV) antigen, Dengue fever virus (DENV) antigen, and/or a combination thereof; (c) selecting a B cell that expresses the neutralizing antibody from the mammal animal, wherein the neutralizing antibody is capable of neutralizing Zika virus (ZIKV); and (d) obtaining the neutralizing antibody from the B cell.

In an embodiment, the mammal animal includes mouse, rat, rabbit, monkey, chimpanzee, and/or human. In a preferred embodiment, the mammal animal is non-human mammal animal.

The term “antigen” refers to a molecule that contains one or more epitopes such as a membrane protein or partial peptides thereof that causes a disease in a subject. An epitope is the specific site of the antigen which binds to a T-cell receptor or specific antibody, and typically comprises about 3 amino acid residues to about 20 amino acid residues. The term antigen refers to killed, attenuated or inactivated bacteria or viruses. The term antigen also refers to antibodies, such as anti-idiotype antibodies or fragments thereof, and to synthetic peptide mimotopes that can mimic an antigen or epitope. In a preferred embodiment, the antigen disclosed in the present disclosure is derived from the flaviviruses.

In an embodiment, in the step (b) of the aforementioned method, the JEV antigen is delivered prior to the DENV antigen to the mammal animal.

The antigen may be delivered to the animal in a sequential manner. For instance, the JEV antigen is initially delivered to the mammal animal (e.g., to infect mammal animal), and once the animal has developed immunity to the JEV antigen, the DENV antigen is then delivered to the mammal animal.

The methods of antigen delivery include the infection of mammals by complete flaviviruses through various transmission types, such as airborne, droplet, direct or indirect physical contact, and fecal-oral routes; or the administration of flaviviruses fragments via methods like intramuscular, intravenous, and intraperitoneal injections, as well as oral, rectal, vaginal, sublingual, and nasal routes.

In an embodiment, the antigen is delivered at levels sufficient to induce an immune response of the mammal animal.

The “immune response” refers to the immune system's reaction in vertebrates to substances recognized as foreign or antigenic. Examples include the activation of cytotoxic T lymphocytes (CTLs), particularly through the specific triggering of CD8+ CTLs, the activities of helper T-cells which involve T-cell proliferation and the release of cytokines, as well as the responses of B-cells, including the production of antibodies.

The “at levels sufficient to induce an immune response” for the antigen may vary from the subject to the subject, depending on the species, age, and general condition of the subject, the stage of the disease, the particular pharmaceutical mixture, its mode of administration, and the like.

The amount of flaviviruses antigen is selected to allow to induce an appropriate immune response with or without significant, adverse side effects. Generally, it is expected that each dose will comprise 0.1-100 μg of polysaccharide, about 0.1-50 μg, about 0.1-10 μg, or about 1-5 μg. Optimal amounts of components for a particular antigen can be ascertained by standard studies involving observation of appropriate immune responses in subjects.

In an embodiment, the JEV antigen includes JEV and/or JEV vaccine. In a preferred embodiment, the JEV vaccine includes inactivated Vero cell culture derived JE vaccine (JE-VC), inactivated mouse brain derived JE vaccine (JE-MB), primary hamster kidney cell derived, attenuated JE vaccine, and/or live attenuated chimeric JE vaccine.

The “JEV antigen” refers to any molecular component of the JEV capable of inducing an immune response in mammals, including the entire JEV of all genotypes (I, II, III, IV, V), recombinant or naturally occurring peptides derived from JEV, and commercial JEV vaccines such as IC51/IXIARO, JE-VAX, SA 14-14-2, and IMOJEV.

In an embodiment, the DENV antigen includes DENV vaccine, DENV serotype 1 (DENV-1), DENV serotype 2 (DENV-2), DENV serotype 3 (DENV-3), and/or DENV serotype 4 (DENV-4). In a preferred embodiment, the DENV vaccine comprises live attenuated dengue vaccine.

The “DENV antigen” refers to any molecular component of the DENV capable of inducing an immune response in mammals, including the entire DENV of all genotypes (DENV-1, DENV-2, DENV-3, DENV-4), recombinant or naturally occurring peptides derived from JEV, and commercial DENV vaccines such as Dengvaxia, TV003/TV005, and TAK-003 (DENVax).

In an embodiment, in the step (d) of the aforementioned method, the neutralizing antibody is obtained from the B cell, or is obtained by expressing an isolated DNA encoding the neutralizing antibody derived from the B cell.

After repeated immunization, the B cell secretes high levels of antibodies into the blood, including the neutralizing antibody. It is then purified by the methods in the art, such as protein A or G affinity chromatography, or antigen-affinity purification. Preferably, the gene encoding the neutralizing antibody (e.g. DNA) is isolated from the B cell by gene amplification method (e.g. PCR). This gene is then cloned into an expression vector to produce the neutralizing antibody.

In an embodiment, the isolated DNA encodes a full-length antibody of the neutralizing antibody or an antigen binding fragment thereof. In a preferred embodiment, the full-length antibody includes an IgG molecule.

The present disclosure also provides a pharmaceutical composition including the any one of aforementioned neutralizing antibody, optionally together with a pharmaceutically acceptable carrier, diluent or excipient.

The term “pharmaceutical composition” refers to a preparation in a form that allows the biological activity of the active ingredient(s) to be effective, and which contain no additional components which are toxic to the subjects to which the composition is administered. A pharmaceutical composition may include one or more active agents. For example, a pharmaceutical composition may include the neutralizing antibody as the sole active agent of the formulation or may include the neutralizing antibody and one or more additional active agents, an immune activator, or an inhibitor of an immune checkpoint molecule.

The term “pharmaceutically acceptable carrier” refers to an ingredient in a pharmaceutical composition, other than an active ingredient, which is nontoxic to the subject to whom it is administered. A pharmaceutically acceptable carrier includes a buffer, excipient, stabilizer, or preservative.

The present disclosure also provides a method of treating or preventing a flaviviruses infection in a subject, including administering to the subject an effective amount of the aforementioned pharmaceutical composition under conditions effective to neutralize the flaviviruses, thereby treating or preventing the flaviviruses infection in the subject.

There are mainly two types of flaviviruses infection: acute flavivirus infection and convalescent flavivirus infection. The “acute flavivirus infection” refers to a flavivirus infection that is characterized by rapid onset of disease, a relatively brief period of symptoms, and resolution within days. A rapid flavivirus infection is usually accompanied by early production of infectious visions and elimination of infection by the host immune system. Within an acute flavivirus infection Ab titers in body fluids are high compared to a convalescent virus infection. The flavivirus within that context may be a DENV (acute DENV infection), ZIKV (“acute ZIKV infection”), and JEV (acute JEV infection). An “acute flavivirus infection” may refer to the period of viremia. The term “convalescent flavivirus infection” refers to a flavivirus infection that has been eliminated by the host immune system. A characteristic of a convalescent flavivirus infection is the existence of memory B-cells encoding for Abs against the flavivirus that has caused the infection. Within a convalescent flavivirus infection Ab titers in body fluids are low compared to an acute flavivirus infection. The flavivirus within that context may be a DENV (acute DENV infection), ZIKV (“acute ZIKV infection”), and JEV (acute JEV infection). A “convalescent flavivirus infection” may refer to the period after viremia.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having inflammatory diseases, autoimmune diseases, cancer, infectious diseases or other disorders requiring modulation of the immune response. A subject having a target disease or disorder can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors for that disease/disorder.

The methods and compositions described herein may be used to treat flaviviruses infection. Exemplary flaviviruses infection are Dengue fever, West Nile virus, Yellow fever, Japanese encephalitis, Zika virus, Kyasanur Forest disease, Omsk hemorrhagic fever, Alkhurma hemorrhagic fever. The symptom caused by the above diseases includes fever, headache, muscle aches, weakness, nausea, vomiting, or diarrhea, skin rash, swollen lymph glands, vision problems, confusion, and/or coma.

The present disclosure also provides a use of the any one of aforementioned neutralizing antibody for the manufacture of a medicament for treating or preventing a flaviviruses infection in a subject.

The following representative examples illustrate various features and embodiments of the disclosure, which are intended to be illustrative and not limiting. Those skilled in the art will readily appreciate that the specific examples are only illustrative of the invention as described more fully in the subsequent claims. Every embodiment and feature described in the present application should be understood to be interchangeable and combinable with every embodiment contained within.

### Examples

### Methods

### Ethics Statement, Patient Acquisition, and Isolation of Human Peripheral Blood B Cells

The use of human blood specimens was reviewed and approved by the Institutional Review Board (IRB) of Kaohsiung Medical University (IRB No. KMUHIRB-E (II)-20180092). The donors signed a written informed consent before any data collection. All data were anonymized.

All subjects enrolled in this study were from a dengue cohort in 2014-2015, during the two largest DENV-1 and DENV-2 outbreaks in southern Taiwan. The archived febrile patients' plasma samples were obtained from the dengue clinical cohort study of Kaohsiung Medical University Hospital (KMUH) in southern Taiwan through an ongoing study that enrolled subjects with febrile symptoms suspected of dengue viral infection during the acute phase (i.e., 2 weeks post-infection (poi)). The plasma samples comprised 60 dengue-confirmed individuals and 80 dengue-negative controls, also termed healthy in this study, based on the clinical and laboratory diagnosis upon collection. The nationwide JEV pediatric vaccination program in Taiwan has been implemented since 1968. To distinguish between individuals immune to JEV due to vaccination or potential natural infection, the study population was divided into two age groups, namely 20-30 years old (post-JEV vaccination program) and 50-70 years old (pre-JEV vaccination program). Laboratory confirmation of DENV infection consisted of quantitative reverse transcription-polymerase chain reaction (qRT-PCR) and virus isolation. Duplicate blood samples at the time of symptom onset were sent to Taiwan Centers for Disease Control for RT-PCR screening for arbovirus infection using flavivirus consensus and chikungunya virus-specific primers. DENV, JEV, and ZIKV infections are notifiable and reportable infectious diseases in Taiwan. All subjects were confirmed with DENV infection and no autochthonous transmission of Zika virus infections has been observed. Epidemiological data were also obtained, including demographics, travel, and vaccination histories. All subjects were not vaccinated against YFV. De-identified serum samples were evaluated for neutralizing antibodies against JEV, DENV-1 to -4, ZIKV, and YFV.

To comprehensively determine the impact of JEV and DENV immunity on the ZIKV-specific antibody response, the inventors further classified the donors into four groups: (1) JEV^{pos}DENV^{high}, (2) JEV^{pos}DENV^{low}, (3) JEV^{neg}DENV^{high}, and (4) JEV^{neg}DENV^{low} by dichotomizing those with JEV FRμNT90 titers ≥10 as JEV^{pos} or <10 as JEV^{neg}, and those with DENV FRμNT90 titers ≥80 as DENV^{high} or DENV^{low} (FRμNT90<80). Previous criteria on serum-neutralizing antibodies (NT50)>30 indicate primary dengue infections, while the neutralizing antibodies (NT50) among homotypic and heterotypic dengue-infected individuals at the acute phase of infection ranged from 1:40 to <1:1,280. Considering the epidemiology of dengue infections in Taiwan and the more stringent threshold of neutralization (NT90) the inventors previously set, the inventors considered serum neutralizing (NT90) antibodies ≥80 as evidence of durable dengue titers (DENV^{high}) or <80 for confirmed yet low dengue titers (DENV^{low}). In addition, JEV-infected individuals with neutralizing antibodies ≥10 were considered seropositive and protective. Since most primary or dengue-infected individuals also showed lower cross-neutralization titers against ZIKV than the infecting DENV or heterologous DENV serotypes, the inventors further classified the varying ZIKV neutralization titers from DF individuals as ZIKV^{pos} (ZIKV FRμNT90≥20) and ZIKV^{neg} (ZIKV FRμNT90<20).

Whole-blood samples were collected into EDTA precoated blood collection tubes (Becton Dickinson) from one of the recalled dengue-immune individuals (KH1891) at late convalescence (>18 months after recovery). Human peripheral blood mononuclear cells (PBMCs) were isolated using the Ficoll-Paque medium (GE Healthcare, Uppsala, SWE) and density gradient method under sterile conditions for sorting.

### Cells, Antibodies, and Virus Propagations

Vero (CRL 1587; ATCC, Manassas, VA, USA) and HEK293T (CRL-3216; ATTC, Manassas, VA, USA) cells were grown at 37° C. with 5% CO₂ in Dulbecco's modified Eagle's minimal essential medium (DMEM; Gibco, Grand Island, NY, USA) supplemented with 10% heat-inactivated fetal bovine serum (FBS), 110 mg/L sodium pyruvate, 0.1 mM nonessential amino acids (Gibco, Life Technologies, Grand Island, NY, USA), 2 mM L-glutamine, 20 mL/L 7.5% NaHCO₃, 100 IU/mL penicillin and 100 μg/mL streptomycin. All the cells used were free of mycoplasma contamination and checked regularly by following the commercial protocol (InvivoGen, Hong Kong).

Serotype-specific anti-DENV VLP, anti-JEV VLP, and anti-ZIKV VLP mouse hyperimmune ascitic fluid (MHIAF) or sera were either in-house prepared or kindly provided by Dr G.-J. Chang (recently retired from Division of Vector-borne Diseases, Centers for Disease Control and Prevention, DVBD-CDC, Fort Collins, CO, USA).

All flaviviruses were propagated on Vero cells using DMEM supplemented with 2% heat-inactivated FBS, 100 IU/mL penicillin and 100 μg/mL streptomycin for 5-7 days. Infected cell culture supernatants were harvested, clarified by centrifugation at 2000×g, and stored in aliquots at −80° C. The virus strains used in this study were DENV-1 Hawaii, DENV-1 BC 245/97, DENV-2 16681, DENV-2 94 Puerto Rico (PR), DENV-3 H87, DENV-3 KH9800235, DENV-4 BC 71/94, DENV-4 H241, JEV CJN-S1, YFV-17D, ZIKV-MR 766, and ZIKV-PRVABC59. All prototype DENV serotypes 1-4, JEV, and ZIKV virus strains were provided by Dr G.-J. Chang (Diagnostic and Reference Laboratory, DVBD-CDC, Fort Collins, CO, USA), while clinical isolate DENV-3 KH9800235 was provided by one of the co-authors, Dr Y.-H. Chen.

### Focus-Reduction Microneutralization Test (FRμNT)

To measure the in vitro neutralization activity of the human and murine polyclonal antibodies with DENVs, ZIKV, JEV, and YFV, a focus-reduction microneutralization test (FRμNT) in Vero cells was performed. All human and murine polyclonal sera were heat-inactivated at 56° C. for 30 min prior to use in the experiments. Each purified human monoclonal antibody was serially diluted starting at 20 μg/mL. For human polyclonal sera (starting with 10-fold dilution), two-fold serial dilutions were incubated with equimolar concentrations (200 ffu/well) of each of the flavivirus stocks diluted in serum-free DMEM and incubated at 37° C. in 5% CO₂ for 1 h before adding to Vero cells seeded at 180,000 cells/mL the day prior. After 1 h adsorption, the cells were overlayed with 1% carboxymethylcellulose (Sigma-Aldrich Inc., St. Louis, MO, USA) in DMEM with 2% FBS. Immunostaining was performed by adding serotype-specific MHIAF. The infection foci were visualized using a peroxidase substrate kit, Vector VIP SK-4600 (Vector Laboratories, Inc., Burlingame, CA, USA), following the manufacturer's instructions. Each plate included infected and uninfected cell controls and naïve human serum. The microneutralization antibody titers of each serum against each virus were defined as the reciprocal of the antibody dilution with the percentage of reduction of virus infectivity relative to the challenge virus dose for each plate. The obtained actual foci count from cells infected with the specific virus (DENV-1 to -4, JEV, or ZIKV) in the absence of plasma were used as virus control, representing 100% infection. The inventors performed the analysis at FRμNT50, 75, and 90 as 50%, 75%, or 90% reduction, respectively, using four-parameter nonlinear regression (version 9.5.1, GraphPad Software Inc.) and found that reporting the more stringent FRμNT90 showed a more conservative threshold of virus neutralization titers and, consequently, a better grasp of exposure history and breadth of neutralization to aid the serum classification as primary or repeat DENV.

### Focus-Reduction Microneutralization (FRμNT)-ELISA

The inventors established an in-house ELISA-based microneutralization test for high-throughput measurement of virus neutralization by detecting the optical density (OD) signal from infected cells. This method was used for virus neutralization screening in hybridoma, small-scale and large-scale expressed and purified human monoclonal antibodies. The standard FRμNT was performed as described above with modifications. Specifically, virus-infected plates were incubated for 72 h at 37° C. with 5% CO₂, fixed with ice-cold 75% acetone in 1×PBS for 30 min, and air dried. Each plate included infected and uninfected cell controls and naïve human serum or purified isotype control antibodies. The ELISA was performed by adding 100 μL/well of unpurified, equimolar concentrations of pan-flavivirus anti-E murine MAb FL0231 and anti-NS1 murine MAb mFL0221, which were kind gifts from Dr L.-K. Chen (Tzu Chi University Hospital, Hualien, Taiwan). After incubating for 1 h at 37° C., the plates were washed five times with 1×PBS/0.1% Tween-20 washing buffer. Peroxidase-conjugated goat anti-mouse IgG (Jackson ImmunoResearch Laboratories Inc., West Grove, PA, USA) diluted at 1:5,000 in 5% skimmed milk-1×PBS (0.1% Tween-20) was added to the wells, and the plates were incubated at 37° C. for 1 h. Finally, the plates were washed ten times, and the bound conjugate was detected with TMB substrate by measuring the absorbance at 450 nm/630 nm. All the optical density (OD) readings were normalized by subtracting the mean OD of uninfected controls from the mean OD of virus controls or the mean OD of samples. The cut-off value (for each plate) for the virus back titration is the mean normalized virus controls as a function of the working virus dilution, determined by applying a three-parameter nonlinear curve fit. The microneutralization antibody titers of each serum or purified monoclonal antibodies against each virus were defined as the reciprocal of the antibody dilution that reduced virus infectivity (and consequently, color development) by 50% (FRμNT50) or 90% (FRμNT90) relative to the infecting virus with control antibody or serum for each plate. The obtained relative optical density (OD) values were normalized to those derived from cells infected with the specific virus (DENV-1 to -4, JEV, or ZIKV) without monoclonal antibodies. The half-maximal inhibitory concentration for monoclonal antibodies (IC50) was determined using four-parameter nonlinear regression (version 9.5.1, GraphPad Software Inc.). All data were log-transformed for analysis, processed, and graphed by GraphPad Prism (version 9.5.1, GraphPad Software Inc.).

Single B Cell Sorting, Co-Cultivation with EL4-B5 Cells, and B Cell Screening by ELISA

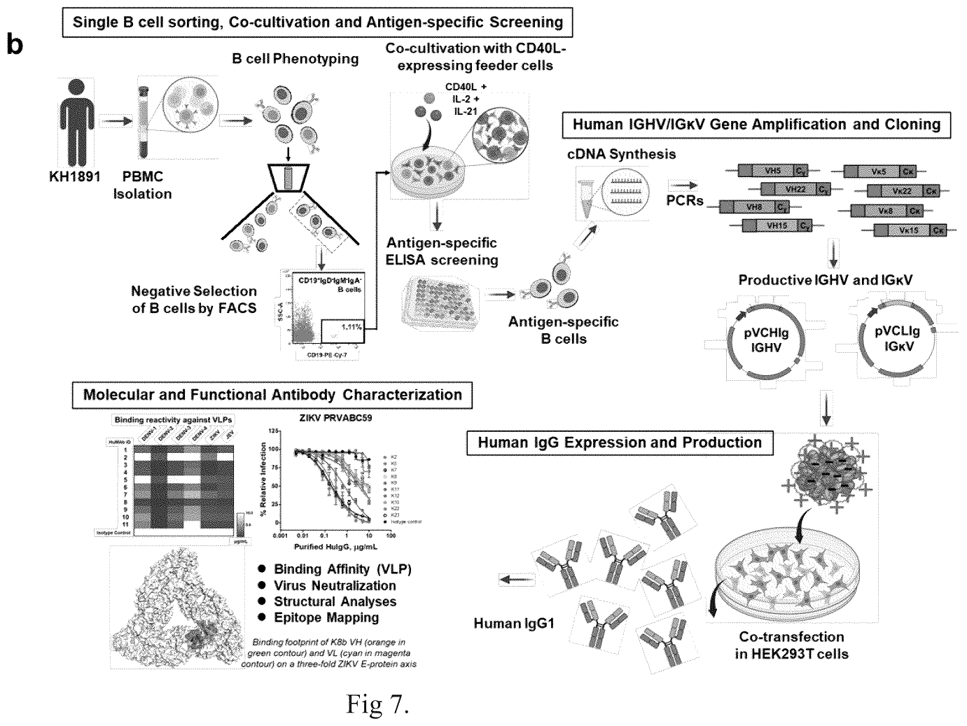

Freshly isolated PBMCs from KH1891 were stained on ice with fluorescently-conjugated anti-human antibodies: CD19-PE-Cy7 (Mat. No. 557835, BD Biosciences Pharmingen™, San Diego, CA, USA), IgM-PE (Code No., 709-116-073, Jackson ImmunoResearch Laboratories Inc., West Grove PA, USA), IgA-APC (Code No., 109-135-011, Jackson ImmunoResearch Laboratories Inc., West Grove, PA, USA), and IgD-FITC (Mat. No., 555778, BD Biosciences Pharmingen™, San Diego, CA, USA) to select CD19⁺IgM⁻IgA⁻IgD⁻ B cell subpopulation (FIG. 7). Single cells were sorted according to % yield excluding cell duplets and gated on the live CD19⁺IgM⁻IgD⁻IgA⁻ B cells to determine the target fraction described previously; compensation controls comprised positively stained and negative or unstained cell populations. Sorted B cells were immediately co-cultivated with irradiated EL4-B5 feeder cells expressing CD40L at a seeding density of 3 B cells per well in 384-well plates in the presence of IL-21 and IL-2 in complete Iscove's modified Dulbecco medium (IMDM, Thermo Fisher Scientific, Inc., Rockford, IL, USA) incubated at 37° C. with 5% CO₂ and left undisturbed for 2 weeks. After 13 days of cultivation, 40 μL of the culture supernatant from each well was collected and independently screened for binding to the cell culture supernatant containing ZIKV particles by antigen-capture ELISA. ELISA readings were reported according to the average positive-to-negative (P/N) ratio at A_{450nm/630nm} of each sample (reference at 630 nm). The P/N ratio was computed after normalization with the negative control (NC_{cell}) obtained from the cell culture supernatant with EL4-B5 cells. Purified total human IgG controls were included per plate and were purified from PV10 (PC) and TW2 (NC_{TW2}) plasma by affinity chromatography using 1 mL HiTrap Protein G HP column (GE Healthcare, Uppsala, SWE) and 0.2 μm filtered pH-adjusted buffers. Positively screened B cells were ranked from the highest to lowest ELISA P/N values, which was the basis for prioritizing human monoclonal antibody expression.

### IgG1, IgG3, F(Ab), and F(Ab′)₂ Plasmid Construction, Human Ig Gene Amplification, and Expression Vector Cloning

In preparation for recombinant immunoglobulin G1 (IgG1) antibody production, in-house expression plasmids pVCHIg-hG1 and pVCLIg-hx were developed for independent cloning of the human Ig variable heavy (VH) and light (VL) chain genes, respectively. Transcription was controlled by the human cytomegalovirus early gene promoter (^{P}CMV). Both expression plasmids contain the bovine growth hormone poly(A) signal (BGH pA) and the Kanamycin resistance (Kan^{R}) cassette. To facilitate the linearization of the expression vectors, AgeI and SalI were added at the respective 5′- and 3′-ends of the pVCHIg-hG1-vector, while the AgeI and BsiWI sites were introduced at the 5′- and 3′-ends of pVCLIg-hκ expression vectors, respectively.

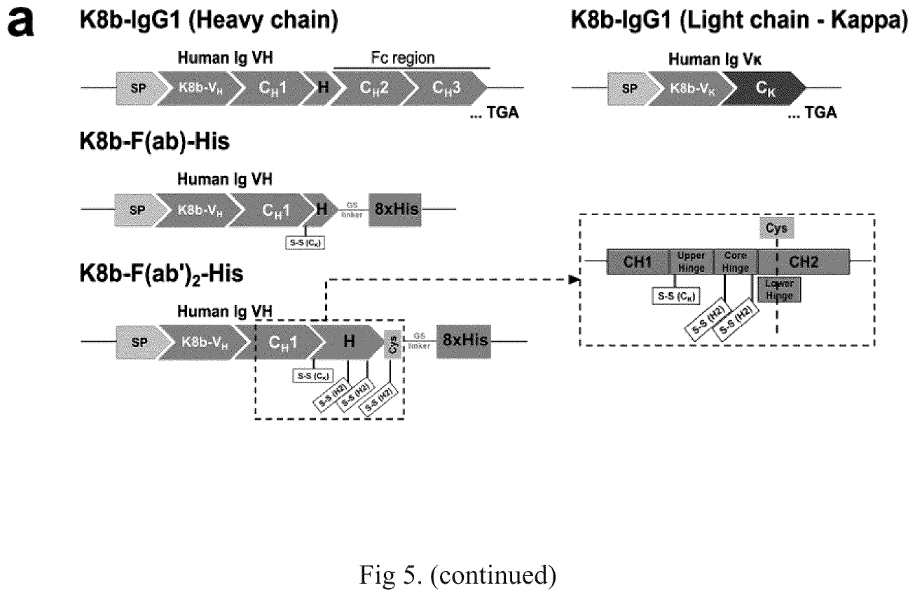

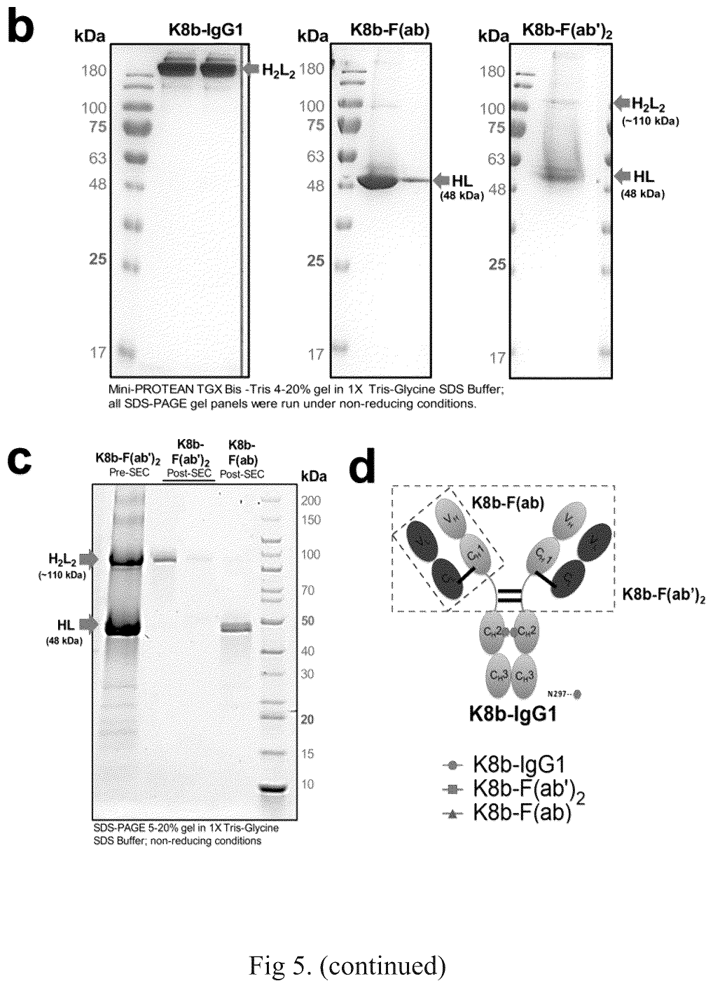

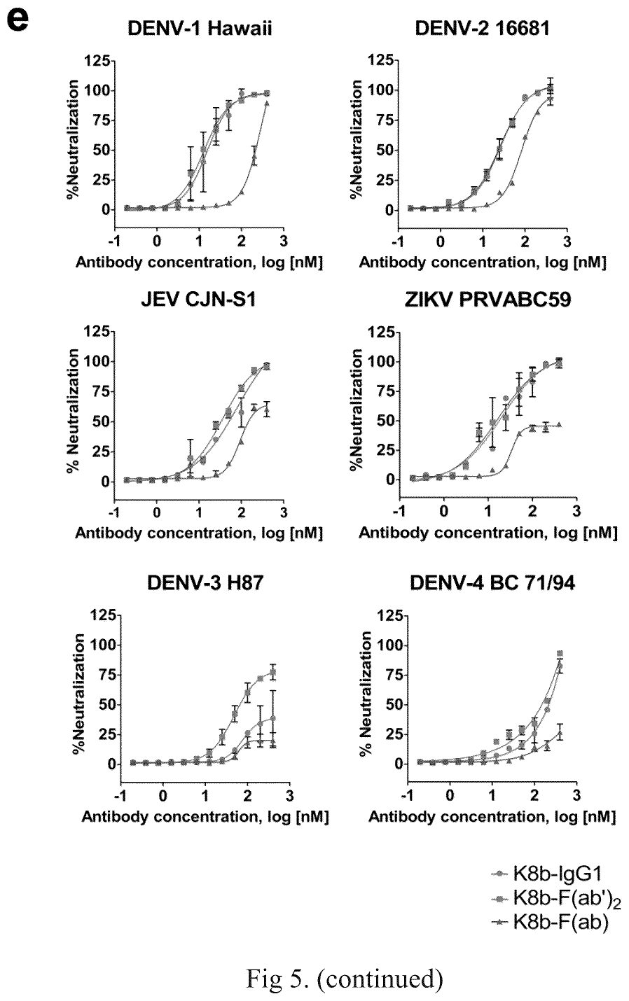

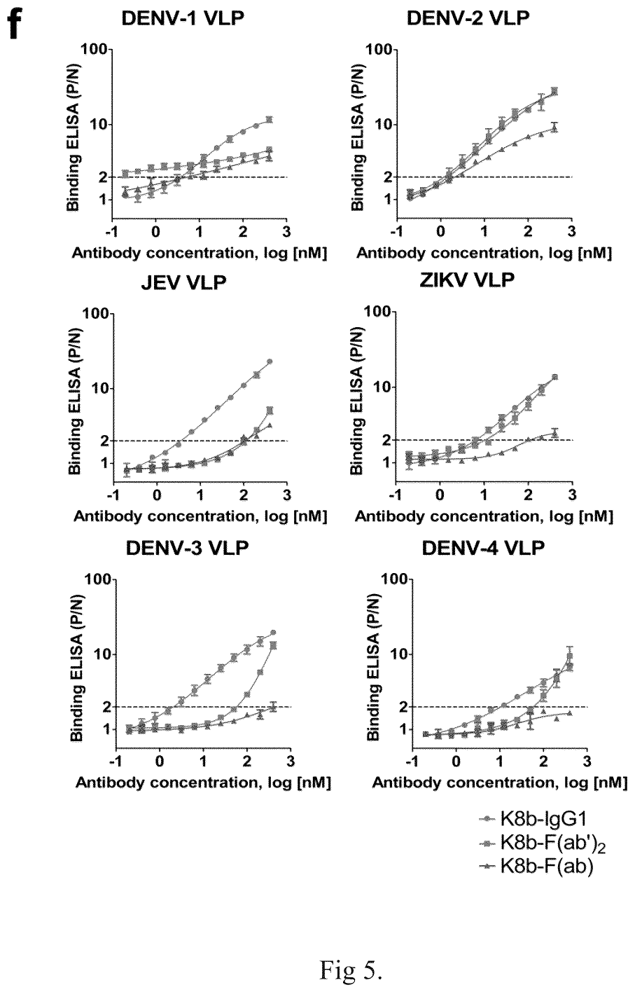

To construct the K8b-F(ab) expression plasmids, the full-length K8b VH region with the human constant heavy chain 1 (hCH1) cassette was pre-amplified, including the overlapping sequences encoding the linker and 8×-His residues at the 3′-end. For the F(ab′)₂ expression vector, the amplification of the insert was performed from the hCH1 cassette until the leucine (L117) residue located at the 5′-end of the hCH2 cassette, which is five nucleotides downstream of the third conserved cysteine (Cys112) residue for heavy chain dimerization. The F(ab) and F(ab′)₂ expression plasmids were modified to contain an 8×-Histidine tag at the carboxyterminal segment with QuikChange II site-directed mutagenesis kit (Stratagene, La Jolla, CA, USA) (FIG. 5a). An additional cysteine residue (Cys118) was inserted in between the L117 and the Histidine tag sequence to enhance the thermostability of F(ab′)₂ molecules (FIG. 5a). An inverse PCR was set up to amplify necessary regulatory elements in the pVCHIg-hG1 vector backbone. The resulting PCR fragments shared complementary ends and were joined by subsequent sequence and ligation-independent cloning (SLIC). PCRs were carried out with the Phusion High Fidelity DNA Polymerase according to the manufacturer's protocol (New England Biolabs, Inc., Ipswich, MA, USA). The generated plasmids were recovered by a standard transformation protocol into chemically competent E. coli. Standard transformation was performed with the addition of pre-warmed SOC media after incubation at 37° C. for 1 h with shaking at 200 rpm, and 100 μL was plated onto Luria-Bertani (LB)-agar Petri dishes containing 100 μg/mL Kanamycin. Sequences were verified by sequencing and herein termed pVCHIg-K8b-F(ab′)₂-His and pVCHIg-K8b-F(ab)-His. A similar inverse PCR was set up to amplify the variable region expressing K8b and the regulatory elements in the pVCHIg-hG1 vector backbone without the human Ig constant gamma 1 (Igγ1) region, while an independent PCR using SLIC-adapted primers was performed to amplify the human Ig γ3 region from pFUSE-CHIg-hG3 (allele 01) including its four hinge domains (InvivoGen, Hong Kong). All the expression plasmids contain the same promoter, enhancer, and selection marker gene cassettes. Primers used for constructing pVCHIg-K8b-IgG1, pVCHIg-K8b-F(ab′)₂-His, pVCHIg-K8b-F(ab)-His, pVCHIg-K8b-IgG3, and pVCLIg-hK-K8b are summarized in Table 2.

TABLE 2
Oligonucleotide primers used for the generation of various
in-house antibody expression vectors.

| | | | |
|---|---|---|---|
| Template DNA | Target Fragment | Primer Name | Primer sequence (5′ ---> 3′) |

Generation of pVCHIg-K8b-IgG1
K8b IGHV insert

| | | | |
|---|---|---|---|
| pCDNA K8b | K8b IGHV | SP K8b | 5′ -- ccttgtccttgttttaaccggt |
| | | IGHV FP | gtccagtgtCAGGTGCAGCTGGTGCAGC |
| | | | CTGGAGCAG --- 3′ |
| | | | (SEQ ID NO: 29) |
| | | hG1 | 5′ --- ccgatgggcccttggtcgacg |
| | | CH1_SalI_K | cGGAGCTCACTGTCACGCTGGTGCCCTG |
| | | 8b RP | GC --- 3′ |
| | | | (SEQ ID NO: 30) |

pVCHIg-hG1 vector backbone

| | | | |
|---|---|---|---|
| pVCHIg-hG1 | pVCHIg-hG1 | K8b | 5′ --- GCCAGGGCACCAGCGTGACAG |
| | | CH3_hG1 | TGAGCTCCgcgtcgaccaagggcccatc |
| | | CH1 FP | ggtcttccc --- 3′ |
| | | | (SEQ ID NO: 31) |
| | | pVCHIg- | 5′ --- CAGGCTGCACCAGCTGCACCT |
| | | hG1SP_K8b | Gacactggacaccggttaaaacaaggac |
| | | RP | aag --- 3′ |
| | | | (SEQ ID NO: 32) |

Generation of pVCHIg-K8b-F(ab)
K8b IGHV insert

| | | | |
|---|---|---|---|
| pVCHIg-K8b- | K8b IGHV | SP inframe | 5′ --- cttcgggttcagcttgatttt |
| hG1 | insert + | FP | ccttgtccttgttttaaccgg --- 3′ |
| | 8xHis | | (SEQ ID NO: 33) |
| | | hG1 | 5′ --- gatgatgatgatgggacccag |
| | | disulfide | ttttgtcacaagatttgggctcaacttt |
| | | 8x His RP | cttgtccaccttggtg --- 3′ |
| | | | (SEQ ID NO: 34) |

pVCHIg-hG1-F(ab) vector backbone

| | | | |
|---|---|---|---|
| pVCHIg-hG1 | GS Linker- | pVCHIg_hG1 | 5′ --- gggtcccatcatcatcatcat |
| | 8xHis-SP | F(ab) | catcatcattgagcggccgctcgagtct |
| | | 8xHis FP | ag --- 3′ |
| | | | (SEQ ID NO: 35) |
| | | pVCHIg- | 5′ --- ATTAGAATTCacactggacac |
| | | mG2_v2 | cggttaaaacaaggacaagg --- 3′ |
| | | AgeI RP | (SEQ ID NO: 36) |

Generation of pVCHIg-K8b-F(ab′)₂
K8b IGHV insert

| | | | |
|---|---|---|---|
| pVCHIg-K8b- | K8b IGHV | SP inframe | 5′ --- cttcgggttcagcttgatttt |
| IgG1 | insert + | FP | ccttgtccttgttttaaccgg --- 3′ |
| | 3rd Cys + | | (SEQ ID NO: 33) |
| | 8x His | | |
| | | hG1 CH1 | 5′ --- gatgatgatgatgatgatgat |
| | | Cys3 8xHis | gggacccgcagagttcaggtgctgggca |
| | | RP | cggtgggcatgtg --- 3′ |
| | | | (SEQ ID NO: 37) |

pVCHIg-hG1-F(ab′)2 vector backbone

| | | | |
|---|---|---|---|
| PVCHig-hG1- | CHIg + | 3rdCys_ | 5′ --- cacacatgcccaccgtgccca |
| F(ab) | 3rdCys + | 8xHis FP | gcacctgaactctgcgggtcccatcatc |
| | GSlinker + | | atcatcatcatcatc --- 3′ |
| | 8x His | | (SEQ ID NO: 38) |
| | | pCHIg- | 5′ --- ATTAGAATTCacactggacac |
| | | mG2_v2 | cggttaaaacaaggacaagg --- 3′ |
| | | AgeI RP | (SEQ ID NO: 36) |

Generation of pVCHIg-K8b-IgG3
human IgG3 (hG3) Fc-region including Hinge domains 1-4

| | | | |
|---|---|---|---|
| pFUSEss-CHIg- | CHIg-hG3 | K8b_VH_ | 5′ --- GGCACCAGCGTGACAGTGAGC |
| hG3*01 | CH1 to CH3 | SalI_hG3_ | TCCgcgtcgaccaagggcccatcggtct |
| | | CH1 FP | tcc --- 3′ |
| | | | (SEQ ID NO: 39) |
| | | pVCHIg_hG3 | 5′ --- ctagactcgagcggccgctca |
| | | CH3 RP | tttacccggagacagggagaggc |
| | | | --- 3′ |
| | | | (SEQ ID NO: 40) |

pVCHIg-K8b IGHV backbone with regulatory elements from pVCHIg-hG1

| | | | |
|---|---|---|---|
| pVCHIg-K8b- | pVCHIg-K8b | hG3 | 5′ --- gcctctccctgtctccgggta |
| IgG1 | IGHV insert | CH3_pVCHIg | aatgagcggccgctcgagtctag |
| | | FP | --- 3′ |
| | | | (SEQ ID NO: 41) |
| | | hG3 | 5′ --- ccgatgggcccttggtcgacg |
| | | CH1_SalI_ | cGGAGCTCACTGTCACGCTGGTGCCCTG |
| | | K8b RP | GC --- 3′ |
| | | | (SEQ ID NO: 42) |

Generation of pVCLIg-K8b
K8b IGKV insert

| | | | |
|---|---|---|---|
| pCDNA K8b | K8b IGKV | SP K8b | 5′ --- gtccttgttttaaccggtgtc |
| | | IGKV FP | cagtgtGAAATAGTGATGATGCAGTCTC |
| | | | CACTC --- 3′ |
| | | | (SEQ ID NO: 43) |
| | | hK K8b | 5′ --- gaagacagatggcgccgccac |
| | | IGKV RP | cgtacgTTTGATCTCCACCTTGGTCCCT |
| | | | TGGCCG --- 3′ |
| | | | (SEQ ID NO: 44) |

pVCLIg-hK vector backbone

| | | | |
|---|---|---|---|
| pVCLIg-hK | PVCLIg-hK | K8b IGKV | 5′ --- CGTTCGGCCAAGGGACCAAGG |
| vector | | PVCLIg-hK | TGGAGATCAAAcgtacggtggcggcgcc |
| backbone | | FP | atctg --- 3′ |
| | | | (SEQ ID NO: 45) |
| | | pVCLIg-hK | 5′ --- GTGGAGACTGCATCATCACTA |
| | | SP K8b RP | TTTCacactggacaccggttaaaacaag |
| | | | gacaag --- 3′ |
| | | | (SEQ ID NO: 46) |

Sequences targeting the human Immunoglobulin (Ig) variable (V) genes are highlighted in red and are denoted in upper case format, while the sequences in blue are part of the vector backbone and are in lower case.
IGHV, Immunoglobulin heavy chain;
IGKV, immunoglobulin kappa light chain;
SP, signal peptide;
FP, forward primer;
RP, reverse primer.

Human genes encoding immunoglobulin (Ig) variable heavy (IgHV) and Ig variable kappa (IgκV) chains from ZIKV-reactive B cells were recovered using a two-step RT-PCR strategy. Total RNA was reverse transcribed from lysed B cells, primed using random hexamers (Invitrogen, Carlsbad, CA, USA), and digested with RNAse H at 37° C. for 20 min to remove any excess RNA. Human IgHV and IgκV were amplified independently by two rounds of PCR amplifications using modified primer sets (Table 3). The first-round PCR used forward primers in the heavy and light chain functional leader sequence and reverse primers specific to the constant region of IgHV or IgκV, preserving information about the variable region and the isotype. The second-round PCRs were performed with primers annealing to the 5′-end of the variable (V) genes starting from the framework region 1 (FR1) and their respective reverse primers specific to the joining (J) region of IgHV or IgκV. All primers used in the second-round PCR were designed in preparation for the in vitro assembly using the sequence- and ligation-independent cloning (SLIC) method. SLIC-adapted primers were designed to generate a blunt 5′-end and to contain 20-30 bp sequences complementary to the 5′- or 3′-ends of the expression vector and flanking the human IGHV or IGκV sequences. All PCR reactions were performed with a total volume of 40 μL per sample containing 0.5 μM each of the forward or reverse primers, 300 μM each of the dNTP mix, and 1.25 U One Taq DNA Polymerase (Invitrogen, Carlsbad, CA, USA). All second-round or cloning PCR reactions with gene-specific primers were performed with 3.5 μL of unpurified first PCR products. Each round of PCR was treated as follows: initial denaturation for 30 s at 94° C., then 50 cycles of denaturation at 94° C. for 30 s, annealing at 58° C. for 30 s, and extension at 72° C. for 55 s (1st PCR) or 45 s (2nd PCR), followed by a final extension of 68° C. for 5 min and a cool down to 4′° C. Each of the successfully amplified Ig variable heavy (IGHV) and Kappa (IGκV) genes was visualized by gel electrophoresis on a 2% (wt/vol) agarose gel in preparation for cloning.

TABLE 3
Human monoclonal antibody isolation primers
used in the present disclosure.

| | | SEQ ID |
|---|---|---|
| | Primer sequence (5′ ---> 3′) | NO |
| 1st PCR Primers | | |

Human Heavy chain

| | | |
|---|---|---|
| 5′ L-VH1 | ACAGGTGCCCACTCCCAGGTGCAG | 47 |
| 5′ L-VH3 | AAGGTGTCCAGTGTGARGTGCAG | 48 |
| 5′ L-VH 4/6 | CCCAGATGGGTCCTGTCCCAGGTGCAG | 49 |
| 5′ L-VH 5 | CAAGGAGTCTGTTCCGAGGTGCAG | 50 |
| 3′ Cy CH1 | GGAAGGTGTGCACGCCGCTGGTC | 51 |

Human Kappa Light chain

| | | |
|---|---|---|
| 5′ L VK 1/2 | ATGAGGSTCCCYGCTCAGCTGCTGG | 52 |
| 5′ L VK 4 | ATTTCTCTGTTGCTCTGGATCTCTG | 53 |
| 3′ CK 543-566 | GTTTCTCGTAGTCTGCTTTGCTCA | 54 |
| 2nd PCR Primers | | |

Human Heavy chain: AgeI huVH Forward Primer mix

| | | |
|---|---|---|
| 5′ AgeI huVH1 | CTTGTCCTTGTTTTAACCGGTGTCCAGTGTCAGGTGCA | 55 |
| | GCTGGTGCAG | |
| 5′ AgeI huVH1-2 | CTTGTCCTTGTTTTAACCGGTGTCCAGTGTCAGGTGCA | 56 |
| | GGTGGTGCAG | |
| 5′ AgeI huVH1/5 | CTTGTCCTTGTTTTAACCGGTGTCCAGTGTGAGGTGCA | 57 |
| | GCTGGTGCAG | |
| 5′ AgeI huVH1-18 | CTTGTCCTTGTTTTAACCGGTGTCCAGTGTCAGGTTCA | 58 |
| | GCTGGTGCAG | |
| 5′ AgeI huVH1-24 | CTTGTCCTTGTTTTAACCGGTGTCCAGTGTCAGGTCCA | 59 |
| | GCTGGTACAG | |
| 5′ AgeI huVH3 | CTTGTCCTTGTTTTAACCGGTGTCCAGTGTGAGGTGCA | 60 |
| | GCTGGTGGAG | |
| 5′ AgeI huVH3-23 | CTTGTCCTTGTTTTAACCGGTGTCCAGTGTGAGGTGCA | 61 |
| | GCTGTTGGAG | |
| 5′ AgeI huVH3-33 | CTTGTCCTTGTTTTAACCGGTGTCCAGTGTCAGGTGCA | 62 |
| | GCTGGTGGAG | |
| 5′ AgeI huVH3- | CTTGTCCTTGTTTTAACCGGTGTCCAGTGTGAAGTGCA | 63 |
| 9/30/33 | GCTGGTGGAG | |
| 5′ AgeI huVH4 | CTTGTCCTTGTTTTAACCGGTGTCCAGTGTCAGGTGCA | 64 |
| | GCTGCAGGAG | |
| 5′ AgeI huVH4-34 | CTTGTCCTTGTTTTAACCGGTGTCCAGTGTCAGGTGCA | 65 |
| | GCTACAGCAGTG | |
| 5′ AgeI hu VH 4-39 | CTTGTCCTTGTTTTAACCGGTGTCCAGTGTCAGCTGCA | 66 |
| | GCTGCAGGAG | |
| 5′ AgeI huVH 6-1de | CTTGTCCTTGTTTTAACCGGTGTCCAGTGTCAGGTRCA | 67 |
| | GCTGCAGCAG | |

Human Heavy chain: SalI huJH Reverse Primer mix

| | | |
|---|---|---|
| 3′ SalI huJH | GACCGATGGGCCCTTGGTCGACGCKGARGAGACGGTGA | 68 |
| 1/2/4/5 | CCA | |
| 3′ SalI huJH | GACCGATGGGCCCTTGGTCGACGCTGAGGAGACAGAGA | 69 |
| 1/2/4/5B | CCAG | |
| 3′ SalI huJH 3 | GACCGATGGGCCCTTGGTCGACGCTGAAGAGACGGTGA | 70 |
| | CCATTG | |
| 3′ SalI huJH 4/6 | GACCGATGGGCCCTTGGTCGACGCGGAGCTCACTGTCA | 71 |
| | CGCT | |
| 3′ SalI huJH 6 | GACCGATGGGCCCTTGGTCGACGCTGARGAGACGGTGA | 72 |
| | CCGTG | |
| 3′ SalI huJH 6B | GACCGATGGGCCCTTGGTCGACGCTGAGGAGACGGTGA | 73 |
| | GTGT | |

Human Kappa Light Chain: 5′-AgeI huVK Forward Primer mix

| | | |
|---|---|---|
| 5′ AgeI huVK1-5 | CTTGTCCTTGTTTTAACCGGTGTCCAGTGTGACATCCA | 74 |
| | GATGACCCAGTC | |
| 5′ AgeI huVK1-9 | CTTGTCCTTGTTTTAACCGGTGTCCAGTGTGACATCCA | 75 |
| | GTTGACCCAGTCT | |
| 5′ AgeI huVK1-8/ | CTTGTCCTTGTTTTAACCGGTGTCCAGTGTGYCATCYG | 76 |
| 1D-8 | GATG | |
| 5′ AgeI huVKID-43 | CTTGTCCTTGTTTTAACCGGTGTCCAGTGTGCCATCCG | 77 |
| | GATGACCCAGTC | |
| 5′ AgeI huVK2-24 | CTTGTCCTTGTTTTAACCGGTGTCCAGTGTGATATTGT | 78 |
| | GATGACCCAGAC | |
| 5′ AgeI huVK2-28 | CTTGTCCTTGTTTTAACCGGTGTCCAGTGTGATATTGT | 79 |
| | GATGACTCAGTC | |
| 5′ AgeI huVK2-30 | CTTGTCCTTGTTTTAACCGGTGTCCAGTGTGATGTTGT | 80 |
| | GATGACTCAGTC | |
| 5′ AgeI huVK3-11 | CTTGTCCTTGTTTTAACCGGTGTCCAGTGTGARATYGT | 81 |
| | GYTGACMCAGWC | |
| 5′ AgeI huVK3-11/ | CTTGTCCTTGTTTTAACCGGTGTCCAGTGTGAAATWGT | 82 |
| 3-15 | GWTGAYSCAG | |
| 5′ AgeI huVK3-15 | CTTGTCCTTGTTTTAACCGGTGTCCAGTGTGAAATAGT | 83 |
| | GATGACGCAGTC | |
| 5′ AgeI huVK3D-15 | CTTGTCCTTGTTTTAACCGGTGTCCAGTGTGAAATAGT | 84 |
| | GATGAYGCAG | |
| 5′ AgeI huVK3-20 | CTTGTCCTTGTTTTAACCGGTGTCCAGTGTGAAATTGT | 85 |
| | GTTGACRCAGTCT | |
| 5′ AgeI huVK4-1 | CTTGTCCTTGTTTTAACCGGTGTCCAGTGTGACATCGT | 86 |
| | GATGACCCAGTC | |

Human Kappa Light Chain: 3′-BsiWI huVK Reverse Primer mix

| | | |
|---|---|---|
| 3′ BsiWI huJK 1/4 | GATGGCGCCGCCACCGTACGYTTGATYTCSACCTTKGT | 87 |
| 3′ BsiWI huJK 1/4B | GATGGCGCCGCCACCGTACGYTTKAAYTCSACCTTKGT | 88 |
| 3′ BsiWI huJK 2E | GATGGCGCCGCCACCGTACGYGTGATYTCSACCTTKGT | 89 |
| 3′ BsiWI huJK 2 | GATGGCGCCGCCACCGTACGYTTGATYTCCAGCTTKGT | 90 |
| 3′ BsiWI huJK 2B | GATGGCGCCGCCACCGTACGYTTGATCCCCAGCTTKGT | 91 |
| 3′ BsiWI huJK 2C | GATGGCGCCGCCACCGTACGNGTGATYTCCAGCTTKGT | 92 |
| 3′ BsiWI huJK 2D | GATGGCGCCGCCACCGTACGYTTGATCTCCCAGCTTGG | 93 |
| 3′ BsiWI huJK 2-28 | GATGGCGCCGCCACCGTACGNKTGATYTCNACGCTTGG | 94 |
| 3′ BsiWI huJK 3 | GATGGCGCCGCCACCGTACGTTTGATATCCACTTTGGT | 95 |
| | C | |
| 3′ BsiWI huJK 4 | GATGGCGCCGCCACCGTACGYCTGATYTCNACCTTKGT | 96 |
| 3′ BsiWI huJK 5 | GATGGCGCCGCCACCGTACGYTTAATCTCCAGTCGTGT | 97 |
| | C | |

*The primer mixes for the second-round PCRs included 20-30 bp sequences complementary to the 5′- or 3′- ends of the heavy- or kappa light chain expression vector. Sequences targeting the restriction enzyme sites are highlighted in red while the sequences in blue are part of the vector backbone.

The amplified IGHV and IGκV fragments were assembled in vitro using the SLIC method with the in-house generated pVCHIg-hG1 and pVCLIg-hK expression vectors, respectively. Briefly, 50 ng of the IGHV and IGκV fragments were independently combined with 100 ng of the respective linearized expression vectors, assembled in vitro as part of a SLIC reaction, and then transformed into chemically competent E. coli cells. Standard transformation was performed followed by spread-plating onto Luria-Bertani (LB)-agar Petri dishes containing 100 μg/mL Kanamycin. Screening of single bacterial colonies containing the IGHV/IGκV inserts was also performed by PCR, and positively screened cultures were grown for 16-18 h at 37° C. with moderate shaking in 5 mL LB (Difco Laboratories) broth containing 100 μg/mL Kanamycin (Sigma-Aldrich Inc., St. Louis, MO, USA). Plasmid DNA was purified with QIAprep Spin columns (Qiagen, Santa Clarita, CA, USA) and sent for sequencing to confirm the insert identity with the original PCR products.

### Human Ig Gamma Constant Region Identification

To determine the natural Ig isotype of the ZIKV-CR huMAbs from donor KH1891, another PCR was set up to amplify the human Ig gamma (γ) constant fragment or Fc region was amplified from freshly prepared cDNAs of the original ZIKV-CR B cells. Universal oligonucleotide primers were designed to target the Fc region spanning domains 1-3 (γC_{H}1-3) and the hinge region (Table 4). Internal primers targeting γC_{H}2 were also designed for use as sequencing primers. All PCR reactions were performed with a total volume of 50 μL per sample containing 0.4 μM each of the forward or reverse primers, 400 μM each of the dNTP mix, and 1.25 U One Taq DNA Polymerase (Invitrogen, Carlsbad, CA, USA). Each round of PCR consisted of initial denaturation for 60 s at 94° C., then 45 cycles of denaturation at 94° C. for 30 s, annealing at 65° C. for 30 s, and extension at 68° C. for 60 s, followed by a final extension of 68° C. for 5 min and a cool down to 4° C. Successfully amplified Ig Fc genes, including controls, were visualized by gel electrophoresis on a 2% (wt/vol) agarose gel and sent for sequencing to confirm the identity.

TABLE 4
Universal primers used for the amplification
of human Ig gamma constant domains

| Target | Primer | Primer sequence | SEQ ID |
|---|---|---|---|
| Fragment | Name | (5′ ---> 3′) | NOs |
| Human Ig | [symbol] CH1 FP | 5′ --- GCYWSCACCA | 98 |
| Gamma | | AGGGCCCATCGGTCTTC | |
| CH1-CH3 | | CC --- 3′ | |
| | [symbol] CH3 RP | 5′ --- TCATTTACCC | 99 |
| | | GGAGACAGGGAGAGGCT | |
| | | CTTCTG --- 3′ | |
| Human Ig | [symbol] CH2 mid | 5′ --- TGAACTCCTG | 100 |
| Gamma | FP | GGAGGACCGTCAGTCTT | |
| CH2 | | CCTCTTCC --- 3′ | |
| (middle) | | | |
| | [symbol] CH2 mid | 5′ --- GGAAGACTGA | 101 |
| | RP | CGGTCCTCCCAGGAGTT | |
| | | CAGGTGC --- 3′ | |

### Human Ig Gene Sequence Analysis

To define the Ig gene structure, particularly the framework, V(D)J gene assignment, and the complementarity determining region (CDR) boundaries, the inventors analyzed the sequences by comparing the variable gene of the generated IGHV and IGκV fragments with the human Ig sequences using the international ImmunoGenetics (IMGT)/V-Quest germline gene database. The closest V(D)J or V-J genes and alleles for the respective heavy and kappa light chains were identified by alignment of the first nucleotide to the conserved second cysteine codon of the V-region in the germline database. Only the sequences with at least 70% V-gene and allele identity with the germline were expressed. Following the IMGT numbering delimitations, analysis of the junction region showed the translated Ig CDR3, which extends from the conserved cysteine (Cys104) to the conserved tryptophan-glycine (Trp-Gly-X-Gly) motif in all human JH gene segments or the conserved phenylalanine-glycine motif (Phe-Gly-X-Gly) in all human Jr gene segments. Non-silent nucleotide mutations at the V-regions were also generated, which gave rise to distinct amino acid changes. All B cell clones with productive V-genes were expressed. Sequences that were non-productive, out-of-frame, or with premature stop codons were excluded from expression. Sequences matching light or heavy chain sequences from a pseudogene in the germline were also omitted. All the remaining paired sequences with accurate and productive antibody transcripts were expressed as full-length human IgG1 in this study.

Somatic hypermutation (SHM) level (% SHM) was calculated as the divergence of the antibody variable domains from the assigned closest germline sequences at the nucleotide level. Similarly, the amino acid (AA) changes resulting from non-silent nucleotide mutations (% AA change) were evaluated as the divergence in the amino acids relative to the number of germline AA. AA changes were identified following the IMGT physicochemical classifications based on hydropathy, volume, and IMGT physicochemical properties, and whether the AA change belongs to the same or different class as indicated by (+) or (−), respectively. IMGT initially established four types of AA changes: very similar (+++), similar (++−, +−+), dissimilar (−−+, −+−, +−−), and very dissimilar (−−−). The inventors simplified this grouping into two categories: similar or dissimilar AA changes, wherein similar AA changes follow all (+++) or (++−, +−+) any two of the IMGT similarity criteria, while dissimilar AAs belong to only one (−−+, −+−, +−−) or completely different (−−−) IMGT classification.

### Antibody Expression, IgG Quantification, and Purification

Adherent human embryonic kidney (HEK) 293T cells were transfected with equimolar amounts of in-house generated human immunoglobulin G1 (IgG1) heavy and kappa light chain expression vectors using branched polyethylenimine (PEI) (Sigma-Aldrich Inc., St. Louis, MO, USA) at an optimal 1:3 total DNA/PEI ratio. Transient expression of recombinant human IgG antibodies was performed in micro-scale and large-scale cultures for 5 and 7 days, respectively. The collected cell culture supernatants containing the secreted antibodies were clarified by centrifugation at 3000×g for 10 min, neutralized with phosphate-buffered saline to a final concentration of 1×, passed through 0.20 μm filters (Sartorius, Gottingen, DE), and analyzed for MAb concentration by indirect ELISA using purified recombinant Protein A and Protein G fusion proteins immobilized onto 96-well plates. For large-scale expression, human IgG1 or IgG3 monoclonal antibodies were purified by affinity chromatography using Protein G-coupled agarose beads (Millipore, MA, USA). After binding the supernatant with the beads for 16-18 h at 4° C., the beads were manually packed onto a 1 mL-polypropylene chromatography column (Qiagen, Santa Clarita, CA, USA) at a flow rate of 1 mL/min, followed by equilibration with 10 column volumes (CV) of PBS (pH 7.0) washing buffer. The antibody was eluted with 0.1 M Glycine buffer (pH 2.3) onto 96-well plates containing 50 μL 1 M Tris-HCl (pH 8.9) in twenty-four successive fractions of 300 μL. High-antibody fractions were collected per antibody, and each was dialyzed against Tris buffer (pH 6.5) thrice at 4° C. using Slide-A-Lyzer® dialysis cassettes (Thermo Fisher Scientific Inc., Rockford, IL, USA). The purified IgG concentration was estimated using a Qubit® protein assay kit (Life Technologies, Thermo Fisher Scientific Inc., Rockford, IL, USA). For F(ab) and F(ab′)₂ antibody production, the transformed cells were utilized to grow and express the histidine-tagged K8b-F(ab) and K8b-F(ab′)₂ antibodies, and the proteins were purified using Ni-NDA affinity chromatography according to the manufacturer's protocol (Chelating Sepharose Fast Flow; GE Healthcare, Uppsala, SWE).

The purity of the full-length K8b-IgG1, K8b-IgG3, K8b-F(ab′)₂, and K8b-F(ab) antibody preparations was assessed by sodium dodecyl sulfate-polyacrylamide gel electrophoresis (SDS-PAGE). Specifically, purified proteins (1 μg) were mixed with a nonreducing Laemmli sample buffer, resolved on a 4-20% gradient polyacrylamide gel (Mini-Protean TGX; Bio-Rad, Hercules, CA, USA) in Tris-glycine SDS running buffer, and stained with Coomassie blue dye.

### Immunoglobulin G Enzyme-Linked Immunosorbent Assay (IgG ELISA)

An IgG ELISA was used to test the presence and binding reactivity of antigen-specific IgGs among the dengue-negative or healthy and dengue-infected polyclonal sera or in the cell-culture supernatants co-cultivated with B cells or of the purified human monoclonal antibodies using a similar antigen-capture ELISA protocol with minor modifications. Briefly, flat-bottom 96-well MaxiSorp NUNC-Immuno plates (NUNC, Roskilde, DK) were coated with 50 μL of serotype-specific rabbit anti-DENV virus-like particles (VLP) sera (1:500), rabbit anti-JEV VLP sera (1:3,200) or rabbit anti-ZIKV VLP (1:8,000) sera at indicated dilutions in bicarbonate buffer (0.015 M Na₂CO₃, 0.035 NaHCO₃, pH 9.6), incubated overnight at 4° C., and blocked with 200 μL of 5% milk in 1×PBS with 0.1% Tween-20 (5% milk/PBST) for 1 h at 37° C. Equimolar amounts of DENV, JEV, or ZIKV VLPs diluted in the blocking buffer were added to each well, incubated for 2 h at 37° C., and washed five times with 200 μL of 0.1% PBST. The transient expression of each VLP in this study was carried out as described using previously established transcriptionally and translationally optimized eukaryotic cell expression plasmids. The amounts and concentrations of the actual VLPs used were equalized in all wells by dilution to ˜25 ng VLP per well, as the inventors have previously determined. The VLP concentration was determined from the standard curves generated using purified antigens following a sigmoidal dose-response analysis using GraphPad Prism (version 9.5.1, GraphPad Software, Inc., La Jolla, CA, USA). Individual human or mouse polyclonal sera were initially diluted at 1:1,000, titrated two-fold, added into wells in duplicate, and incubated for 1 h at 37° C. Dengue-negative or healthy controls, pre-vaccination mouse sera, untransfected HEK293T cell culture supernatant, or IgG isotype control (in-house generated SARS-CoV-2 virus-specific huMAb) were used as negative antibody controls for ELISAs. A similar ELISA protocol was used to determine the binding reactivity of isolated human or murine monoclonal antibodies, initially diluted to 10 μg/mL, followed by two-fold titration. Incubations with the donkey anti-human IgG (1:5,000, Sigma-Aldrich, St. Louis, MO, USA) or goat anti-mouse IgG (1:5,000, Sigma-Aldrich, St. Louis, MO, USA) conjugates and substrate were carried out according to the standard ELISA. The OD450 values, modeled as nonlinear functions of the log 10 serum dilutions using a sigmoidal dose-response (variable slope) equation and endpoint antibody titers from two independent experiments, were determined as the dilutions where the OD value was twice the average OD of the negative control.

As for antigen-specific human IgG and IgG3 detection, the in-house developed antigen-capture ELISA was performed using serum-free grown ZIKV virus-like particles (VLPs) as capture antigens described previously with minor modifications. Polyclonal dengue-infected and healthy control plasma were added to the precoated plates and incubated for 1 h at 37° C. VLP-reactive total human IgG or IgG3 were detected using HRP-conjugated donkey anti-human IgG or mouse anti-human IgG3 (Invitrogen, Thermo Fisher Scientific, Inc., Rockford, IL, USA) for 1 h at 37° C. at 1:5,000 or 1:1,000, respectively.

The binding of purified K8b-F(ab) and K8b-F(ab′)₂ molecules was determined alongside purified K8b-IgG1 using the VLP capture ELISA described above. Bound F(ab) and F(ab′)₂ molecules to the VLPs were detected with 1:40,000 HRP-conjugated goat anti-human F(ab) (Jackson ImmunoResearch Laboratories Inc., West Grove, PA, USA). After washing with PBS ten times, TMB substrate was added into the wells, incubated for 10 min, and the reaction was stopped with 2N H₂SO₄. Reactions were measured at A_{450/630}. The relative binding reactivity of F(ab) and F(ab′)₂ molecules was determined by comparing the P/N values generated from the replicate wells with that of IgG1 molecules.

K8b-IgG1 and mD2VLP Immune Complex Formation and Cryo-EM 3D Reconstruction

The previously developed and characterized mature dengue 2 VLPs (mD2VLPs) were generated by transfecting HEK293T cells with the recombinant pVD2i-C18S plasmid using Lipofectamine® 2000 DNA Transfection Reagent (cat. 11668019, Thermo Fisher Scientific, USA) according to the manufacturer's instructions. The culture supernatants were harvested and purified by sucrose gradient centrifugation in TNE buffer (50 mM Tris-HCl, 100 mM NaCl, 0.1 mM EDTA) using a Beckman SW-41 rotor. The fraction with the highest OD reading in ELISA was collected, and the VLPs were resuspended in TNE buffer. These samples were further concentrated using Amicon® Ultra-0.5 centrifugal filter units with 100 kDa cut-off. To form the immune complexes of K8b-IgG1 and mD2VLP, K8b-IgG1 was mixed with purified and concentrated mD2VLPs at a molar ratio of 30 molecules of K8b-IgG1 per mD2VLP particle to prevent inter-spike crosslinking. The complex was incubated at 4° C. overnight. The fresh immune complexes were immediately applied to a glow-discharged Quatifoil 2/2 grid (Quatifoil GmbH, DE) for cryo-EM grid preparation. After removing the excess liquid, the grid was rapidly frozen in liquid nitrogen-cooled liquid ethane using a Gatan CP3. Cryo-EM images were captured at 15,000× magnification with an accelerating voltage of 200 kV using a JEM-2100F transmission electron microscope and a direct electron detector (DE-12 Camera System-Direct Electron, LP) with 6.0 μm pixel pitch (˜4 Å at the specimen level). The image processing and 3D reconstruction were performed by a single-particle approach using the EMAN2 software package. Accordingly, 2,035 spiky and spherical particles were manually selected. Contrast transfer function parameters were estimated internally based on the boxed particles (e2ctf.py). Then, 2D reference-free averaging was performed using the default parameters in EMAN2 and generated an initial de novo model based on the 2D class averages, with icosahedral symmetry imposition. All further refinements were performed with automasking filtering using EMAN2. The final resolution of the structure, incorporating an unresolved Fc fragment, was determined to be 16.3 Å, based on the gold-standard Fourier shell correlation (FSC) at 0.143. To elucidate the conjoining of the F(ab) fragments, a refinement protocol employing a more permissive masking strategy was implemented, which yielded a structural configuration characterized by the discernible presence of the Fc region juxtaposed with F(ab) fragments exhibiting suboptimal resolution.

### Annotation of Antibody CDRs and Antibody Modeling Using AlphaFold2 and MODELLER

The three-dimensional structure of K8b-IgG1 was determined using the AlphaFold tool in UCSF ChimeraX (version 1.3). The antibody was modeled independently from the antigens using the complete AlphaFold2 pipeline with default parameters. The variable heavy and light chain sequences of K8b have been concatenated. Antibody complementarity-determining regions (CDRs) were annotated with the AHo numbering scheme by the ANARCI program. To evaluate the modeling accuracy of the CDRs, the inventors first superimposed the ±4 flanking amino acid residues of each CDR in predicted models onto native antibody structures. Next, the RMSD of the CDRs was calculated by Prody 2.0. The paratope RMSD was computed similarly after superimposing the paratope of the model onto the native structure.

Simultaneously, the three-dimensional structure of K8b-IgG1 was predicted by comparative modeling using MODELLER (v. 4.0). The crystal structure of HuMAb P5A-2G9 (PDB ID: 7CZT) was chosen to model the F(ab) domain and the crystal structure of mouse MAb IgG2a (PDB ID: 1IGT) to model the Fc domain and the glycan chains. E proteins and K8b-IgG1 F(ab) domains were fitted rigidly using the Fit-in-Map tool in UCSF Chimera with the maximized CCC (Cross-Correlation Coefficient) score. The symmetry restraints were applied to avoid clashes between neighboring protein molecules. Due to its symmetrical nature, the F(ab) domain would have two-fold rotation-related binding poses, which cannot be distinguished under a low-resolution map. To overcome this ambiguity, the inventors used the protein docking software HEX to calculate the binding energy and determine the most probable binding orientation of the F(ab) domain with another molecule. To further refine the immune complex structure and optimize the antigen-antibody interface, the inventors conducted the flexible fitting procedure using the Flex-EM module in MODELLER. The secondary structure elements in E protein and F(ab) were treated as rigid bodies during the fitting. To reduce the computation expense, only half the size of the F(ab) and three E dimers were included in the molecular dynamics simulation. The inventors performed 20 independent simulated annealing molecular dynamics runs and picked ten final conformers with lower binding free energies for subsequent analysis. The antigen-antibody interfaces were analyzed using PDBePISA and PDBsum. The structure figures in this article were created using Chimera or PyMOL (v. 2.5.4).

### SDM and Epitope Mapping Experiments

To validate the predicted binding site of K8b-IgG1 on mD2VLP, the inventors used the ZIKV VLP by focusing on the amino acid residues that are: (1) conserved among ZIKV, JEV, and the four serotypes of DENV based on sequence alignment; (2) located on loops with enhanced solvent accessibility based on SASA results; and (3) candidate epitope from previously reported broadly neutralizing antibodies. The previously published and transcriptionally optimized eukaryotic cell expression plasmid transiently expressing the envelope (prM/E) proteins from ZIKV was used in this study. Three amino acid residues in the be loop region of the ZIKV envelope (E) protein, namely arginine (R73), threonine (T76), and aspartic acid (D87), were separately mutated to alanine (A). Single-point mutations were also introduced in other loops that span the ZIKV envelope protein, such as the fusion loop (FL) (N103), fg loop (R193), hi loop (T231), IoA loop or ED I/III linker (G302), and the EDIII DE loop (T366). The inventors also incorporated residues 101 and 103 at the fusion loop for site-directed mutagenesis (SDM) since the binding footprint of the EDE antibodies centered on residue 101 of E protein.

The mutations in the ZIKV E were generated by overlapping PCR with the appropriate pairs of complementary forward (F) and reverse (R) primers (Table 5) according to the manufacturer (QuikChange™ II site-directed mutagenesis kit, Stratagene, La Jolla, CA, USA). Briefly, the PCR reaction of 50 μL contained 200 ng of template, 0.5 μM primer pair, 200 μM dNTPs, and 1 Unit of Pfu Ultra II Fusion HS DNA Polymerase. The PCR cycles were initiated at 95° C. for 2 min to denature the template DNA, followed by 18 cycle amplification cycles, with each cycle consisting of 95° C. for 30s, T_{m no}−5° C. for 30s, and 72° C. for 8 min. The PCR cycles were finished with an extension step at 72° C. for 8 min. All PCR products were treated with 5 Units of DpnI at 37° C. for 2 h, followed by visualization on agarose gel electrophoresis. An aliquot of the PCR products was transformed into E. coli competent cells using the standard transformation protocol. The transformed cells were spread on a Luria-Bertani (LB) plate containing 50 μg/mL of Ampicillin (Cyrusbioscience Inc., New Taipei, TW), incubated overnight at 37° C., and isolated for plasmid DNA. All desired mutations were confirmed by DNA Sanger sequencing. The resulting pCDNA3-ZIKV plasmids expressing the full-length envelope protein with single-site mutations, R73A, T76A, D87A, N103A, R193A, T231A, G302A, and T366A were named after the mutation. Following transient expression on HEK293T, the wild-type and mutant ZIKV VLP-expressing cell culture supernatant were collected, titrated for concentrations, and used for binding IgG-ELISAs described above. Most mutant ZIKV VLPs were expressed at 60-100% efficiency except for ZIKV VLP N103A, which only showed 2% VLP expression, indicative of a disrupted VLP formation; hence, its exclusion in binding ELISA and further analyses.

TABLE 5
Primers used for site-directed mutagenesis on ZIKV VLP

| | | | | SEQ | | Mis- | |
|---|---|---|---|---|---|---|---|
| Original | AA | Primer | Primer sequence | ID | Length, | matches, | Tm |
| AA | position | name | (5′ ---> 3′) | NO | bp | bp | (° C.) |
| Arginine, | 73 | R73A | 5′ --- | 102 | 33 | 2 | 69 |
| R | | FP | ATGGCCAGCGACAGCgcATGCC | | | | |
| | | | CCACACAGGGC --- 3′ | | | | |
| | | R73A | 5′ --- | 103 | 33 | 2 | 69 |
| | | RP | GCCCTGTGTGGGGCATgcGCTG | | | | |
| | | | TCGCTGGCCAT --- 3′ | | | | |
| Threonine, | 76 | T76A | 5′ --- | 104 | 32 | 1 | 71 |
| T | | FP | ACAGCAGATGCCCCgCACAGGG | | | | |
| | | | CGAGGCTTAC --- 3′ | | | | |
| | | T76A | 5′ --- | 105 | 32 | 1 | 68 |
| | | RP | GTAAGCCTCGCCCTGTGcGGGG | | | | |
| | | | CATCTGCTGT --- 3′ | | | | |
| Aspartic | 87 | D87A | 5′ --- | 106 | 33 | 1 | 67 |
| Acid, D | | FP | CTGGACAAGCAGTCCGcCACCC | | | | |
| | | | AGTACGTGTGC --- 3′ | | | | |
| | | D87A | 5′ --- | 107 | 33 | 1 | 70 |
| | | RP | GCACACGTACTGGGTGgCGGAC | | | | |
| | | | TGCTTGTCCAG --- 3′ | | | | |
| Asparagine, | 103 | N103A | 5′ --- | 108 | 31 | 2 | 67 |
| N | | FP | GGATAGAGGCTGGGGCgcTGGC | | | | |
| | | | TGCGGCCTG --- 3′ | | | | |
| | | N103A | 5′ --- | 109 | 31 | 2 | 67 |
| | | RP | CAGGCCGCAGCCAgcGCCCCAG | | | | |
| | | | CCTCTATCC --- 3′ | | | | |
| Arginine, | 193 | R193A | 5′ --- | 110 | 33 | 2 | 64 |
| R | | FP | CTGGACTGCGAGCCTgcAACCG | | | | |
| | | | GCCTGGATTTC --- 3′ | | | | |
| | | R193A | 5′ --- | 111 | 33 | 2 | 64 |
| | | RP | GAAATCCAGGCCGGTTgcAGGC | | | | |
| | | | TCGCAGTCCAG --- 3′ | | | | |
| Threonine, | 231 | T231A | 5′ --- | 112 | 36 | 1 | 75 |
| T | | FP | TGGCATGCCGGCGCTGATgCAG | | | | |
| | | | GCACACCCCACTGG --- 3′ | | | | |
| | | T231A | 5′ --- | 113 | 36 | 1 | 75 |
| | | RP | CCAGTGGGGTGTGCCTGcATCA | | | | |
| | | | GCGCCGGCATGCCAG --- 3′ | | | | |
| Glycine, | 302 | G302A | 5′ --- | 114 | 34 | 1 | 69 |
| G | | FP | CAAGCTGCGGCTGAAGGCCGTG | | | | |
| | | | TCCTACAGCCTG --- 3′ | | | | |
| | | G302A | 5′ --- | 115 | 34 | 1 | 72 |
| | | RP | CAGGCTGTAGGACACGgCCTTC | | | | |
| | | | AGCCGCAGCTTG --- 3′ | | | | |
| Threonine, | 366 | T366A | 5′ --- | 116 | 35 | 1 | 71 |
| T | | FP | CCGCCAACCCTGTGATCgCCGA | | | | |
| | | | GAGCACCGAGAAC --- 3′ | | | | |
| | | T366A | 5′ --- | 117 | 35 | 1 | 69 |
| | | RP | GTTCTCGGTGCTCTCGGcGATC | | | | |
| | | | ACAGGGTTGGCGG --- 3′ | | | | |

For all primers, the mutagenesis oligonucleotides were denoted in lower case and colored in red.
Tm is the melting temperature of mutagenic oligonucleotide primers annealing with the WT template.
AA = amino acids;
bp = base pairs.

### Solvent Accessibility Estimation of E Protein in the VLP Structure

EasyModeller (v. 4.0) was used to align the E protein sequences from DENV-1, DENV-3, DENV-4, ZIKV, and JEV onto the DENV-2 E protein structure in the previously published mD2VLP structure. After generating the monomeric E protein structure using MODELLER, an in-house generated PyMOL script was used to transfer the T=1 symmetry of mD2VLP to the E protein, and to generate the VLP structures of the five viruses. The derived VLP structures were subjected to calculate the solvent accessibility (% SASA, the fractional solvent-accessible surface area of the amino acid residue) using POPS.

### Sequence Analysis of Flavivirus E Proteins

Flavivirus E protein sequence alignments were performed using Clustal Omega software with the representative prototype strains and the GenBank accession numbers as follows: DENV-1 Hawaii (KM204119), DENV-2 16681 (KU725663), DENV-3 (KU050695), DENV-4 BC 71/94 (MW945661), JEV CJN-S1 (AY303793), ZIKV PRVABC59 (MK713748), and ZIKV MR766 (NC_012532).

### Mouse Experiments

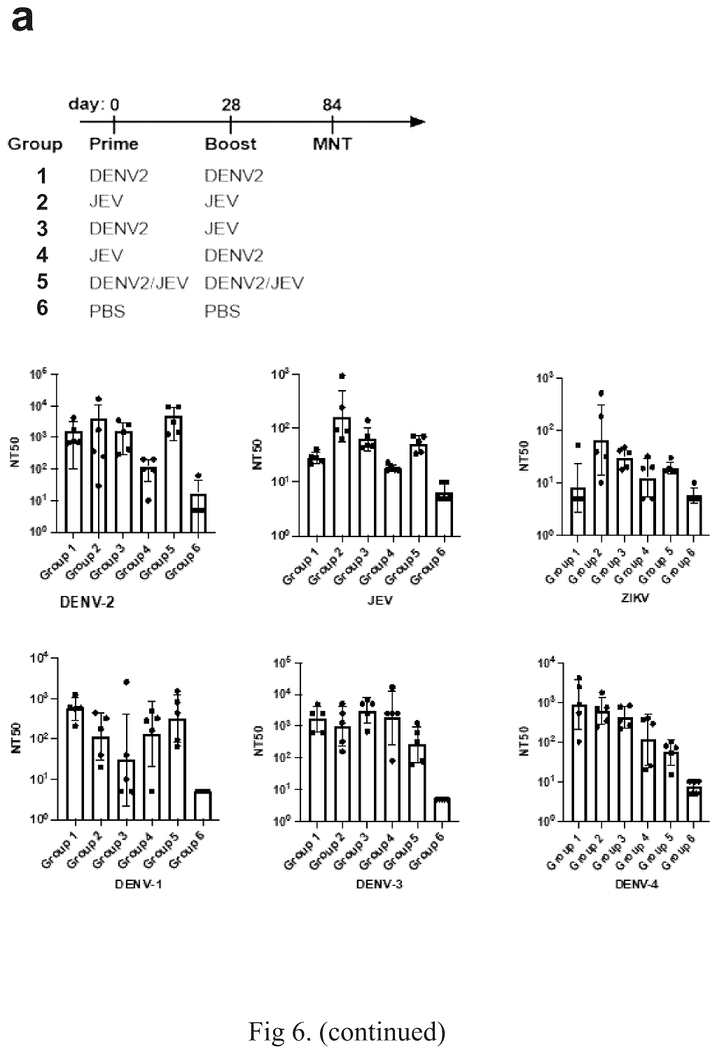

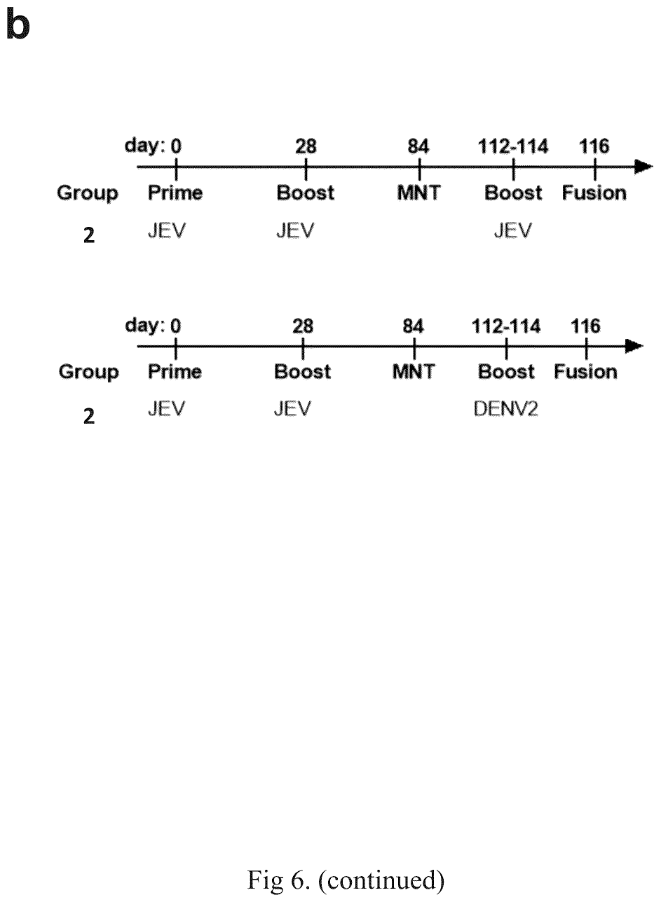

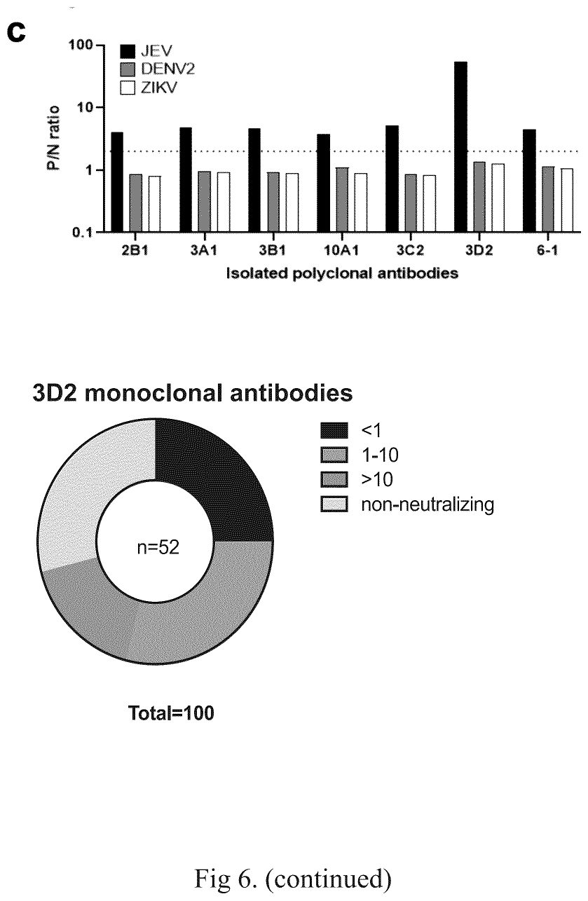

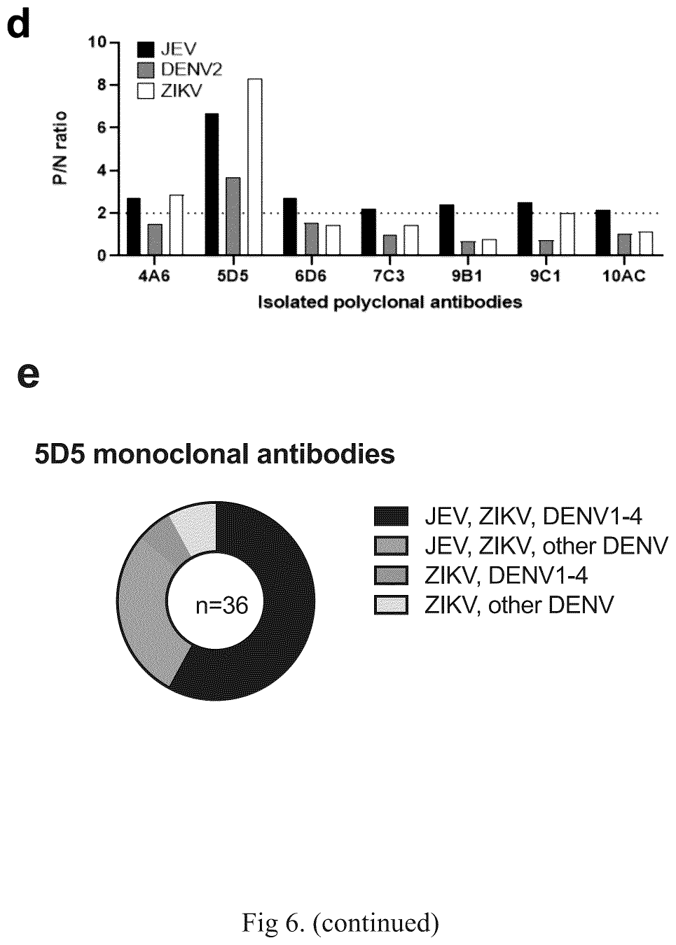

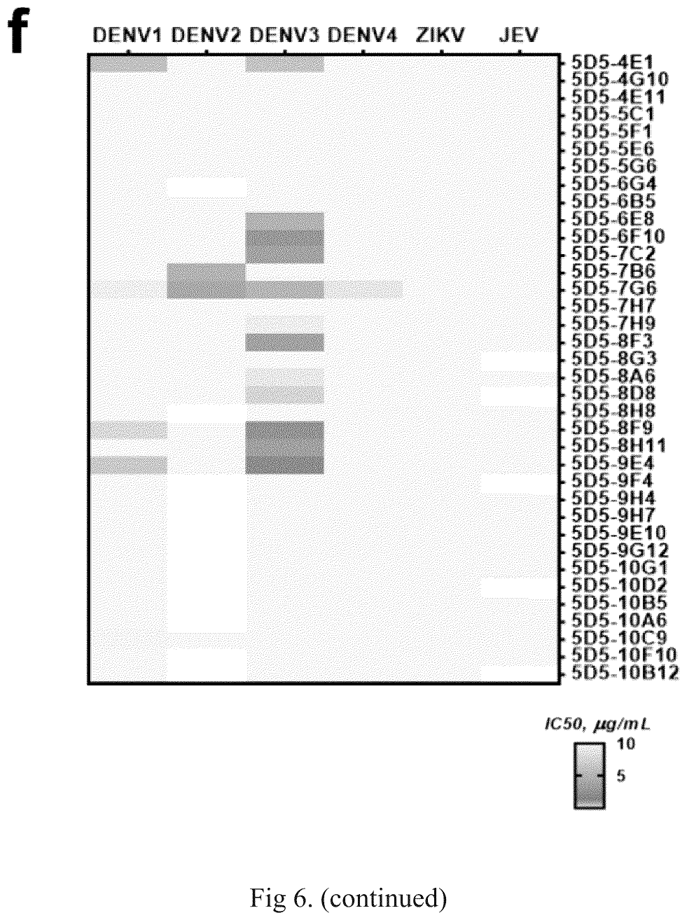

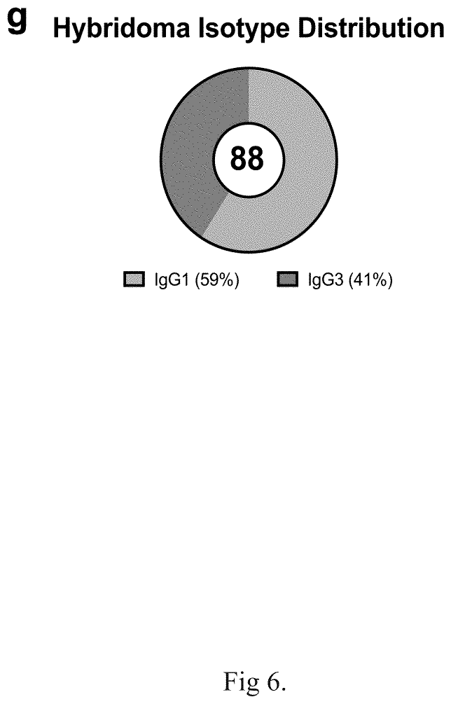

BALB/c mice were used in all experiments. This study was carried out in compliance with the guidelines for the care and use of laboratory animals of the National Laboratory Animal Center, Taiwan. All animal-use and care procedures were reviewed and approved by the Institutional Animal Care and Use Committee (IACUC) of the National Chung Hsing University (Approval Number: 106-101R2). All efforts were made to minimize the suffering of the mice. The immunization schedule using different VLP prime-boost strategies is shown in FIG. 6a. Groups of five 3-week-old mice were immunized intramuscularly with 4 μg of each purified JEV VLP and/or mD2VLP in 100 μL of 1×PBS (50 μL in each thigh) at 0 and 28 days. VLPs used in immunizing the mice were not adjuvanted. Mice were bled retro-orbitally at 84 days post-vaccination, and individual serum specimens were evaluated for flavivirus neutralization activity as described above.

### Hybridoma Generation and Murine mAb Screening

Hybridomas secreting anti-flavivirus monoclonal antibodies were generated from the most responsive JEV VLP-vaccinated mice (JEV FRμNT50>120) following the standard protocol with minor modifications. Briefly, JEV VLP-vaccinated mice were boosted intraperitoneally with either 4 μg of purified JEV VLP or mD2VLP emulsified in Freund's incomplete adjuvant (Sigma-Aldrich, St. Louis, MO, USA) for three consecutive days. Two days after the last boost, the splenocytes were harvested and then individually fused with NSI/1-Ag4-1 myeloma cells using 50% polyethylene glycol (PEG) (PEG Hybri-Max™ Sigma-Aldrich, St. Louis, MO, USA). Fused cells were resuspended in RPMI supplemented with 20% FBS, hypoxanthine-aminopterin-thymidine medium, and hybridoma growth factor (Nutridoma™-CS, Roche Diagnostics, Indianapolis, IN, USA). Two weeks post-fusion, supernatants from the selected polyclonal colonies were individually screened for secretion of mAbs by IgG ELISA as described above using JEV, DENV-2, and ZIKV VLPs, and detected with an HRP-conjugated goat anti-mouse IgG. The positive clones (P/N values of ≥2) were subcloned by limiting dilution to isolate monoclonal cells and then further screened for VLP ELISA binding and flavivirus neutralization activities using unpurified supernatants.

### Murine Monoclonal Antibody Isotyping

Cell culture supernatant containing antibody-secreting hybridomas were analyzed for class and subclass identity, specifically murine IgG1, IgG2a, IgG2b, IgG3, IgA, and IgM, as well as light-chain identity (Kappa or lambda light chains), following the manufacturer's protocol (Pierce® Rapid Mouse Antibody Isotyping Kit, Thermo Fisher Scientific Inc., Rockford, IL, USA). Briefly, the culture supernatant was diluted to a final concentration of 1:100 using Tris-buffered saline (20 mM Tris; 150 mM NaCl). Fifty microliters (50 μL) of the diluted antibody mixture and 50 μL of HRP-conjugated goat anti-mouse IgG⁺IgA⁺IgM antibodies were simultaneously added to each of the ELISA strip-wells precoated with six different anti-mouse heavy-chain or two light-chain capture antibodies. The samples were mixed by gently tapping the plate, incubated for 1 h at room temperature, and washed with the proprietary wash buffer. Finally, adding TMB substrate followed by 2N H₂SO₄ stop solution revealed the antibody isotype based on which wells in the strip produced color. Wells with the highest response (or darkest color) based on visual inspection or spectrophotometer readout at 450 nm/630 nm indicate isotype and light chain compositions.

### Statistics and Reproducibility

All data were represented as means±standard deviations (SD) and were analyzed using GraphPad Prism (version 9.5.1, GraphPad Software Inc.). Statistical significance was determined using a one-way analysis of variance (ANOVA) followed by Tukey's multiple comparisons post-test to analyze the ELISA and neutralization titers across multiple groups. The correlation between IgG3 and neutralization titers was calculated using Pearson's correlation and the Wilcoxon matched-pairs signed rank test. P values less than 0.05 were considered significant.

### Results

High ZIKV Neutralization Titers Among Dengue-Infected Individuals with Pre-Existing JEV Immunity

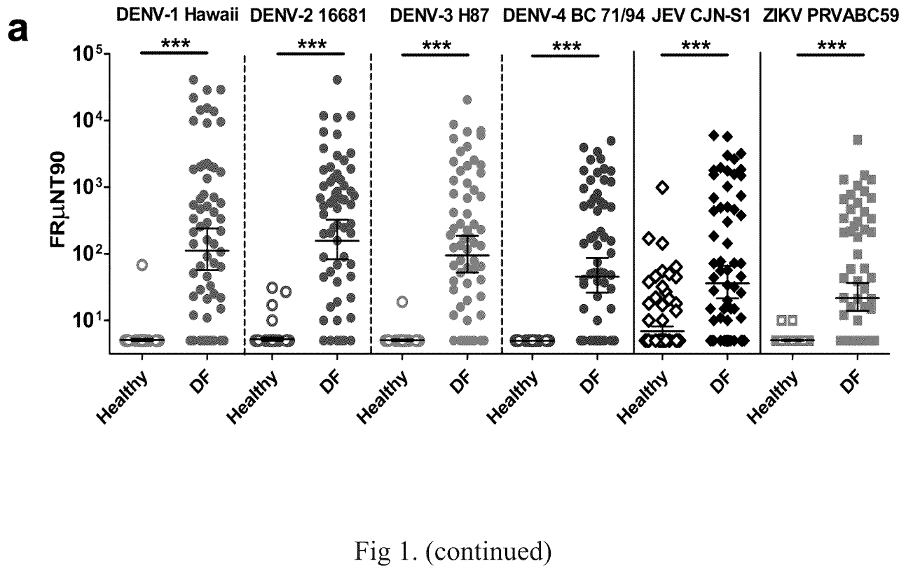

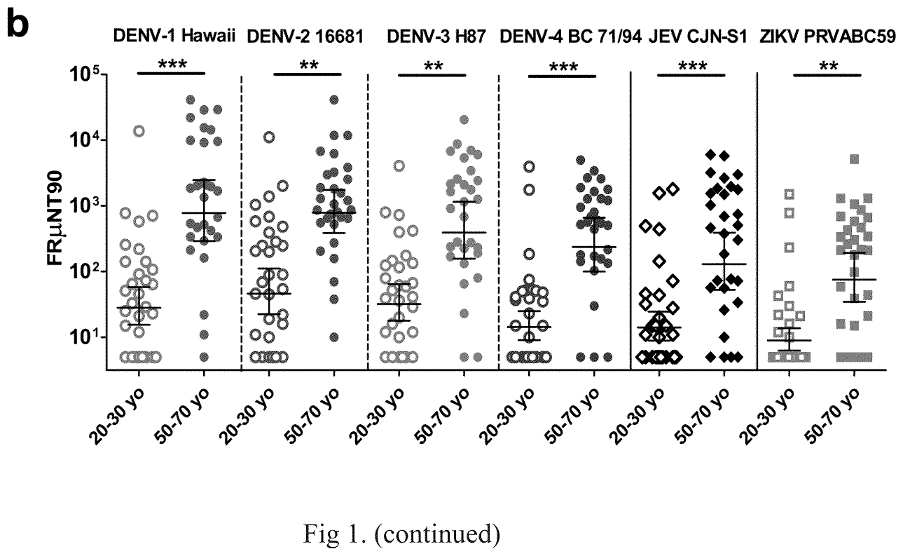

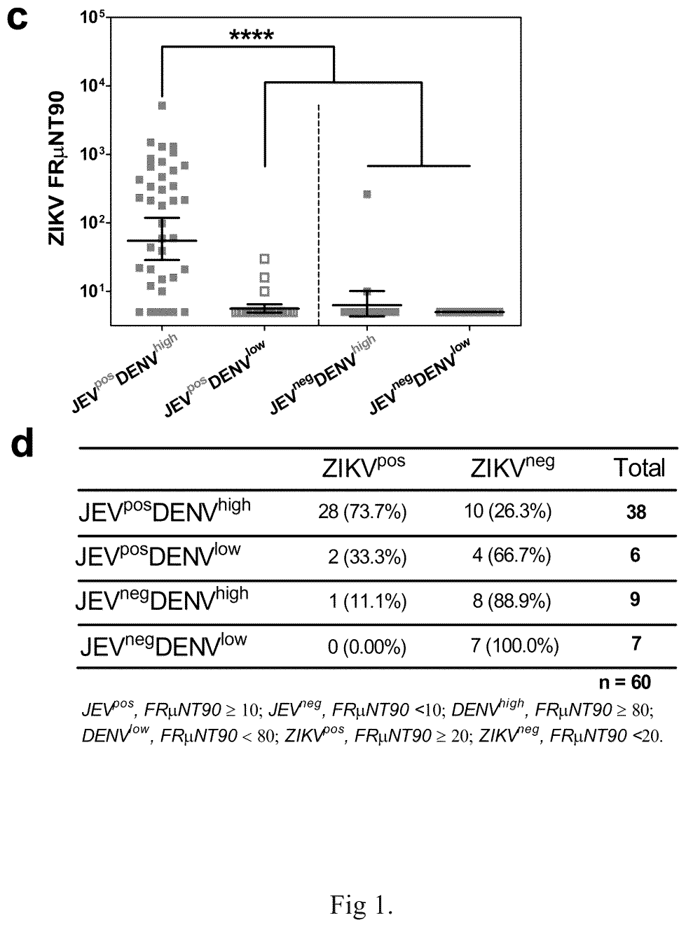

Based on the nationwide surveillance system established by the Taiwan Centers for Disease Control, only imported ZIKV cases were detected without local transmission. In contrast, JEV is endemic on the island; thus, a nationwide JEV pediatric vaccination program has been implemented since 1968. Periodic DENV epidemics have also occurred in Taiwan since 1981, with the largest outbreaks recorded in 2014-2015 in the southern part of the island. To investigate the antibody profile of DENV-infected patients to different flaviviruses, including ZIKV, plasma of healthy volunteers and DENV-infected febrile (DF) patients were tested for the presence of DENV, JEV, and ZIKV-neutralizing antibodies using 90% foci reduction as the cut-off (FRμNT90) (FIG. 1a). Expectedly, DF-confirmed samples had significantly elevated DENV FRμNT90 titers than the healthy donors. Only four healthy individuals showed DENV FRμNT90 titers between 50 and 80, with one donor (1413) showing DENV-1, -2, and -3 neutralizing titers, suggesting previous dengue infection or potential asymptomatic dengue infection given the overlapping residential area of the donors in southern Taiwan. Significantly elevated JEV FRμNT90 titers among the DF patients were also observed compared to the healthy controls (FIG. 1a), although thirteen healthy donors from the older age group showed varied JEV-neutralizing titers. Surprisingly, the anti-ZIKV-neutralizing titers were substantially higher among DF patients than the healthy individuals (FIG. 1a).

To distinguish between individuals immune to JEV due to vaccination or potential natural infection, the study population was divided into two age groups: 20-30 years old (post-JEV vaccination program) and 50-70 years old (pre-JEV vaccination program). Among DF patients, the JEV and ZIKV FRμNT90 geometric mean titers (GMTs) were statistically higher in the 50-70 age group (JEV=251; ZIKV=125) than in the 20-30 age group (JEV=29; ZIKV=21) (FIG. 1b). Similar antibody neutralization kinetics and titers were observed against other global or Taiwan clinical DENV isolates or the Asian and African ZIKV strains regardless of the age group. None of the donors showed neutralization against YFV.

The inventors further classified the donors to determine the impact of JEV and DENV immunity on ZIKV neutralization. Individuals from the JEV^{pos}DENV^{high} group had significantly elevated circulating ZIKV-neutralizing antibodies (GMT=84) than the others (GMT<10) for both age groups (FIG. 1c). Among the DF individuals with elevated neutralizing ZIKV titers (ZIKV^{pos}), 28 of the 31 subjects (90.3%) were from the JEV^{pos}DENV^{high} group. In contrast, only three individuals (9.68%) recorded high ZIKV neutralization titers from the other groups (FIG. 1d). The percentage of individuals with high JEV and DENV titers and consequently high ZIKV titers was statistically higher than the proportion of individuals with only one immunity, suggesting that sequential exposures to JEV and DENV could potentially induce high and cross-neutralizing antibodies against ZIKV that have yet to be encountered by the host.

ZIKV-CR huMAbs Identified from a Donor at Late Convalescence

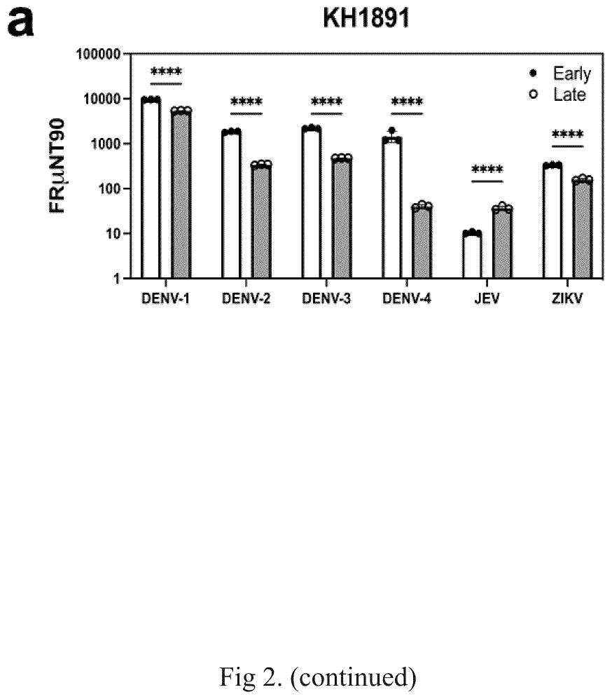

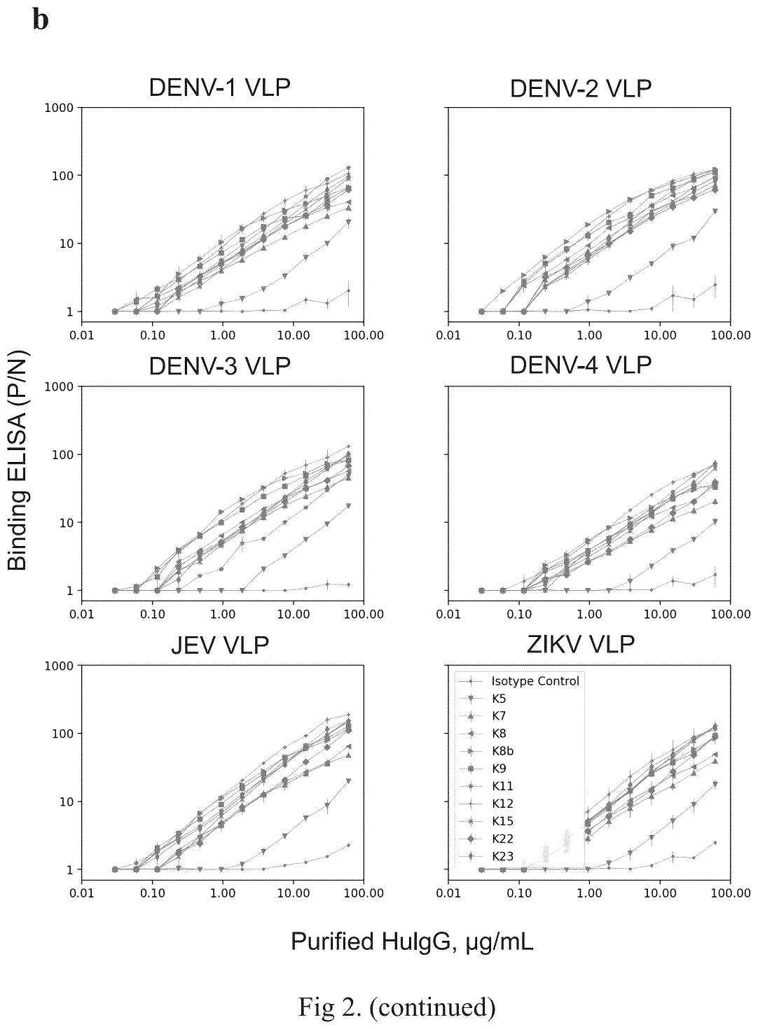

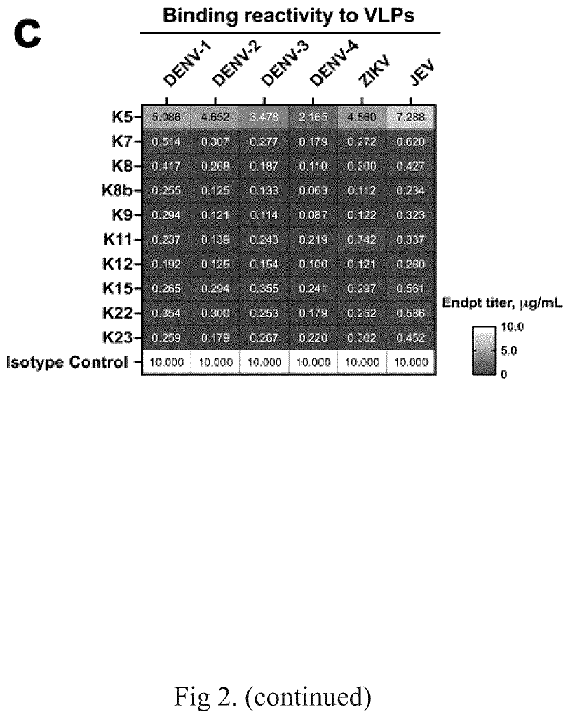

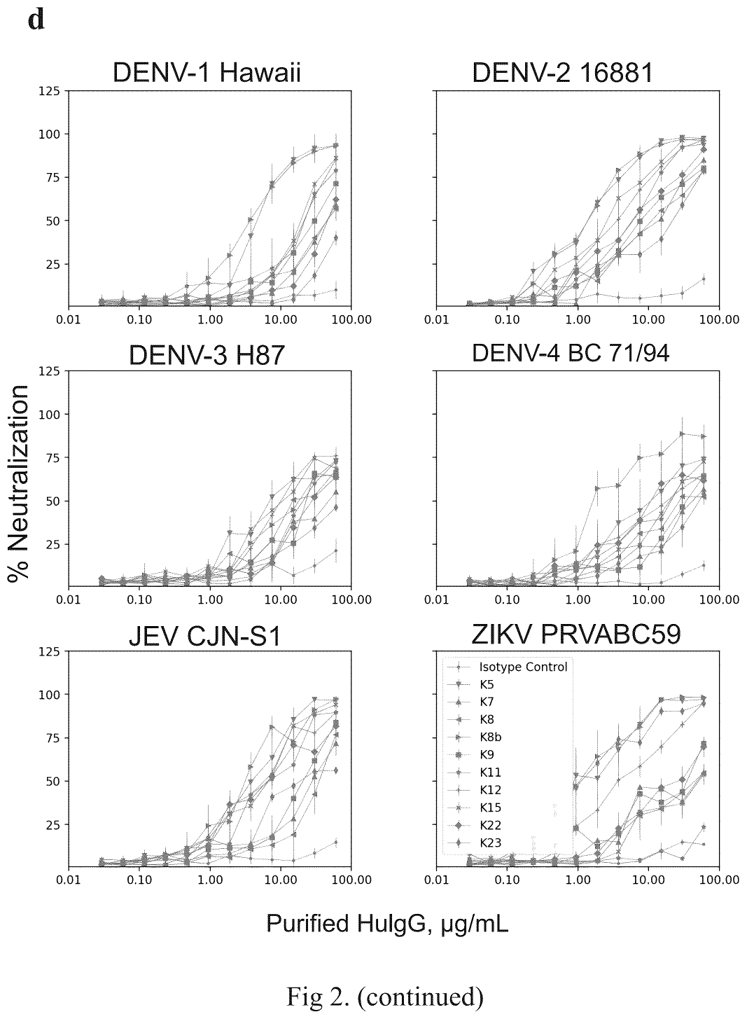

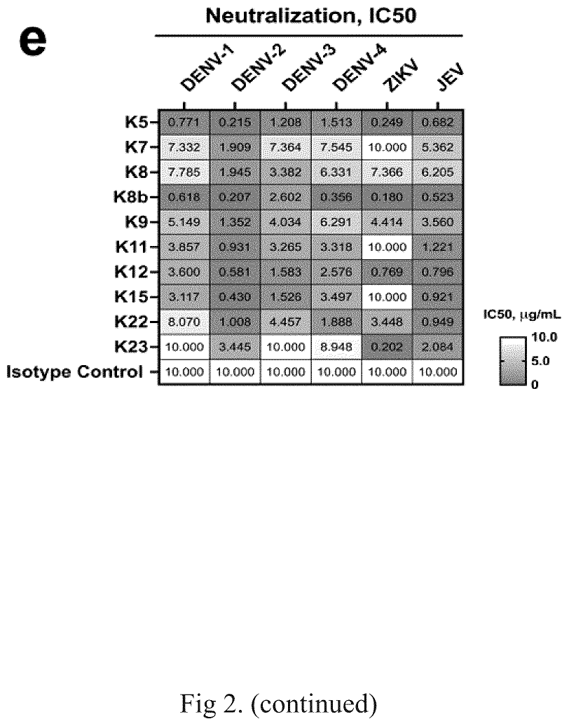

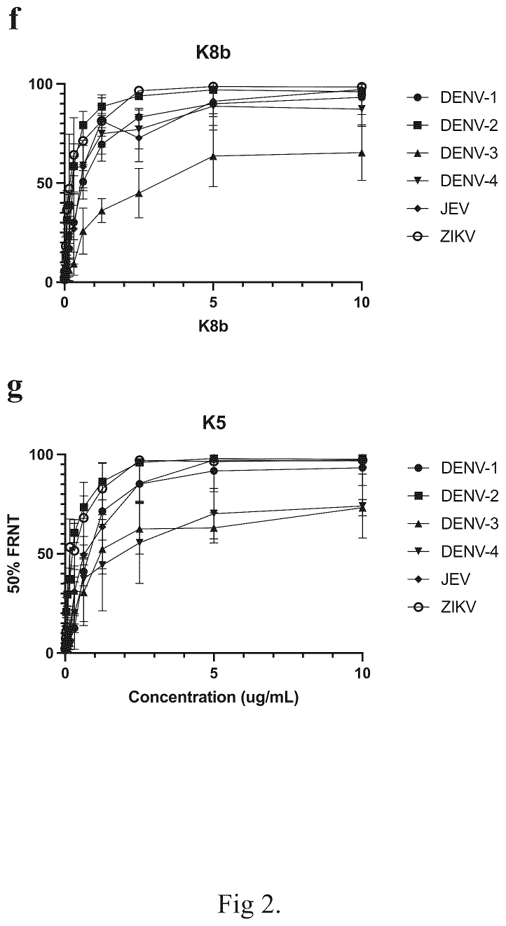

Previous longitudinal analyses suggested that short-lived but cross-neutralizing antibody responses can be generated after heterologous DENV exposures and are greatest among dengue-infected plasma samples collected during the early convalescent phase post-infection. To confirm if such cross-neutralizing ZIKV antibodies persist, the inventors successfully recalled one recovered donor (KH1891) from the same Taiwan cohort more than 18 months after the dengue infection despite the difficulty of recalling recovered donors. While JEV-neutralizing titers increased slightly, a significant decrease in DENV-1 to -4 FRμNT90 titers was observed, and ZIKV-neutralizing titers remained prominent (FIG. 2a, P<0.001).

Next, the inventors utilized the peripheral blood mononuclear cells (PBMCs) from donor KH1891 to interrogate the presence of ZIKV-specific B cells. Given the low percentage of circulating memory B cells (MBCs) in the peripheral blood at late convalescence, the inventors generated an in-house B cell sorting and co-culture strategy for ZIKV-reactive B cell clone screening to isolate huMAbs. After single-cell sorting, 1.11% of CD19⁺IgM⁻IgA⁻IgD⁻ B cells were co-cultivated with CD40L-expressing feeder cells in a medium supplemented with interleukin-2 (IL-2) and IL-21 for two weeks. Following ZIKV-reactive B cell screening, human antibody variable genes were amplified from 24 ZIKV-reactive B cell clones and expressed as full-length IgG1 with native-paired heavy and light chains. For initial crude screening, the inventors focused on broad and potent activities by ZIKV neutralization (% FRμNT50) and found that 42% (10/24) showed neutralizing activities; hence, termed as ZIKV-CR huMAbs and were expressed in large-scale, purified, and further characterized for binding and neutralization.

All ten ZIKV-CR huMAbs showed similar binding activities to all virus-like particles (VLPs) (FIG. 2b), except K5, which showed lowest binding activities to all VLPs (FIG. 2c). Of the ten, five huMAbs (K5, K8b, K12, K22, and K23) demonstrated variable yet broad and significant neutralization against three flavivirus serocomplexes; three of which (K5, K8b, and K12) showed significant half-maximal inhibitory concentrations (IC50 neutralization titers, P<0.05) against DENV-1 to -4, JEV, and ZIKV while two huMAbs (K22 and K23) displayed potent activities against selected DENV serotypes, JEV, and ZIKV (FIG. 2d). In contrast, the other five huMAbs (K7, K8, K9, K11, and K15) have restricted neutralization to DENV-1 to -4 and JEV, with K11 and K15 displaying significant IC50 titers to DENV-1 to -4 and JEV. ZIKV-CR huMAbs showed a broad range of neutralization potency (IC50 from 0.180 to 10.0 μg/mL) (FIG. 2e). Interestingly, K8b and K5, respectively, displayed the broadest and most potent neutralization (FRμNT50) against ZIKV (0.180 μg/mL; 0.249 μg/mL), DENV-2 (0.207 μg/mL; 0.215 μg/mL), DENV-4 (0.356 μg/mL), JEV (0.523 μg/mL; 0.682 μg/mL), and DENV-1 (0.618 μg/mL; 0.771 μg/mL), but moderate neutralizing activity to DENV-3 (2.60 μg/mL; 1.208 μg/mL) and against DENV-4 (1.513 μg/mL) by K5 (FIG. 2f, g).

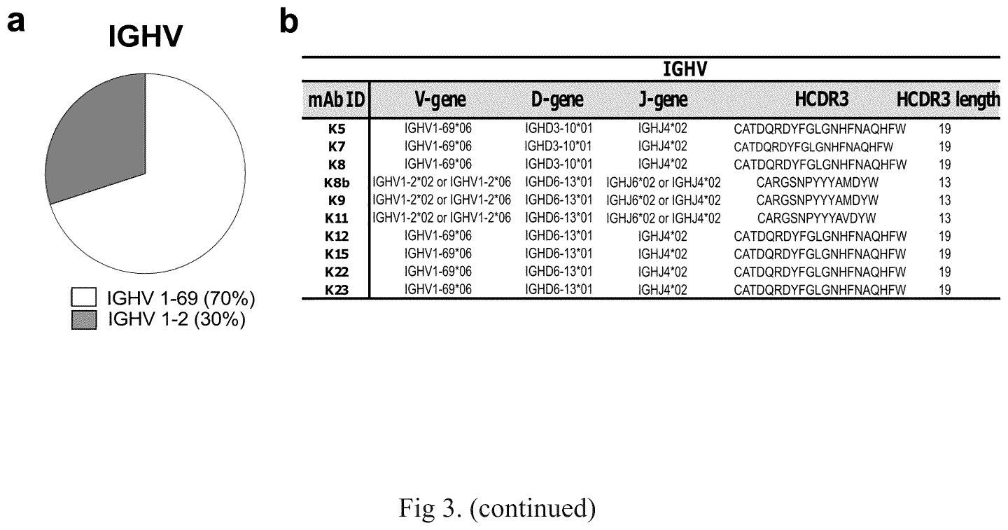

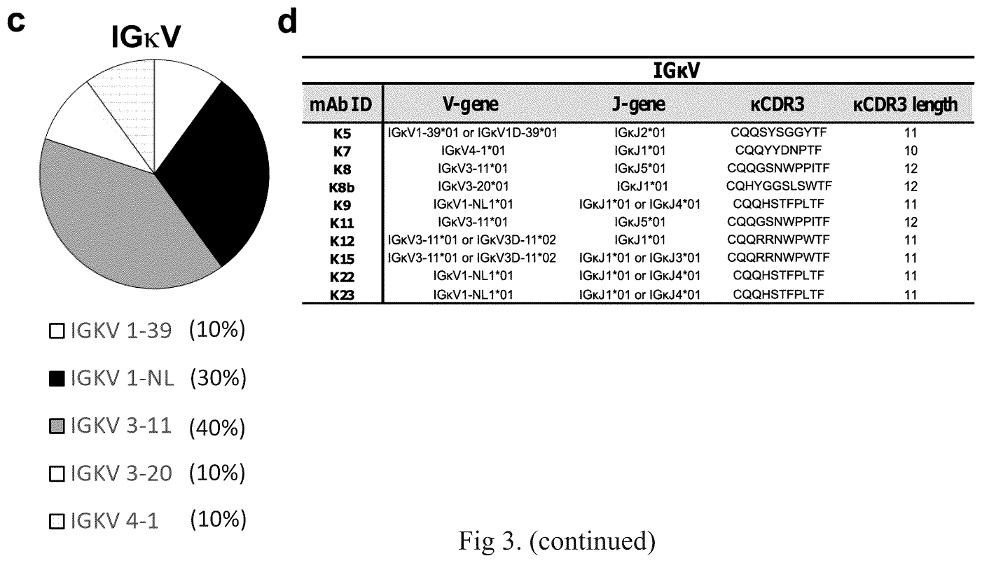

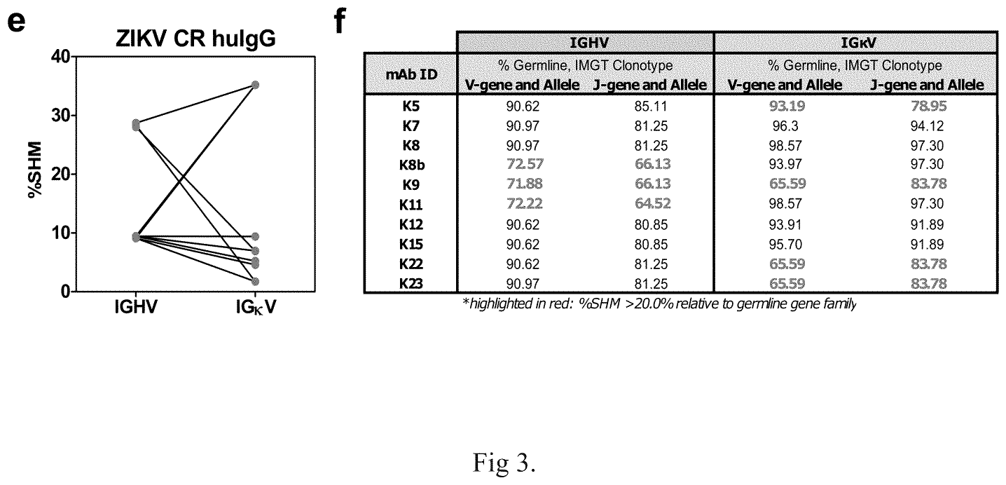

Further investigating the gene usage from the isolated huMAbs, the MBCs expressing the ZIKV-CR huMAbs utilized gene segments IGHV 1-69 (K5, K7, K8, K12, K15, K22, and K23) and IGHV 1-2 (K8b, K9, and K11) (FIG. 3a, b), compared to the non-ZIKV-CR huMAbs with broader IGHV gene assignment. The inventors also observed identical V, D, J, and heavy chain complementarity determining region 3 (HCDR3) sequences among ZIKV-CR huMAbs (FIG. 3b), suggesting a close clonal relationship of B cells expressing the broadly neutralizing antibodies. Despite heavy chain similarities, the light chain V-genes of the ZIKV-CR huMAbs are encoded by diverse germline genes, such as IGκV1, IGκV3, and IGκV4, similar to non-ZIKV-CR huMAbs, and consequently, diverse kappa light chain CDR3 (κCDR3) sequences (FIG. 3c, d). High somatic hypermutation (SHM) rates among the ZIKV-CR huMAbs in either heavy or light chain V-genes were also observed (FIG. 3e), equivalent to secondary dengue infections and influenza vaccination or infection and suggestive of enhanced affinity maturation; thus, higher relative proportion (˜80%) of dissimilar amino acids. On average, the ZIKV-CR huMAbs accumulated more non-silent nucleotide mutations in the heavy chain (n=29) than the non-ZIKV-CR huMAbs (n=20) in this study and previously known influenza-specific MBCs (n=20), with significant mutations demonstrated in HCDR2 and HFR3 domains. Although our study only included one donor, the average number of V-gene nucleotide mutations in ZIKV-CR huMAbs of KH1891 was comparable to the heavy chain V-gene mutations reported among donors with high ZIKV-neutralizing titers. Six of the ZIKV-CR huMAbs (K5, K8b, K9, K11, K22, and K23) carried the increased SHM (>20%) in either the heavy or light chain or both Ig genes, while four showed minimal changes compared to the germline sequences (FIG. 3f). Compared to the non-ZIKV-CR huMAbs with lower SHM, ZIKV-CR huMAbs showed unique heavy and light chain V(D)J rearrangements and pairing. Taken together, the ZIKV-CR neutralizing huMAbs, including K8b and K5, were isolated from the peripheral blood at the late convalescent stage and showed high degrees of non-silent SHM, suggesting the persistence and continuing maturation of the MBCs upon repeated exposures to JEV and DENV.

Structure-Guided Epitope Mapping of huMAbs K8b and K5

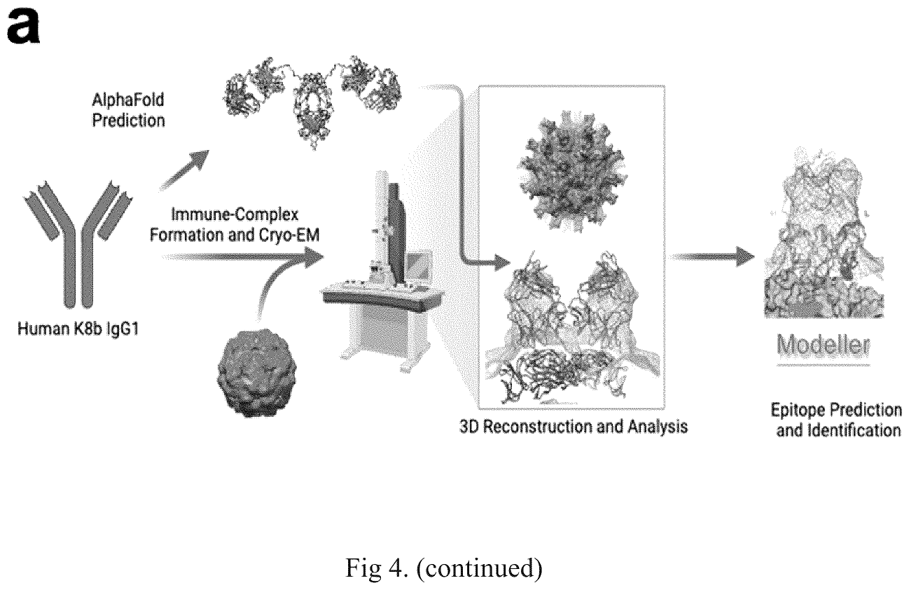

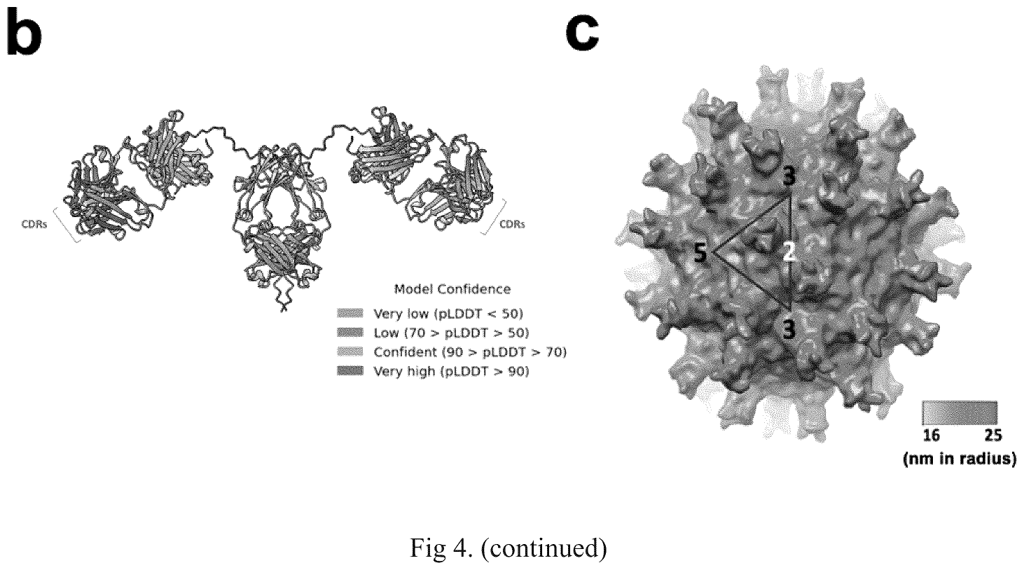

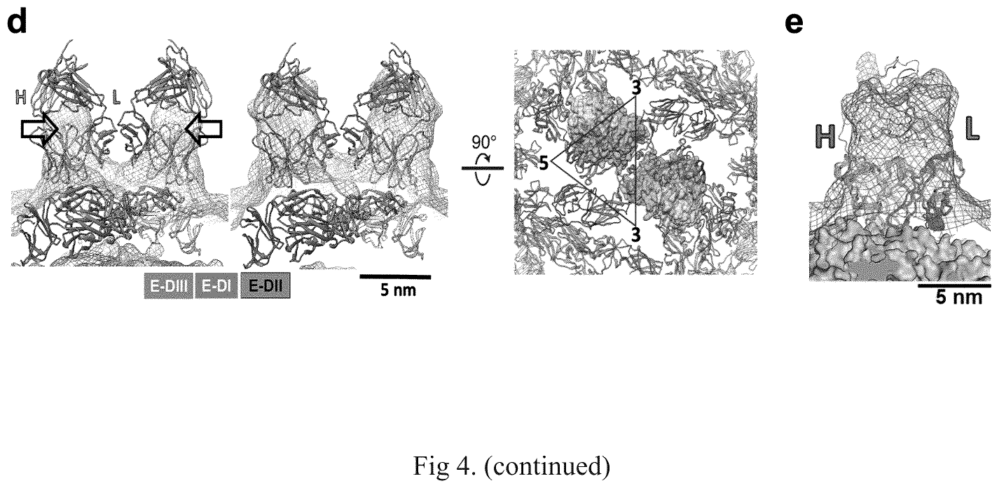

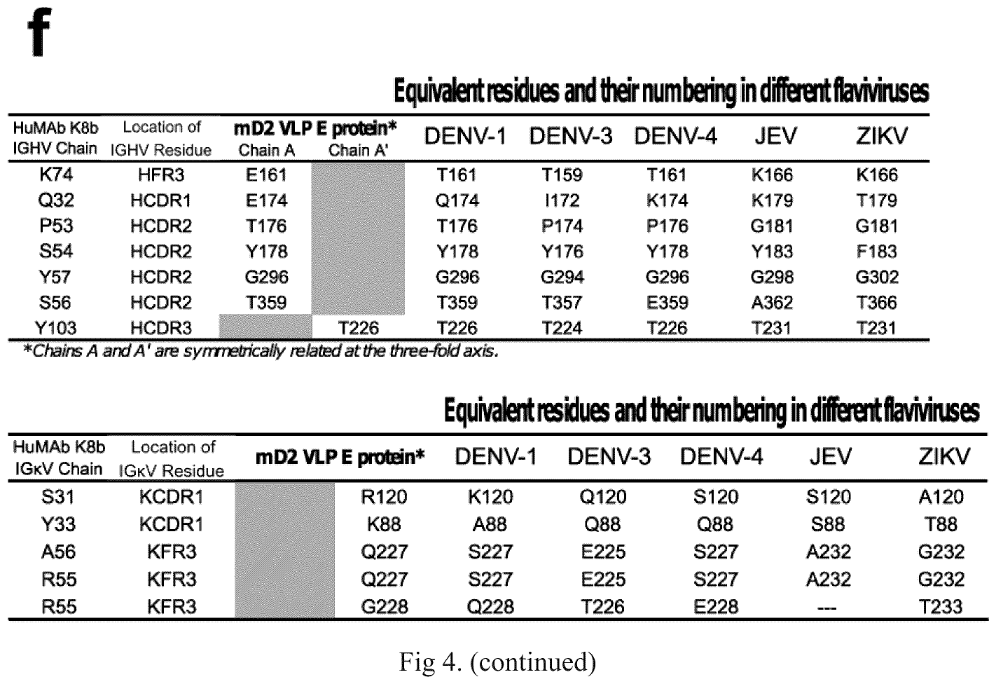

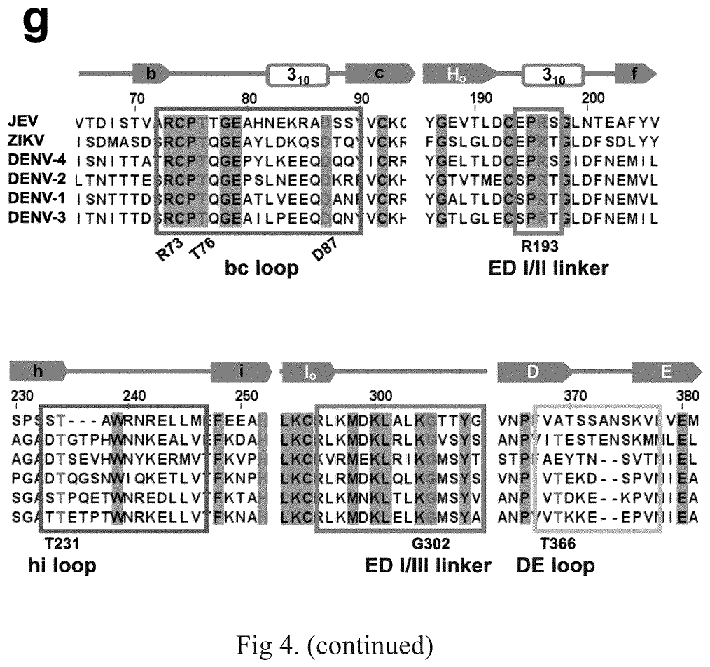

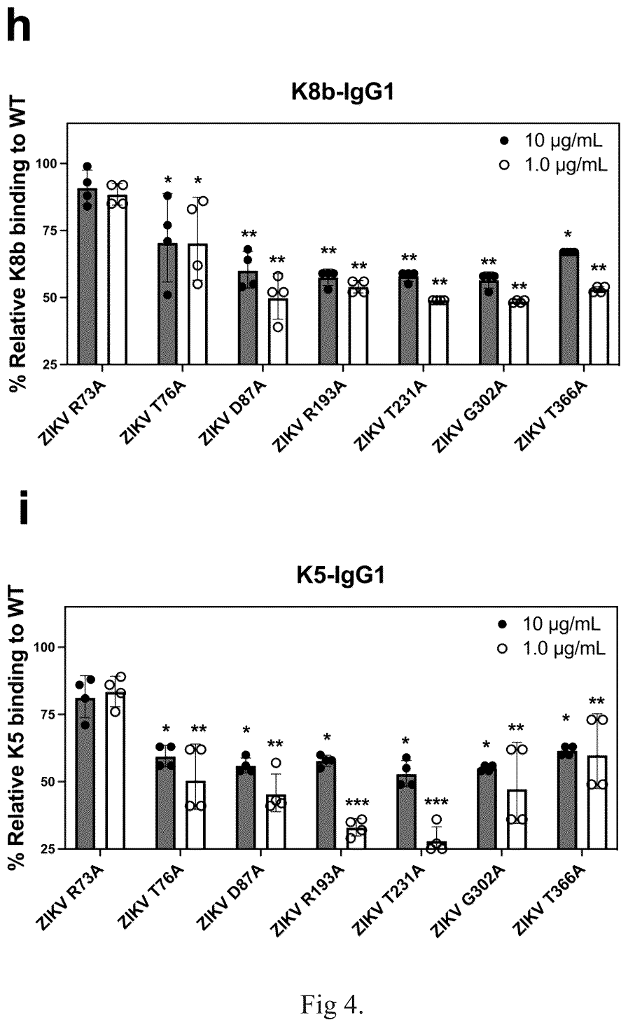

The inventors chose K8b for epitope characterization due to its broad neutralization activities and higher expression level. Instead of performing random shotgun mutation on the complete E gene, the inventors developed a structure-based strategy using a previously generated mature form virus-like particles of dengue virus serotype 2 (mD2VLP) to form an immune complex with K8b-IgG1 (FIG. 4a-c). The cryo-EM micrographs of the mD2VLP-K8b-IgG1 immune complex revealed the heterogeneity in particle size, which the inventors attributed to the variability of mD2VLPs. The inherent heterogeneity of mD2VLPs and antibody hinges made improving structure resolution challenging. Nevertheless, the structure of the mD2VLP-IgG1 immune complex with unresolved Fc was determined (FIG. 4c). The resolution of this structure was calculated to be 16.3 Å. Details of the cryo-EM data collection, refinement, and validation statistics were shown in Table 1. The surface-rendered structure at a high threshold (1.2σ) clearly demonstrated the separation of variable domains from constant domain within the density map, greatly assisting in the orientation of the F(ab) fragments (FIG. 4d). A more precise fitting was achieved using HEX, which calculated the binding energy and determined the most probable binding orientation of the F(ab) fragment with another molecule (see Methods). To elucidate how the F(ab) fragments were coupled, a more permissive mask filtering approach was implemented during the reconstruction process. Notably, the configuration of the Fc region dictated the K8b-IgG1 bivalent arms, which bound separately to different E-dimer units, suggesting that K8b-IgG1 may function not only as an interdomain antibody but also an inter-dimer antibody (FIG. 4d).

TABLE 5
Cryo-EM data collection, refinement, and validation statistics.

mD2VLP-K8b-IgG1 immunocomplex
(EMDB-36408)

Data collection and processing

| | |
|---|---|
| Magnification | 15,000 |
| Voltage (kV) | 200 |
| Electron exposure (e−/Å) | 32 |
| Defocus range (μm) | 2.3-4.2 |
| Pixel size (Å) | 4 |
| Symmetry imposed | icos |
| Initial particle images (n) | 2035 |
| Final particle images (n) | 1246 |
| Map resolution (Å) | 16.3 |
| FSC threshold | 0.143 |
| Map resolution range (Å) | 16.0-19.9 |

Refinement

| | |
|---|---|
| Initial model used (PDB code) | n.a. |
| Model resolution (Å) | n.a. |
| FSC threshold | |
| Model resolution range (Å) | n.a. |
| Map sharpening B factor (Å) | n.a. |
| Model composition | n.a. |
| Non-hydrogen atoms | |
| Protein residues | |
| Ligands | |
| B factors (Å) | n.a. |
| Protein | |
| Ligand | |
| RMS deviations | n.a. |
| Bond lengths (Å) | |
| Bond angles (°) | |
| Validation | n.a. |
| MolProbity score | |
| Clashscore | |
| Poor rotamers (%) | |
| Ramachandran plot | n.a. |
| Favored (%) | |
| Allowed (%) | |
| Disallowed (%) | |

*Refinement was performed by EMAN2 software. The solved cryo-EM structure had suboptimal resolution (16.0-19.9 Å); thus, the requested detailed structural parameters could not be provided with accuracy.
*n.a. answers were not available.

The resolution of the K8b-IgG1 immune complex was further enhanced to 12 Å using MODELLER to map the potential interacting residues between the E proteins on an mD2VLP and the heavy or light chain of K8b-IgG1 (FIG. 4e, f). Based on the predicted interacting residues on the E protein of DENV-2, seven residues spanning across domains I, II, and III were mutated into alanine on ZIKV VLP based on the criteria described in Methods (see SDM and epitope mapping experiments section), such as R73, T76, and D87 on the be loop of domain I, R193 on domain I and II linker region, T231 on the hi loop of domain II, G302 on domain I and III linker region, and T366 on DE loop of domain III (FIG. 4g). The recognition of K8b-IgG1 to D87A, R193A, T231A, G302A, and T366A mutant antigens were significantly reduced by as much as 50% relative to ZIKV WT VLP (FIG. 4h). In contrast, K8b and K5 binding to residue 101-mutated VLPs were unaffected. The overall binding reactivities of K5-IgG1 to most mutant ZIKV VLPs were similar to K8b-IgG1 except for two residues, ZIKV R193A and ZIKV T231A. Mutation of R193 and T231 showed abrogation of K5 binding by 70% (FIG. 4i), indicating substantial functional contribution of both residues to K5 and ZIKV interactions.

To validate if antibody geometry could affect the binding and avidity to putative interdomain or inter-dimer epitopes on different E dimers, the full-length bivalent IgG1 of K8b (K8b-IgG1) was engineered and expressed as bivalent K8b-F(ab′)₂ and monovalent K8b-F(ab) (FIG. 5a-d). Parallel binding and neutralization experiments were performed on K8b-IgG1, K8b-F(ab′)₂, and monovalent K8b-F(ab) against ZIKV, JEV, and the four DENV serotypes. Bivalent K8b-IgG1 and K8b-F(ab′)₂ have similar neutralization against DENV-1, DENV-2, DENV-4, ZIKV, and JEV (FRμNT50: 7-17 nM), but K8b-F(ab′)₂ showed a slight improvement in neutralization against DENV-3 (FRμNT50: 26-50 nM) (FIG. 5e). Although both VLP and virions form E-dimer on the surface of the icosahedral sphere, the observed VLP binding kinetics for K8b-IgG1 and K8b-F(ab′)₂ were consistent with neutralization for DENV-2 and ZIKV only but not for JEV, DENV-1, DENV-3, and DENV-4 VLPs probably due to the difference in geometry between VLP (T=1) and virion (T=3) (FIG. 5f). Unlike K8b-F(ab′)₂, significantly reduced neutralizing activity to all six viruses was observed for K8b-F(ab) except for DENV-2, which retained moderate neutralization (FRμNT50: 37 nM). Similarly, monovalent K8b-F(ab) showed reduced binding to all VLPs with a significant drop in binding to DENV-3, DENV-4, JEV, and ZIKV VLPs. These data show that K8b could occupy quaternary epitopes on the virion surface and confirm that antibody geometry impacts the observed antigen-dependent pattern of binding and neutralization potency of K8b, explaining its potent and broad neutralizing activities.

Heterogeneous MBC Recall Responses after mD2VLP Stimulation in JEV VLP-Primed Mice

To explore the B cell immune responses in vivo mimicking the sequential exposures of JEV and DENV in human infection, the inventors conducted a prime-boost immunization study wherein mice were immunized with mD2VLP or JEV VLP antigens and evaluated the post-vaccination antibody response for neutralization against homologous and heterologous flavivirus serocomplexes (FIG. 6a). Although mice from groups 3-5 which received the heterologous VLPs showed moderate (FRμNT50 GMT: 10-200) polyclonal responses against the four DENV serotypes and JEV, the ZIKV-CR neutralizing activities of these animals were primarily low (FRμNT50 GMT: 10-60) and not significant. Notably, homologous JEV VLP prime-boost immunization consistently induced relatively higher neutralization titers not only to the homologous JEV (FRμNT50 GMT=164) but also to the heterologous flavivirus serocomplexes not encountered by the vaccinated mice (FRμNT50 GMT, DENV−1=115; DENV-2=605; DENV-3=961, DENV-4=603) and recorded the highest anti-ZIKV titers among all groups (FRμNT50 GMT=64).

To further investigate the MBC clones established by homologous JEV VLP prime-boost immunization, two of the most responsive (FRμNT50>120) mice in this group were selected for generating hybridomas using homologous or heterologous antigens, JEV VLP or mD2VLP, respectively, to stimulate MBC proliferation followed by immediate fusion with murine myeloma cells (FIG. 6b). Enzyme-linked immunosorbent assay (ELISA) binding activity showed that polyclonal hybridomas generated using JEV VLP for stimulation were all JEV-specific (FIG. 6c, left). Seventy-one percent of the 52 monoclones isolated from the 3D2 polyclone secreted JEV-neutralizing mAbs with varying levels of potency at IC50 as strong, moderate, and weak (<1, 1-10, and >10 μg/mL, respectively) (FIG. 6c, right). On the contrary, polyclonal hybridomas generated from the same homologous JEV VLP prime-boost mice but stimulated by heterologous mD2VLP antigens secreted antibodies CR to JEV, DENV, and ZIKV VLPs (FIG. 6d). Fifty-eight percent of the 36 monoclones isolated from the 5D5 polyclone secreted cross-neutralizing mAbs against all six flaviviruses, including JEV, ZIKV, and DENV-1 to -4, while the rest secreted murine mAbs only cross-neutralizing sub-group (FIG. 6e). Most of these mAbs exhibited moderate to weak cross-neutralizing activities (FIG. 6f). Notably, all 52 mAbs (or 59%) from the JEV VLP stimulation strategy belong to the IgG1 isotype, whereas 36 mAbs (or 41%) from mD2VLP stimulation all belong to the IgG3 isotype (FIG. 6g), demonstrating that the heterologous VLP vaccination model induced the IgG3-focused recall responses. Thus, these findings suggest that heterogeneous MBC populations recognizing different flaviviruses can be established from JEV prM/E VLP antigens and recalled upon subsequent heterologous VLP antigen exposure, resulting in IgG isotype-dependent responses with varying neutralizing activities.

Given the IgG3-induced CR response in mice after heterologous antigen stimulation, the inventors next examined if the cross-neutralization activities against ZIKV previously observed in polyclonal human sera were associated with IgG3-specific antibodies by performing a total human IgG3 GAC ELISA and antigen-specific IgG3 capture ELISA. Eighteen of the 31 individuals (58%) with ZIKV VLP-reactive IgG responses showed ZIKV VLP-binding IgG3 antibodies despite the similar total human IgG (P=0.2024) and relative IgG3-specific antibodies (P=0.1385) among the healthy controls and DF patients. Notably, the inventors found a significant and positive correlation between the ZIKV VLP-binding IgG3 subclass and ZIKV neutralization titers (Pearson's correlation, r=0.4; Wilcoxon matched-pairs signed rank test, W=0, P<0.05;). The inventors hypothesized that the low neutralization activity of K8b-IgG1 against DENV-3 observed in this study could be enhanced when expressed as IgG3. Thus, to explore the possible contribution of IgG3 in broad neutralization, one of the broadly neutralizing antibodies, K8b, was expressed as IgG3. The purity of these preparations was assessed by SDS-PAGE under reducing and nonreducing conditions showing the expectedly 10-kDa higher molecular weight of K8b-IgG3 than K8b-IgG1. Though K8b-IgG3 showed slightly lower neutralization potency against DENV-3 compared with K8b-IgG1 (P<0.05), no differences were observed in neutralization activities between K8b isotypes against the other prototype strains of DENV-1, -2 and -4, JEV, and ZIKV, as shown by the statistically similar geometric mean IC50 neutralization titers for all virions, suggesting that an exchange of the Fc region contributed minimally in the neutralization potency of K8b. The inventors re-evaluated the natural immunoglobulin isotype of K8b from the original ZIKV-reactive B cell clone, where it was isolated using human Fc region-specific primers, and confirmed that K8b naturally existed as an IgG1 molecule.

### DISCUSSION

A few studies investigated the interplay of flavivirus immunity in the context of JEV, which is endemic in Southeast Asia; however, these studies focused on animal immunization or ex vivo stimulation using PBMCs from JEV-experienced donors. Contrary to the local ZIKV transmission in neighboring Asian countries, Taiwan, situated in East Asia, detects only imported ZIKV cases without any local transmission. To exclude the possibility of subclinical silent transmission of ZIKV, the seroprevalence studies conducted in southern Taiwan support very few or no local transmission of ZIKV; thus, providing a unique cohort setting to investigate CR antibody profile after sequential exposures to JEV and DENV. Our study provided serological evidence of a robust anti-ZIKV antibody response elicited by JEV-primed individuals who encountered a subsequent natural DENV infection. These serological findings were further confirmed by the isolation of CR huMAbs and heterologous antigen stimulation of the mice primed by JEV antigens. Previous studies showed that sequential exposures to heterologous DENV serotypes generated potent broadly neutralizing antibodies with longevity to ZIKV, reduced ZIKV symptoms in a pediatric cohort in Nicaragua and lowered the risk of ZIKV infections in a large prospective Brazilian cohort. While these studies highlighted the importance of infection history on flavivirus immunity, they were mainly conducted in countries without reported JEV circulation. Our study demonstrated a promising proof of concept for generating broadly neutralizing antibodies after sequential JEV and DENV exposures.

Our current knowledge of cross-immunity to ZIKV is mainly derived from infection of DENV or other flaviviruses such as WNV and YFV and is limited to the investigation of the interrelationships of two flavivirus serocomplexes. The higher ZIKV-neutralizing titers the inventors observed among the older dengue-infected individuals in our study could suggest that pre-existing MBCs acquired from natural JEV exposure were long-lived and more persistent than those derived from vaccination. Alternatively, it could be due to the repeated exposures to geographically co-circulating flaviviruses over a lifetime in the older age group. In prior age-stratified seroprevalence studies, the accumulation of multitypic dengue and JEV immunity increased with age, with dengue-immune adults (≥30 years old) showing reduced susceptibility to secondary infections than monotypically exposed children and adolescents under 15 years. The inventors cannot completely rule out the possibility of weakly neutralizing antibodies being linked to disease severity among individuals with prior secondary DENV exposures. However, passive transfer of cross-neutralizing antibodies from JEV-vaccinated mice failed to induce antibody-dependent enhancement (ADE) in vitro or affect ZIKV infection or pathogenesis in mice, although these antibodies were low or undetectable and highly cross-reactive. Finally, the high endemicity and geographic co-circulation of mosquito-borne flaviviruses in Asia, such as DENV and JEV, could confer apparent cross-protection provided by B and T cell cross-immunity, thus partly explaining the silent ZIKV transmission or low ZIKV incidences in the region. Similarly, the lack of WNV outbreaks in Latin America was also hypothesized to be due to the co-circulation of related flaviviruses endemic in the Americas.

Factors governing the germinal center reaction, affinity maturation, and the induction of circulating MBCs and long-lived plasma cells are complex and remain an active area of research. Previous studies showed that JEV vaccination induces long-lived and protective neutralizing antibodies and memory cytotoxic T lymphocytes in mice and children from 1 year to 5 years after initial JEV administration. One-time passive transfer of anti-JEV-neutralizing antibodies was also more protective against homologous challenge than heterologous viruses; however, repeated homologous JEV exposures trigger an extensive MBC response to generate neutralizing antibodies and increased levels of JEV-primed CD8+ T cells, thus, protecting JEV-infected mice from lethality. In this study, homologous JEV VLP prime-boost immunization effectively induced homologous and heterologous in vitrocross-neutralizing responses, which were sufficient to form clonally diverse MBCs, waiting to be recalled for clonal expansion after DENV exposure. Similarly, the mice study with sequential WNV and JEV exposures suggests that flavivirus-specific MBCs bypass the germinal center in recall response, whose activity depends on the initial clonal diversity of MBCs derived from the initially encountered antigens. Other factors, such as the interval of priming and boosting, the order of heterologous exposures, or the types of antigens used and the duration after the germinal center formation before encountering heterologous antigens could also complicate the outcome of heterologous immunity. Further experimental work is needed to determine whether prime-boost immunization of heterologous antigens in mice could also induce similar clonally diverse MBCs in the context of flavivirus infections.

Recent studies in human immunodeficiency virus (HIV) suggested that IgG3 played a vital role in broadly neutralizing antibody responses due to its longer hinge. Our findings in the mice immunization study showing that the CR murine monoclonal antibodies isolated from heterologous antigen stimulation are all IgG3 prompted us to examine the IgG3 profile among our DF patients. The inventors further engineered K8b-IgG1 as IgG3 to see if the exchange of Fc region could enhance the moderate neutralizing activity against DENV-3. Despite the significantly positive correlation between ZIKV-VLP-specific IgG3 binding activity and ZIKV-neutralizing antibody titer, no difference in neutralizing activities against the five different flaviviruses was observed between K8b-IgG1 and K8b-IgG3 except DENV-3. As K8b is naturally expressed as IgG1, it may not be structurally advantageous in neutralization when expressed as IgG3. Furthermore, the isotypes of the other ZIKV-CR huMAbs from the original B cell clone remain to be identified. The importance of IgG3 in boosting neutralization against various flaviviruses requires future studies, and the potential impact of IgG3 in mediating Fe effector functions observed elsewhere will augment our knowledge of the functional relevance of Ig subclasses in the context of heterologous flavivirus infections.

The natural occurrence of huMAbs with high cross-neutralization potency is rare, and only a few well-characterized antibodies generated from vaccinations or heterologous infections of humans with prior flavivirus immunity have been described. Of which, huMAbs MZ20 and MZ54/56 are relevant to the current work since they potently neutralize flaviviruses from three distinct serocomplexes, such as DENV-1 to -4, ZIKV, and JEV or WNV. Other known huMAbs, such as SigN-3C, J8/J9, or F25.S02, were excluded for comparison because only their neutralizing activities against two serocomplexes, DENV and ZIKV, were reported. It is unknown if they could further neutralize a third serocomplex, such as JEV, and might not represent a fair comparison to our huMAbs in the context of our current hypothesis. The inventors initially hypothesized that the potential epitopes of our broadly neutralizing huMAb (bn-huMAb) centered on the conserved regions of flaviviruses from different complexes and spread among the surface accessible loops, such as bc, hi, ij, or fusion loop (FL), previously observed in other bn-huMAbs, MZ20, MZ54/56, and 1C19. On the other hand, K8b and our bn-huMAbs showed lower potency compared to known E DIII lateral ridge-recognizing bn-huMAbs capable of picomolar level-neutralization, such as ZIKV-116, 1C11, ZK004/006, and MZ4. The weaker neutralizing activities against DENV-3 by K8b-IgG1 could be due to a subset of amino acids within the E glycoprotein that is conserved among JEV, ZIKV, and other DENV serotypes but not in DENV-3 including, histidine (H158) and proline (P164/166/171) residues, both located at the glycan loop (F₀ ß-strand). The absence of H158 and the change of proline to serine in DENV-3 possibly affected the glycosylation and conformational landscape-determined epitope accessibility on the surface of DENV, ZIKV, and JEV. Further superimposition of the footprint of K8b on each VLP showed that the electrostatic potential of its light chain on DENV-3 is neutral, indicating lowered binding affinity and, thus, reduced DENV-3 neutralization.

The bn-huMAbs isolated in our study could originate from pre-existing type-specific MBCs that underwent multiple rounds of selection and SHM. Firstly, the B cell repertoire from one donor initially primed with JEV and with recent exposure to DENV involves clonally distinct B cells producing two populations of huMAbs: ZIKV- and the non-ZIKV-CR huMAbs. Among the ZIKV-CR huMAbs, these clonally related antibody sequences showed that the group of antibodies with increased SHMs, such as K5 and K8b, are usually potent neutralizers and complex-specific. The development of these CR B cells may be driven by clonal selection during the primary encounter with JEV and the subsequent recall and somatic evolution of CR MBC repertoire upon re-exposure to antigenically similar DENV through natural infection. Secondly, our murine MBC stimulation study on JEV VLP-immune mice successfully isolated MAbs recognizing flaviviruses from different serocomplexes, which confirmed that the heterogeneous MBC populations could be established from JEV prM/E VLP antigens and subsequently recalled after encountering a heterologous VLP antigen from a different serocomplex. The reactivation and recall of MBC responses upon antigen re-encounter have been initially observed in the T cell immune repertoire, highlighting the concerted action of the T cell and B cell immune repertoire in the development and mediation of the broad humoral response after multiple flavivirus exposures. Thirdly, the higher IGHV 1 gene usage in donor KH1891, expressing both the ZIKV- and non-ZIKV-CR huMAbs, is consistent with recurrent lineage usage of VH1/Vκ1 cross-neutralizing DENV-1 and ZIKV. Despite the similarity in HCDR3 and V(D)J genes among these bn-huMAbs from one donor, the specific target binding is an outcome of unique rearrangements and VL pairing and explains the variability in the observed neutralization. Finally and intriguingly, the isolation of huMAb K5 in our study, which utilized similar heavy (IGHV 1-69) and light chain genes (IGκV 1-39), as a DENV-3 serotype-specific neutralizing huMAb, 5J7. Both antibodies showed varied HCDR3 sequences and lengths, with K5 showing longer HCDR3 than 5J7 by five residues. Interestingly, amino acid alignment showed that 5J7 had 16 residues that were dissimilar from K5, which spanned across the variable region, suggesting the bn-huMAbs could originate from a serotype-specific B cell that underwent the classical recombination event and then acquired further SHM and insertions at the HCDR3 through selection. All these events indicate that the ZIKV-CR huMAbs the inventors isolated in this study originated from two distinct B cell lineages, meaning separate pathways of MBC development after heterologous antigen stimulation, supporting the importance of flavivirus exposure history on the host humoral response.

Overall, the inventors demonstrated in humans and mice how the pre-immunity to flaviviruses would shape the humoral response against an antigen from a different serocomplex to which the host has no previous exposure history, resulting in a unique subpopulation of antibodies with broad and potent cross-neutralizing activities. These findings not only enhance our understanding of the quality of humoral response induced in humans after sequential exposures to flaviviral infections but also have implications for alternative vaccination strategies, especially in DENV-endemic regions where ZIKV is known to co-circulate geographically.

What is claimed is:

1. A neutralizing antibody for flaviviruses, comprising:
(1) a heavy chain complementary determining region 1 (HC CDR1) set forth as SEQ ID NO: 1,
a heavy chain complementary determining region 2 (HC CDR2) set forth as SEQ ID NO: 2,
a heavy chain complementary determining region 3 (HC CDR3) set forth as SEQ ID NO: 3,
a light chain complementary determining region 1 (LC CDR1) set forth as SEQ ID NO: 4,
a light chain complementary determining region 2 (LC CDR2) set forth as SEQ ID NO: 5, and
a light chain complementary determining region 3 (LC CDR3) set forth as SEQ ID NO: 6;
(2) a HC CDR1 set forth as SEQ ID NO: 7,
a HC CDR2 set forth as SEQ ID NO: 8,
a HC CDR3 set forth as SEQ ID NO: 9,
a LC CDR1 set forth as SEQ ID NO: 10,
a LC CDR2 set forth as SEQ ID NO: 11, and
a LC CDR3 set forth as SEQ ID NO: 12;
(3) a HC CDR1 set forth as SEQ ID NO: 7,
a HC CDR2 set forth as SEQ ID NO: 8,
a HC CDR3 set forth as SEQ ID NO: 9,
a LC CDR1 set forth as SEQ ID NO: 13,
a LC CDR2 set forth as SEQ ID NO: 14, and
a LC CDR3 set forth as SEQ ID NO: 15; or
(4) a HC CDR1 set forth as SEQ ID NO: 7,
a HC CDR2 set forth as SEQ ID NO: 8,
a HC CDR3 set forth as SEQ ID NO: 9,
a LC CDR1 set forth as SEQ ID NO: 16,
a LC CDR2 set forth as SEQ ID NO: 17, and
a LC CDR3 set forth as SEQ ID NO: 18.

2. The neutralizing antibody of claim 1, wherein the neutralizing antibody comprises a heavy chain variable domain (V_{H}) that is at least 85% identical to the amino acid sequence of SEQ ID NO: 19, and/or a light chain variable domain (V_{L}) that is at least 85% identical to the amino acid sequence of SEQ ID NO: 20.

3. The neutralizing antibody of claim 1, wherein the neutralizing antibody comprises a V_{H} that is at least 85% identical to the amino acid sequence of SEQ ID NO: 21, and/or a V_{L} that is at least 85% identical to the amino acid sequence of SEQ ID NO: 22.

4. The neutralizing antibody of claim 1, wherein the flaviviruses comprises Dengue fever virus serotype 1 (DENV-1), Dengue fever virus serotype 2 (DENV-2), Dengue fever virus serotype 3 (DENV-3), Dengue fever virus serotype 4 (DENV4), Japanese encephalitis virus (JEV), and/or Zika virus (ZIKV).

5. The neutralizing antibody of claim 1, wherein the neutralizing antibody is capable of neutralizing more than one subtypes of the flaviviruses.

6. The neutralizing antibody of claim 1, wherein:
(a) the neutralizing antibody binds within residues 72-89, 98-111, 186-190, 224-237, 243-249, 286-299, and/or 357-364 of the DENV-1 comprising the amino acid sequence of SEQ ID NO: 23,
(b) the neutralizing antibody binds within residues 72-89, 98-111, 186-190, 224-237, 243-249, 286-299, and/or 357-364 of the DENV-2 comprising the amino acid sequence of SEQ ID NO: 24,
(c) the neutralizing antibody binds within residues 72-89, 98-111, 184-188, 222-235, 241-247, 284-297, and/or 355-362 of the DENV-3 comprising the amino acid sequence of SEQ ID NO: 25, or
(d) the neutralizing antibody binds within residues 72-89, 98-111, 186-190, 225-238, 252-258, 287-300, and/or 358-365 of the DENV-4 comprising the amino acid sequence of SEQ ID NO: 26.

7. The neutralizing antibody of claim 1, wherein the neutralizing antibody binds within residues 72-89, 98-111, 191-195, 229-239, 245-251, 288-301, and/or 360-369 of the JEV comprising the amino acid sequence of SEQ ID NO: 27.

8. The neutralizing antibody of claim 1, wherein the neutralizing antibody binds within residues 72-89, 98-111, 191-195, 229-242, 248-254, 292-305, and/or 364-373 of the ZIKV comprising the amino acid sequence of SEQ ID NO: 28.

9. The neutralizing antibody of claim 1, wherein the neutralizing antibody is a monoclonal antibody.

10. The neutralizing antibody of claim 1, wherein the neutralizing antibody is a full-length antibody or an antigen binding fragment thereof.

11. The neutralizing antibody of claim 10, wherein the full-length antibody comprises an IgG molecule.

12. The neutralizing antibody of claim 10, wherein the antigen binding fragment of the neutralizing antibody is selected from the group consisting of F(ab′)2, Fab′, Fab, Fv, single domain antibody (VHH), and scFv.

13. A method for producing a neutralizing antibody, comprising:
(a) providing a mammal animal;
(b) delivering an antigen to the mammal animal, wherein the antigen comprises Japanese encephalitis virus (JEV) antigen, Dengue fever virus (DENV) antigen, and/or a combination thereof;
(c) selecting a B cell that expresses the neutralizing antibody from the mammal animal, wherein the neutralizing antibody is capable of neutralizing Zika virus (ZIKV); and
(d) obtaining the neutralizing antibody from the B cell.

14. The method of claim 13, wherein the mammal animal comprises mouse, rat, rabbit, monkey, chimpanzee, and/or human.

15. The method of claim 13, wherein in the step (b), the JEV antigen is delivered prior to the DENV antigen to the mammal animal.

16. The method of claim 13, wherein the antigen is delivered at levels sufficient to induce an immune response of the mammal animal.

17. The method of claim 13, wherein the JEV antigen comprises JEV and/or JEV vaccine.

18. The method of claim 17, wherein the JEV vaccine comprises inactivated Vero cell culture derived JE vaccine (JE-VC), inactivated mouse brain derived JE vaccine (JE-MB), primary hamster kidney cell derived, attenuated JE vaccine, and/or live attenuated chimeric JE vaccine.

19. The method of claim 13, wherein the DENV antigen comprises DENV vaccine, DENV serotype 1 (DENV-1), DENV serotype 2 (DENV-2), DENV serotype 3 (DENV-3), and/or DENV serotype 4 (DENV-4).

20. The method of claim 19, wherein the DENV vaccine comprises live attenuated dengue vaccine.

21. The method of claim 13, wherein in the step (d), the neutralizing antibody is obtained from the B cell, or is obtained by expressing an isolated DNA encoding the neutralizing antibody derived from the B cell.

22. The method of claim 21, wherein the isolated DNA encodes a full-length antibody of the neutralizing antibody or an antigen binding fragment thereof.

23. The method of claim 22, wherein the full-length antibody comprises an IgG molecule.

24. The method of claim 22, wherein the antigen binding fragment of the neutralizing antibody is selected from the group consisting of F(ab′)2, Fab′, Fab, Fv, single domain antibody (VHH), and scFv.

25. A pharmaceutical composition comprising the neutralizing antibody of claim 1, optionally together with a pharmaceutically acceptable carrier, diluent or excipient.

26. A method of treating or preventing a flaviviruses infection in a subject, comprising administering to the subject an effective amount of the pharmaceutical composition according to claim 25 under conditions effective to neutralize the flaviviruses.

27. The method of claim 26, wherein the flaviviruses comprises Dengue fever virus (DENV-1), Dengue fever virus serotype 2 (DENV-2), Dengue fever virus serotype 3 (DENV-3), Dengue fever virus serotype 4 (DENV-4), Japanese encephalitis virus (JEV), and/or Zika virus (ZIKV).

28. The method of claim 27, wherein the pharmaceutical composition is capable of treating or preventing more than one subtypes of the flaviviruses infection in the subject.


SEQUENCE LISTING (117 sequences)

SEQ ID NO: 1 - protein, 8 aa, synthetic construct
GYTFTSNW

SEQ ID NO: 2 - protein, 8 aa, synthetic construct
IDPSDSYT

SEQ ID NO: 3 - protein, 15 aa, synthetic construct
CARGSNPYYYAMDYW

SEQ ID NO: 4 - protein, 7 aa, synthetic construct
QSVSSNY

SEQ ID NO: 5 - protein, 5 aa, synthetic construct
GVSSS

SEQ ID NO: 6 - protein, 12 aa, synthetic construct
CQHYGGSLSWTF

SEQ ID NO: 7 - protein, 8 aa, synthetic construct
GDTFSNSD

SEQ ID NO: 8 - protein, 8 aa, synthetic construct
IIPAYDTT

SEQ ID NO: 9 - protein, 21 aa, synthetic construct
CATDQRDYFGLGNHFNAQHFW

SEQ ID NO: 10 - protein, 6 aa, synthetic construct
QSISNY

SEQ ID NO: 11 - skipped

SEQ ID NO: 12 - protein, 11 aa, synthetic construct
CQQSYSGGYTF

SEQ ID NO: 13 - protein, 6 aa, synthetic construct
QNINSY

SEQ ID NO: 14 - skipped

SEQ ID NO: 15 - protein, 11 aa, synthetic construct [sequence omitted]

SEQ ID NO: 16 - protein, 6 aa, synthetic construct
QDVSNF

SEQ ID NO: 17 - skipped

SEQ ID NO: 18 - protein, 11 aa, synthetic construct
CQQHSTFPLTF

SEQ ID NO: 19 - protein, 126 aa, synthetic construct
EVQLLESGAEVKKPGSSVKVSCQASGDTFSNSDINWVRQAPGQGLEWMGGIIPAYDTTNYARTFQGRVAITADKSTSTVYMELSRLRSEDTAVYYCATDQRDYFGLGNHFNAQHFWGQGTLVTVSS

SEQ ID NO: 20 - protein, 107 aa, synthetic construct
AIRMTQTPLSLSASVGDRVTITCRASQSISNYLNWYQQRPGKAPNLLIYTASNLHSGVPSRFSGSGSGTDFTLTISSLQPEDFATYYCQQSYSGGYTFGQGPSWEIK

SEQ ID NO: 21 - protein, 120 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 22 - protein, 109 aa, synthetic construct
EIVMMQSPLTLSLSPGERATLSCRASQSVSSNYLAWYQQRPGQAPRLLIYGVSSRATGVPVRFSGSGSGTDFTLTISRLEPEDFAVYYCQHYGGSLSWTFGQGTKVEIK

SEQ ID NO: 23 - protein, 400 aa, Dengue virus
MRCVGIGSRDFVEGLSGATWVDVVLEHGSCVTTMAKDKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTPQETWNREDLLVTFKTAHAKKQEVVVLGSQEGAMHTALTGATEIQTSGTTKIFAGHLKCRLKMNKLTLKGMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKKGSSIGK

SEQ ID NO: 24 - protein, 400 aa, Dengue virus
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGW

SEQ ID NO: 25 - protein, 398 aa, Dengue virus
MRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNDTQGVTVEITPQASTVEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRKGSSIGK

SEQ ID NO: 26 - protein, 400 aa, Dengue virus
MRCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDISNHGVTATITPRSPSVEVKLPDYGELTLDCEPRSGIDFNEMILMKMKKKTWLVHKQWFLDLPLPWAAGADTSEVHWNYKERMVTFKVPHAKRQDVTVLGSQEGAMHSALTGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAEYTNSVTNIELEPPFGDSYIVIGVGDSALTLHWFRKGSSIGK

SEQ ID NO: 27 - protein, 405 aa, Japanese encephalitis virus
FNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGLFGKGSIDTCAKFSCTSKAIGRTIQPENIKYEVGIFVHGTTTSENHGNYSAQVGASQAAKFTVTPNAPSITLKLGDYGEVTLDCEPRSGLNTEAFYVMTVGSKSFLVHREWFHDLALPWTSPSSTAWRNRELLMEFEEAHATKQSVVALGSQEGGLHQALAGAIVVEYSSSVKLTSGHLKCRLKMDKLALKGTTYGMCTEKFSFAKNPADTGHGTVVIELSYSGSDGPCKIPIASVASLNDMTPVGRLVTVNPFVATSSANSKVLVEMEPPFGDSYIVVGRGDKQINHHWHKAGSTLGK

SEQ ID NO: 28 - protein, 409 aa, Zika virus
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGK

SEQ ID NO: 29 - DNA, 59 bp, synthetic construct
  source 1..59: mol_type = other DNA
ccttgtccttgttttaaccggtgtccagtgtcaggtgcagctggtgcagcctggagcag

SEQ ID NO: 30 - DNA, 51 bp, synthetic construct
  source 1..51: mol_type = other DNA
ccgatgggcccttggtcgacgcggagctcactgtcacgctggtgccctggc

SEQ ID NO: 31 - DNA, 58 bp, synthetic construct
  source 1..58: mol_type = other DNA
gccagggcaccagcgtgacagtgagctccgcgtcgaccaagggcccatcggtcttccc

SEQ ID NO: 32 - DNA, 52 bp, synthetic construct
  source 1..52: mol_type = other DNA
caggctgcaccagctgcacctgacactggacaccggttaaaacaaggacaag

SEQ ID NO: 33 - DNA, 42 bp, synthetic construct
  source 1..42: mol_type = other DNA
cttcgggttcagcttgattttccttgtccttgttttaaccgg

SEQ ID NO: 34 - DNA, 65 bp, synthetic construct
  source 1..65: mol_type = other DNA
gatgatgatgatgggacccagttttgtcacaagatttgggctcaactttcttgtccaccttggtg

SEQ ID NO: 35 - DNA, 51 bp, synthetic construct
  source 1..51: mol_type = other DNA
gggtcccatcatcatcatcatcatcatcattgagcggccgctcgagtctag

SEQ ID NO: 36 - DNA, 41 bp, synthetic construct
  source 1..41: mol_type = other DNA
attagaattcacactggacaccggttaaaacaaggacaagg

SEQ ID NO: 37 - DNA, 62 bp, synthetic construct
  source 1..62: mol_type = other DNA
gatgatgatgatgatgatgatgggacccgcagagttcaggtgctgggcacggtgggcatgtg

SEQ ID NO: 38 - DNA, 64 bp, synthetic construct
  source 1..64: mol_type = other DNA
cacacatgcccaccgtgcccagcacctgaactctgcgggtcccatcatcatcatcatcatcatc

SEQ ID NO: 39 - DNA, 52 bp, synthetic construct
  source 1..52: mol_type = other DNA
ggcaccagcgtgacagtgagctccgcgtcgaccaagggcccatcggtcttcc

SEQ ID NO: 40 - DNA, 44 bp, synthetic construct
  source 1..44: mol_type = other DNA
ctagactcgagcggccgctcatttacccggagacagggagaggc

SEQ ID NO: 41 - DNA, 44 bp, synthetic construct
  source 1..44: mol_type = other DNA
gcctctccctgtctccgggtaaatgagcggccgctcgagtctag

SEQ ID NO: 42 - DNA, 51 bp, synthetic construct; identical to SEQ ID NO: 30
  source 1..51: mol_type = other DNA

SEQ ID NO: 43 - DNA, 54 bp, synthetic construct
  source 1..54: mol_type = other DNA
gtccttgttttaaccggtgtccagtgtgaaatagtgatgatgcagtctccactc

SEQ ID NO: 44 - DNA, 55 bp, synthetic construct
  source 1..55: mol_type = other DNA
gaagacagatggcgccgccaccgtacgtttgatctccaccttggtcccttggccg

SEQ ID NO: 45 - DNA, 54 bp, synthetic construct
  source 1..54: mol_type = other DNA
cgttcggccaagggaccaaggtggagatcaaacgtacggtggcggcgccatctg

SEQ ID NO: 46 - DNA, 55 bp, synthetic construct
  source 1..55: mol_type = other DNA
gtggagactgcatcatcactatttcacactggacaccggttaaaacaaggacaag

SEQ ID NO: 47 - DNA, 24 bp, synthetic construct
  source 1..24: mol_type = other DNA
acaggtgcccactcccaggtgcag

SEQ ID NO: 48 - DNA, 23 bp, synthetic construct
  source 1..23: mol_type = other DNA
aaggtgtccagtgtgargtgcag

SEQ ID NO: 49 - DNA, 27 bp, synthetic construct
  source 1..27: mol_type = other DNA
cccagatgggtcctgtcccaggtgcag

SEQ ID NO: 50 - DNA, 24 bp, synthetic construct
  source 1..24: mol_type = other DNA
caaggagtctgttccgaggtgcag

SEQ ID NO: 51 - DNA, 23 bp, synthetic construct
  source 1..23: mol_type = other DNA
ggaaggtgtgcacgccgctggtc

SEQ ID NO: 52 - DNA, 25 bp, synthetic construct
  source 1..25: mol_type = other DNA
atgaggstcccygctcagctgctgg

SEQ ID NO: 53 - DNA, 25 bp, synthetic construct
  source 1..25: mol_type = other DNA
atttctctgttgctctggatctctg

SEQ ID NO: 54 - DNA, 24 bp, synthetic construct
  source 1..24: mol_type = other DNA
gtttctcgtagtctgctttgctca

SEQ ID NO: 55 - DNA, 48 bp, synthetic construct
  source 1..48: mol_type = other DNA
cttgtccttgttttaaccggtgtccagtgtcaggtgcagctggtgcag

SEQ ID NO: 56 - DNA, 48 bp, synthetic construct
  source 1..48: mol_type = other DNA
cttgtccttgttttaaccggtgtccagtgtcaggtgcaggtggtgcag

SEQ ID NO: 57 - DNA, 48 bp, synthetic construct
  source 1..48: mol_type = other DNA
cttgtccttgttttaaccggtgtccagtgtgaggtgcagctggtgcag

SEQ ID NO: 58 - DNA, 48 bp, synthetic construct
  source 1..48: mol_type = other DNA
cttgtccttgttttaaccggtgtccagtgtcaggttcagctggtgcag

SEQ ID NO: 59 - DNA, 48 bp, synthetic construct
  source 1..48: mol_type = other DNA
cttgtccttgttttaaccggtgtccagtgtcaggtccagctggtacag

SEQ ID NO: 60 - DNA, 48 bp, synthetic construct
  source 1..48: mol_type = other DNA
cttgtccttgttttaaccggtgtccagtgtgaggtgcagctggtggag

SEQ ID NO: 61 - DNA, 48 bp, synthetic construct
  source 1..48: mol_type = other DNA
cttgtccttgttttaaccggtgtccagtgtgaggtgcagctgttggag

SEQ ID NO: 62 - DNA, 48 bp, synthetic construct
  source 1..48: mol_type = other DNA
cttgtccttgttttaaccggtgtccagtgtcaggtgcagctggtggag

SEQ ID NO: 63 - DNA, 48 bp, synthetic construct
  source 1..48: mol_type = other DNA
cttgtccttgttttaaccggtgtccagtgtgaagtgcagctggtggag

SEQ ID NO: 64 - DNA, 48 bp, synthetic construct
  source 1..48: mol_type = other DNA
cttgtccttgttttaaccggtgtccagtgtcaggtgcagctgcaggag

SEQ ID NO: 65 - DNA, 50 bp, synthetic construct
  source 1..50: mol_type = other DNA
cttgtccttgttttaaccggtgtccagtgtcaggtgcagctacagcagtg

SEQ ID NO: 66 - DNA, 48 bp, synthetic construct
  source 1..48: mol_type = other DNA
cttgtccttgttttaaccggtgtccagtgtcagctgcagctgcaggag

SEQ ID NO: 67 - DNA, 48 bp, synthetic construct
  source 1..48: mol_type = other DNA
cttgtccttgttttaaccggtgtccagtgtcaggtrcagctgcagcag

SEQ ID NO: 68 - DNA, 41 bp, synthetic construct
  source 1..41: mol_type = other DNA
gaccgatgggcccttggtcgacgckgargagacggtgacca

SEQ ID NO: 69 - DNA, 42 bp, synthetic construct
  source 1..42: mol_type = other DNA
gaccgatgggcccttggtcgacgctgaggagacagagaccag

SEQ ID NO: 70 - DNA, 44 bp, synthetic construct
  source 1..44: mol_type = other DNA
gaccgatgggcccttggtcgacgctgaagagacggtgaccattg

SEQ ID NO: 71 - DNA, 42 bp, synthetic construct
  source 1..42: mol_type = other DNA
gaccgatgggcccttggtcgacgcggagctcactgtcacgct

SEQ ID NO: 72 - DNA, 43 bp, synthetic construct
  source 1..43: mol_type = other DNA
gaccgatgggcccttggtcgacgctgargagacggtgaccgtg

SEQ ID NO: 73 - DNA, 42 bp, synthetic construct
  source 1..42: mol_type = other DNA
gaccgatgggcccttggtcgacgctgaggagacggtgagtgt

SEQ ID NO: 74 - DNA, 50 bp, synthetic construct
  source 1..50: mol_type = other DNA
cttgtccttgttttaaccggtgtccagtgtgacatccagatgacccagtc

SEQ ID NO: 75 - DNA, 51 bp, synthetic construct
  source 1..51: mol_type = other DNA
cttgtccttgttttaaccggtgtccagtgtgacatccagttgacccagtct

SEQ ID NO: 76 - DNA, 42 bp, synthetic construct
  source 1..42: mol_type = other DNA
cttgtccttgttttaaccggtgtccagtgtgycatcyggatg

SEQ ID NO: 77 - DNA, 50 bp, synthetic construct
  source 1..50: mol_type = other DNA
cttgtccttgttttaaccggtgtccagtgtgccatccggatgacccagtc

SEQ ID NO: 78 - DNA, 50 bp, synthetic construct
  source 1..50: mol_type = other DNA
cttgtccttgttttaaccggtgtccagtgtgatattgtgatgacccagac

SEQ ID NO: 79 - DNA, 50 bp, synthetic construct
  source 1..50: mol_type = other DNA
cttgtccttgttttaaccggtgtccagtgtgatattgtgatgactcagtc

SEQ ID NO: 80 - DNA, 50 bp, synthetic construct
  source 1..50: mol_type = other DNA
cttgtccttgttttaaccggtgtccagtgtgatgttgtgatgactcagtc

SEQ ID NO: 81 - DNA, 50 bp, synthetic construct
  source 1..50: mol_type = other DNA
cttgtccttgttttaaccggtgtccagtgtgaratygtgytgacmcagwc

SEQ ID NO: 82 - DNA, 48 bp, synthetic construct
  source 1..48: mol_type = other DNA
cttgtccttgttttaaccggtgtccagtgtgaaatwgtgwtgayscag

SEQ ID NO: 83 - DNA, 50 bp, synthetic construct
  source 1..50: mol_type = other DNA
cttgtccttgttttaaccggtgtccagtgtgaaatagtgatgacgcagtc

SEQ ID NO: 84 - DNA, 48 bp, synthetic construct
  source 1..48: mol_type = other DNA
cttgtccttgttttaaccggtgtccagtgtgaaatagtgatgaygcag

SEQ ID NO: 85 - DNA, 51 bp, synthetic construct
  source 1..51: mol_type = other DNA
cttgtccttgttttaaccggtgtccagtgtgaaattgtgttgacrcagtct

SEQ ID NO: 86 - DNA, 50 bp, synthetic construct
  source 1..50: mol_type = other DNA
cttgtccttgttttaaccggtgtccagtgtgacatcgtgatgacccagtc

SEQ ID NO: 87 - DNA, 38 bp, synthetic construct
  source 1..38: mol_type = other DNA
gatggcgccgccaccgtacgyttgatytcsaccttkgt

SEQ ID NO: 88 - DNA, 38 bp, synthetic construct
  source 1..38: mol_type = other DNA
gatggcgccgccaccgtacgyttkaaytcsaccttkgt

SEQ ID NO: 89 - DNA, 38 bp, synthetic construct
  source 1..38: mol_type = other DNA
gatggcgccgccaccgtacgygtgatytcsaccttkgt

SEQ ID NO: 90 - DNA, 38 bp, synthetic construct
  source 1..38: mol_type = other DNA
gatggcgccgccaccgtacgyttgatytccagcttkgt

SEQ ID NO: 91 - DNA, 38 bp, synthetic construct
  source 1..38: mol_type = other DNA
gatggcgccgccaccgtacgyttgatccccagcttkgt

SEQ ID NO: 92 - DNA, 38 bp, synthetic construct
  source 1..38: mol_type = other DNA
gatggcgccgccaccgtacgngtgatytccagcttkgt

SEQ ID NO: 93 - DNA, 38 bp, synthetic construct
  source 1..38: mol_type = other DNA
gatggcgccgccaccgtacgyttgatctcccagcttgg

SEQ ID NO: 94 - DNA, 38 bp, synthetic construct
  source 1..38: mol_type = other DNA
gatggcgccgccaccgtacgnktgatytcnacgcttgg

SEQ ID NO: 95 - DNA, 39 bp, synthetic construct
  source 1..39: mol_type = other DNA
gatggcgccgccaccgtacgtttgatatccactttggtc

SEQ ID NO: 96 - DNA, 38 bp, synthetic construct
  source 1..38: mol_type = other DNA
gatggcgccgccaccgtacgyctgatytcnaccttkgt

SEQ ID NO: 97 - DNA, 39 bp, synthetic construct
  source 1..39: mol_type = other DNA
gatggcgccgccaccgtacgyttaatctccagtcgtgtc

SEQ ID NO: 98 - DNA, 29 bp, synthetic construct
  source 1..29: mol_type = other DNA
gcywscaccaagggcccatcggtcttccc

SEQ ID NO: 99 - DNA, 33 bp, synthetic construct
  source 1..33: mol_type = other DNA
tcatttacccggagacagggagaggctcttctg

SEQ ID NO: 100 - DNA, 35 bp, synthetic construct
  source 1..35: mol_type = other DNA
tgaactcctgggaggaccgtcagtcttcctcttcc

SEQ ID NO: 101 - DNA, 34 bp, synthetic construct
  source 1..34: mol_type = other DNA
ggaagactgacggtcctcccaggagttcaggtgc

SEQ ID NO: 102 - DNA, 33 bp, synthetic construct
  source 1..33: mol_type = other DNA
atggccagcgacagcgcatgccccacacagggc

SEQ ID NO: 103 - DNA, 33 bp, synthetic construct
  source 1..33: mol_type = other DNA
gccctgtgtggggcatgcgctgtcgctggccat

SEQ ID NO: 104 - DNA, 32 bp, synthetic construct
  source 1..32: mol_type = other DNA
acagcagatgccccgcacagggcgaggcttac

SEQ ID NO: 105 - DNA, 32 bp, synthetic construct
  source 1..32: mol_type = other DNA
gtaagcctcgccctgtgcggggcatctgctgt

SEQ ID NO: 106 - DNA, 33 bp, synthetic construct
  source 1..33: mol_type = other DNA
ctggacaagcagtccgccacccagtacgtgtgc

SEQ ID NO: 107 - DNA, 33 bp, synthetic construct
  source 1..33: mol_type = other DNA
gcacacgtactgggtggcggactgcttgtccag

SEQ ID NO: 108 - DNA, 31 bp, synthetic construct
  source 1..31: mol_type = other DNA
ggatagaggctggggcgctggctgcggcctg

SEQ ID NO: 109 - DNA, 31 bp, synthetic construct
  source 1..31: mol_type = other DNA
caggccgcagccagcgccccagcctctatcc

SEQ ID NO: 110 - DNA, 33 bp, synthetic construct
  source 1..33: mol_type = other DNA
ctggactgcgagcctgcaaccggcctggatttc

SEQ ID NO: 111 - DNA, 33 bp, synthetic construct
  source 1..33: mol_type = other DNA
gaaatccaggccggttgcaggctcgcagtccag

SEQ ID NO: 112 - DNA, 36 bp, synthetic construct
  source 1..36: mol_type = other DNA
tggcatgccggcgctgatgcaggcacaccccactgg

SEQ ID NO: 113 - DNA, 37 bp, synthetic construct
  source 1..37: mol_type = other DNA
ccagtggggtgtgcctgcatcagcgccggcatgccag

SEQ ID NO: 114 - DNA, 34 bp, synthetic construct
  source 1..34: mol_type = other DNA
caagctgcggctgaaggccgtgtcctacagcctg

SEQ ID NO: 115 - DNA, 34 bp, synthetic construct
  source 1..34: mol_type = other DNA
caggctgtaggacacggccttcagccgcagcttg

SEQ ID NO: 116 - DNA, 35 bp, synthetic construct
  source 1..35: mol_type = other DNA
ccgccaaccctgtgatcgccgagagcaccgagaac

SEQ ID NO: 117 - DNA, 35 bp, synthetic construct
  source 1..35: mol_type = other DNA
gttctcggtgctctcggcgatcacagggttggcgg